SEQUENCE LISTING (78 sequences)

SEQ ID NO: 1 - DNA, 882 bp, Lawsonia intracellularis
  source 1..882: mol_type = unassigned DNA
atgtctcttgtcattaataacaacctgatggccgtcaatgctcaacgtaacttaagcaagtcttatggagaactgagttcttctgttcgaaaactttcttcaggtcttcgtgtaggaactgctgctgatgactcagcagggttagccattcgagaactcatgagatctgacattgcaacaacacaacaaggaatacgaaatgcgaatgatgctatttctatgattcaaactgcggatggtgcacttggagtcatcgatgaaaagctcattcgaatgaaagaacttgctgaacaagctgctacaggtacatataactccactcagcgtatgattattgactctgaatatcaagctatggcctcagaaattactcgtattgctaatgcgacagaatttaatggtataaaacttcttgatggttcattatcaggtaatcatgatgggaaaaaaataaattcaactggtgcagtacgtatccactttgggacatctaacagctctgctgaagattactatgatattaaaattggtggctctacagcttctgcattaggacttggtaatacagtaaaaggtgcgggtgctacagtctctactcaagctgcagcacaaaatgccttaaaagctatagataatgccattgtttcaaaagataaaattcgagcacaccttggtggattacaaaatagacttgaagctacagttgataatttaagtatacaaaatgaaaacttacaagctgctgaatctcgtatatctgatatagatgtaagccaagaaatgacacaatttgtacgtaaccaaatacttacacaaacaggtgttgctatgctttcacaagctaattctctaccacgtatggctcagcaacttattggctaa

SEQ ID NO: 2 - protein, 293 aa, Lawsonia intracellularis
MSLVINNNLMAVNAQRNLSKSYGELSSSVRKLSSGLRVGTAADDSAGLAIRELMRSDIATTQQGIRNANDAISMIQTADGALGVIDEKLIRMKELAEQAATGTYNSTQRMIIDSEYQAMASEITRIANATEFNGIKLLDGSLSGNHDGKKINSTGAVRIHFGTSNSSAEDYYDIKIGGSTASALGLGNTVKGAGATVSTQAAAQNALKAIDNAIVSKDKIRAHLGGLQNRLEATVDNLSIQNENLQAAESRISDIDVSQEMTQFVRNQILTQTGVAMLSQANSLPRMAQQLIG

SEQ ID NO: 3 - protein, 301 aa, synthetic construct
  REGION 1..301: note = Cloned peptide
MHHHHHHGSSLVINNNLMAVNAQRNLSKSYGELSSSVRKLSSGLRVGTAADDSAGLAIRELMRSDIATTQQGIRNANDAISMIQTADGALGVIDEKLIRMKELAEQAATGTYNSTQRMIIDSEYQAMASEITRIANATEFNGIKLLDGSLSGNHDGKKINSTGAVRIHFGTSNSSAEDYYDIKIGGSTASALGLGNTVKGAGATVSTQAAAQNALKAIDNAIVSKDKIRAHLGGLQNRLEATVDNLSIQNENLQAAESRISDIDVSQEMTQFVRNQILTQTGVAMLSQANSLPRMAQQLIG

SEQ ID NO: 4 - DNA, 2556 bp, Lawsonia intracellularis
  source 1..2556: mol_type = unassigned DNA
atggcatacctatctatttcaaaaaatcaatgtaagtcttttttaataactctagtaactatatttataatgacatcaataccacaactagctgaggctgttgaacactttgctaatggtgtccccacagtagtacaagatgttaatgtcccagctgactcatactttggtggtgctgactcagctgtaggtccaaacccaatagcttctactcaccttacaatttctacaactcaagggtttggacaaaatgcactagaatttgttgtgggtggtagccttgcaaatggtaatggaaatccagccaacataaatggagatatagtccttattgttgaaaatacaaatactcagaatagtattattggtggtagtatggcaaatgctgcccctgtaactattggtggctctatttttatgactcttagaaatgttactgcagtagatccaatttttggtggctctgtagatgtacgctttttcgcccaacagcaacctaatgaggatcaacttgtaggtggagatattaatataaatctagagaatgtaacaactccagagttttatggtctaggctacgcaaatggtgtaatacctgttaacgtcctcaatagaaattttttggtagctgttcagggaaatataactacaaatatttctaattctaacattgcaaccgttatgttaggttcacactatgatacaactatggctgtaggaggtaatggaacaataaatgttgataattcaacaattggttatctttcagctagcaatagtagcgactttgttaaccctgacttaacaaacactgttacttttaatattggtcccaacaacagaatagcaaatatttttgcaagcaataatggtgttatcccacattttattgtaaatatggatggatctggaacagaaatacaagagttaacacttggtaatgtaattcgaggtggccttgtacttacttcagagctaaatctatctcaaggaacaataaataatttaatcactggtaatgaatattacgatagatcaggattaagaactactgtaaatgttagaggaggtacaattggcgtattgacttcaggaggttctgactattcagaattaaacttcattcctggagaaatatccactatattagcaacaaattcaataggtaaccaagattttgcatctctttctcaagttacaatacaccaaggtgctgaaactctttggggaatgagagatgaagtatttgaacttcaaactaacaatctacagttaggtggtgaactatttattcctgctgatggaactggaggagtagccctaatcacaaatcatattattgcaaattcaggtgtgataactccggtaaatatgtctccagaaaggatgacccctatcattggatttttagaacctactggtgaggtagcacagttaacaatatatggaccacttacagttaaccttagccattctccagaaattcttgggaaaattattacacaacctatccctattgcagttactaatagtgatgtttttggtacttctaaactatttgtggaacataacacaaaaggactaatttggagtgatatcatttttaatcctcaagataaaacatggtatctaactaactttagaggttctgaagacttctacggactttcagcagcacgggaagcatctaattggttaagacaacaacatatctggagcctacaacgtcgctctaataaattattagatcatggtgtagatgggttatggatgaatgttcaaggtggttatgaaaagcttgatgcagcaattggtgatgctaagatgccttggattatggcaagcttaggatatgattttatgcataagttaagtgatttttataacttaaaagcactttatggattcggatttggatttgctacaggtaaaaataaatggaataccataaactcaactactaatgatatctacatggggctggttggtgcctatgttggccttatgcatgaagccacaggcctttatggtacagtatccggacagtttgcaactaaccgtacaaaaacaaaatgtacaggctttgatgaaacctataactggaaagaaaatgtaccaacagaagctattgaaattggttggaaatggagcattgatgagtttaaaataaacccacgtggacaagttatttttgaacaattatctaaacatcactttagtctctcacaagaaggtgatactgctatcttagataaagaattcttaactacaaccgttataggtatctctggagaatatgatttagatttaagaagtaaaataataaagcttcaagctagtgttgactggattaaaggcatctctggtgactttgcagctaaatccgaagttcttaatatgaagtttaaagataaaaatgatactagtacatttagaggaacactgggtgctagtgcacaacttctagaaaactttgaagttcaccttgatatttttggtgatcttggcaatgataaaggtattggtggacaggtaggagctacttatagattctaa

SEQ ID NO: 5 - protein, 851 aa, Lawsonia intracellularis
MAYLSISKNQCKSFLITLVTIFIMTSIPQLAEAVEHFANGVPTVVQDVNVPADSYFGGADSAVGPNPIASTHLTISTTQGFGQNALEFVVGGSLANGNGNPANINGDIVLIVENTNTQNSIIGGSMANAAPVTIGGSIFMTLRNVTAVDPIFGGSVDVRFFAQQQPNEDQLVGGDININLENVTTPEFYGLGYANGVIPVNVLNRNFLVAVQGNITTNISNSNIATVMLGSHYDTTMAVGGNGTINVDNSTIGYLSASNSSDFVNPDLTNTVTFNIGPNNRIANIFASNNGVIPHFIVNMDGSGTEIQELTLGNVIRGGLVLTSELNLSQGTINNLITGNEYYDRSGLRTTVNVRGGTIGVLTSGGSDYSELNFIPGEISTILATNSIGNQDFASLSQVTIHQGAETLWGMRDEVFELQTNNLQLGGELFIPADGTGGVALITNHIIANSGVITPVNMSPERMTPIIGFLEPTGEVAQLTIYGPLTVNLSHSPEILGKIITQPIPIAVTNSDVFGTSKLFVEHNTKGLIWSDIIFNPQDKTWYLTNFRGSEDFYGLSAAREASNWLRQQHIWSLQRRSNKLLDHGVDGLWMNVQGGYEKLDAAIGDAKMPWIMASLGYDFMHKLSDFYNLKALYGFGFGFATGKNKWNTINSTTNDIYMGLVGAYVGLMHEATGLYGTVSGQFATNRTKTKCTGFDETYNWKENVPTEAIEIGWKWSIDEFKINPRGQVIFEQLSKHHFSLSQEGDTAILDKEFLTTTVIGISGEYDLDLRSKIIKLQASVDWIKGISGDFAAKSEVLNMKFKDKNDTSTFRGTLGASAQLLENFEVHLDIFGDLGNDKGIGGQVGATYRF

SEQ ID NO: 6 - protein, 866 aa, synthetic construct
  REGION 1..866: note = Cloned peptide
MHHHHHHSSGLVPRGSGMKETAAAKFERQHMDSPDLGTDDDDKAMAEAVEHFANGVPTVVQDVNVPADSYFGGADSAVGPNPIASTHLTISTTQGFGQNALEFVVGGSLANGNGNPANINGDIVLIVENTNTQNSIIGGSMANAAPVTIGGSIFMTLRNVTAVDPIFGGSVDVRFFAQQQPNEDQLVGGDININLENVTTPEFYGLGYANGVIPVNVLNRNFLVAVQGNITTNISNSNIATVMLGSHYDTTMAVGGNGTINVDNSTIGYLSASNSSDFVNPDLTNTVTFNIGPNNRIANIFASNNGVIPHFIVNMDGSGTEIQELTLGNVIRGGLVLTSELNLSQGTINNLITGNEYYDRSGLRTTVNVRGGTIGVLTSGGSDYSELNFIPGEISTILATNSIGNQDFASLSQVTIHQGAETLWGMRDEVFELQTNNLQLGGELFIPADGTGGVALITNHIIANSGVITPVNMSPERMTPIIGFLEPTGEVAQLTIYGPLTVNLSHSPEILGKIITQPIPIAVTNSDVFGTSKLFVEHNTKGLIWSDIIFNPQDKTWYLTNFRGSEDFYGLSAAREASNWLRQQHIWSLQRRSNKLLDHGVDGLWMNVQGGYEKLDAAIGDAKMPWIMASLGYDFMHKLSDFYNLKALYGFGFGFATGKNKWNTINSTTNDIYMGLVGAYVGLMHEATGLYGTVSGQFATNRTKTKCTGFDETYNWKENVPTEAIEIGWKWSIDEFKINPRGQVIFEQLSKHHFSLSQEGDTAILDKEFLTTTVIGISGEYDLDLRSKIIKLQASVDWIKGISGDFAAKSEVLNMKFKDKNDTSTFRGTLGASAQLLENFEVHLDIFGDLGNDKGIGGQVGATYRF

SEQ ID NO: 7 - DNA, 1658 bp, Lawsonia intracellularis
  source 1..1658: mol_type = unassigned DNA
atggataaaattatacattataaaactaaaacattagtacattcaattttttttcttctttttttattgacctctctttatctaacaatacattttgtttatgcacaaaaactaacagagcaaaacagtgatgaggaccttagtacagaacaaccagtacatggtggacgtattcgtttaggaacaatagcagaaccaataaatttaattccttacttgtctacagacggtacttcacacgaaattgctgacttactatttgtatctgcacttgagtatgataaagatttacaagtagtaccactagcagcaaagtcatatgaagttttgaatgatggaaaattattacgatttattatgcgtgaagatgtgttttggcaagatggagtgcaacttactgttgacgatatagaatttacatataaattaatgttagagcctaacacacctacagcttatgcagaggactttctcactattaaggagtttaaaaaaacaggccgttttacttttgaagtgtattatgaaaagccatatgctagagctctaatgacatggatgggctcaattttaccaaaacacattcttgaggggcaagatattaccaaaacaccttttgctagaaatcctataggtgctggaccatataaattaaaaagttgggaaacaggttctcgccttattcttgaggcatctgatagttactttaaaggtaagccatatatctcagaagttatttatacagttatcccagatagttcaacaatgttccttgaattaagagcagggaatttagatatgatgggccttacaccacaacaatacttaaagcaaactaaaggtccacagtgggaaaaaaattggaagaaatatagatatcttgctttttcatatgcttacttaggctttaatttaaacaagcctatgtttcaagatatattaactcgtcaaggaatttcccatgctattgatcgtcaggctattgtagataatgttcttcttggtgaaggagtcgtttcctttggtccttacaagccaggaacatgggtatacaacacacaccttaaaccaatagaatataatccagaaaaagctcgacaattatttacacaggcaggatggaaagatacaggcaatggtgttcttcaaagagatggtaggccttttacatttactatacttgtgaatcaagggaatgaacaaagagcacgagtagcaagtattattcaaagtcagttaaaagaagtaggaattgaagtacaaattaggactgtagagtgggctgcttttcttaaagagtttatagataaaggaagatatgatgctgttgttcttggatggtctattacacaagatcctgatatttatgatgtatggcattcttcaaaagcacatgaaggaggattaaacttcatggggtataaaaatacggaacttgatgcactccttgtagaagcaagaacagaacttgatcaagctaaacgcaagccattatatgataaaatacaagaaatacttcatcatgatcaaccatactgttttctttttgtaccttattctttacctattataaaatctaaatttcatgggataaaaccagcaccagcaggtattatgtataatcttgatcagtggtggattcctaaaaagcttcaata

SEQ ID NO: 8 - protein, 552 aa, Lawsonia intracellularis
MDKIIHYKTKTLVHSIFFLLFLLTSLYLTIHFVYAQKLTEQNSDEDLSTEQPVHGGRIRLGTIAEPINLIPYLSTDGTSHEIADLLFVSALEYDKDLQVVPLAAKSYEVLNDGKLLRFIMREDVFWQDGVQLTVDDIEFTYKLMLEPNTPTAYAEDFLTIKEFKKTGRFTFEVYYEKPYARALMTWMGSILPKHILEGQDITKTPFARNPIGAGPYKLKSWETGSRLILEASDSYFKGKPYISEVIYTVIPDSSTMFLELRAGNLDMMGLTPQQYLKQTKGPQWEKNWKKYRYLAFSYAYLGFNLNKPMFQDILTRQGISHAIDRQAIVDNVLLGEGVVSFGPYKPGTWVYNTHLKPIEYNPEKARQLFTQAGWKDTGNGVLQRDGRPFTFTILVNQGNEQRARVASIIQSQLKEVGIEVQIRTVEWAAFLKEFIDKGRYDAVVLGWSITQDPDIYDVWHSSKAHEGGLNFMGYKNTELDALLVEARTELDQAKRKPLYDKIQEILHHDQPYCFLFVPYSLPIIKSKFHGIKPAPAGIMYNLDQWWIPKKLQ

SEQ ID NO: 9 - protein, 565 aa, synthetic construct
  REGION 1..565: note = Cloned peptide
MHHHHHHSSGLVPRGSGMKETAAAKFERQHMDSPDLGTDDDDKAMDSDEDLSTEQPVHGGRIRLGTIAEPINLIPYLSTDGTSHEIADLLFVSALEYDKDLQVVPLAAKSYEVLNDGKLLRFIMREDVFWQDGVQLTVDDIEFTYKLMLEPNTPTAYAEDFLTIKEFKKTGRFTFEVYYEKPYARALMTWMGSILPKHILEGQDITKTPFARNPIGAGPYKLKSWETGSRLILEASDSYFKGKPYISEVIYTVIPDSSTMFLELRAGNLDMMGLTPQQYLKQTKGPQWEKNWKKYRYLAFSYAYLGFNLNKPMFQDILTRQGISHAIDRQAIVDNVLLGEGVVSFGPYKPGTWVYNTHLKPIEYNPEKARQLFTQAGWKDTGNGVLQRDGRPFTFTILVNQGNEQRARVASIIQSQLKEVGIEVQIRTVEWAAFLKEFIDKGRYDAVVLGWSITQDPDIYDVWHSSKAHEGGLNFMGYKNTELDALLVEARTELDQAKRKPLYDKIQEILHHDQPYCFLFVPYSLPIIKSKFHGIKPAPAGIMYNLDQWWIPTRAPPPPPLRSGC

SEQ ID NO: 10 - DNA, 1197 bp, Lawsonia intracellularis
  source 1..1197: mol_type = unassigned DNA
gagatagttatggctaatgttagtggaatccctgcaccacgattactttccacaacaaatcaaatgaccaatgcagctgctggtaatactaatagagctaccggtagtatgaacggtcgtaatctcacacaaataaaaacacctcagtccatgattgataatgcttcagaagaattaacaacttctcttgaatctaaaagcagtgacgactttgcaattaaagatcgtaaaagacaagggaaaggatctgattctctattaaaaatggttcaagaatatacagagctgacgaatgatgatacccgtaatgctaaaagagctatgttatcccaggtattacgtgcaagtcaaagttcacaagatgtactcgaaaaaacattagaacaattttctaataaaacagatgcttgggcttctcttgcagaaattgcacaagaatatggtgcagaatctccacagccaacaggattaaaatctgtattagatgctatggagacattagaaaatgagtttggtgatgaaattaaagcaggactaaaaggagctctaaattcaaaagaatttactgatataggcagtgcagcacagttaagagatctttatacaacaacagtaactataacagctgcacctgatgcagtgttagcaagacttcttgaagaatatgagagtgatgatgatctggatagagccattgatttccttctatctacacttggtggagagcttgaatcagctgatccaagtatggataaagtacatcttcaaagtgtaatgggtgatattgaaaaaacacaacaacttcatagctctcataaacaatgtactacagcccttagcaggtggaaagagaaacataaaggtgggggggaaaatagtacactaactcctttagaaatgatgcgtgaactaattgcactaaaaaatgaaaattttatttctccttcctctatagataaaattgttgatcaagctgatccccaagatattgaaaaagaagtcctttttttacaagagatgttagctgctgtaagaaaatttcccattatggtatttgataatgtcgaaaatcgtgtaagagttatgggtgctgtacaagatgctgttgacgatgctgtaagaagagaagatgaattcctctttcaaaaagaacatcctgatgtaccactacaaccagatgaaaataatatacaataa

SEQ ID NO: 11 - protein, 398 aa, Lawsonia intracellularis
EIVMANVSGIPAPRLLSTTNQMTNAAAGNTNRATGSMNGRNLTQIKTPQSMIDNASEELTTSLESKSSDDFAIKDRKRQGKGSDSLLKMVQEYTELTNDDTRNAKRAMLSQVLRASQSSQDVLEKTLEQFSNKTDAWASLAEIAQEYGAESPQPTGLKSVLDAMETLENEFGDEIKAGLKGALNSKEFTDIGSAAQLRDLYTTTVTITAAPDAVLARLLEEYESDDDLDRAIDFLLSTLGGELESADPSMDKVHLQSVMGDIEKTQQLHSSHKQCTTALSRWKEKHKGGGENSTLTPLEMMRELIALKNENFISPSSIDKIVDQADPQDIEKEVLFLQEMLAAVRKFPIMVFDNVENRVRVMGAVQDAVDDAVRREDEFLFQKEHPDVPLQPDENNIQ

SEQ ID NO: 12 - protein, 403 aa, synthetic construct
  REGION 1..403: note = Cloned peptide
MHHHHHHGSANVSGIPAPRLLSTTNQMTNAAAGNTNRATGSMNGRNLTQIKTPQSMIDNASEELTTSLESKSSDDFAIKDRKRQGKGSDSLLKMVQEYTELTNDDTRNAKRAMLSQVLRASQSSQDVLEKTLEQFSNKTDAWASLAEIAQEYGAESPQPTGLKSVLDAMETLENEFGDEIKAGLKGALNSKEFTDIGSAAQLRDLYTTTVTITAAPDAVLARLLEEYESDDDLDRAIDFLLSTLGGELESADPSMDKVHLQSVMGDIEKTQQLHSSHKQCTTALSRWKEKHKGGGENSTLTPLEMMRELIALKNENFISPSSIDKIVDQADPQDIEKEVLFLQEMLAAVRKFPIMVFDNVENRVRVMGAVQDAVDDAVRREDEFLFQKEHPDVPLQPDENNIQ

SEQ ID NO: 13 - DNA, 1152 bp, Lawsonia intracellularis
  source 1..1152: mol_type = unassigned DNA
atgttgttatatataaataaagaacacattattgatggtcttcaaaaagttgcaaatattattcctgcacgctcaggtgctgcatatttacgtacactttggatgaaagcagaacattcaacgctgacaataatggctacagatgcaaatattgaatttatagggaattatgaagctgatataaaagaaactggacttgtaggagtaaatggaagaaatttaattgatcttattcgtagattacctgcaaaagaactttgtctacgttttgattcaacatcaggaacacttattcttgaacaagaacgtcgtacatataaaatgcctattaatgaccctatttggtttcaacctttaactttattcccttcagaagaaagtgtagtatggtcagctgacttttttcaagaagtcattgatcgagtatcattttgtattagtgatgatgaaggttctgatgctatttcatgtctttatattcaaccttctaatgataactatataaatgtatgtggtttaaatggacatcaatttgctttaacacggtttataaataatgagttaagtgaaaaaattcctagtgaaggaatacttattcctaaaaaatatataacagaacttcgtaaatggttaggagaaacagaaatagaaatagctataacagaaaaacgattttttgcacgaacaatagatagtaaagaaacaataagtgtcccaagggcgatatttacctatcctgattatacagcttttctcactcgactaacaggagacaatatgtcttttcttaaacttcaaagaaaagattgtcttgagtcacttgatcgaatatctatttttaatactgaaggtgatagatgtacctattttgatataaaggaaaatgaactcattctatctgcacaagggcaagacacaggatctgccaatgaatacctagatgttacttatacaggtaatataaataaaatagcatttccgactaaaagtcttatggaaatatttaatcactttgaatcacctactatcactttagcaatgtcaagtgttgaagggccatgtggaataacaggtgatgaagataaagattatactgtaattattatgcctatgaaaattgttgaacaaaattattatacagaagaagtataa

SEQ ID NO: 14 - protein, 383 aa, Lawsonia intracellularis
MLLYINKEHIIDGLQKVANIIPARSGAAYLRTLWMKAEHSTLTIMATDANIEFIGNYEADIKETGLVGVNGRNLIDLIRRLPAKELCLRFDSTSGTLILEQERRTYKMPINDPIWFQPLTLFPSEESVVWSADFFQEVIDRVSFCISDDEGSDAISCLYIQPSNDNYINVCGLNGHQFALTRFINNELSEKIPSEGILIPKKYITELRKWLGETEIEIAITEKRFFARTIDSKETISVPRAIFTYPDYTAFLTRLTGDNMSFLKLQRKDCLESLDRISIFNTEGDRCTYFDIKENELILSAQGQDTGSANEYLDVTYTGNINKIAFPTKSLMEIFNHFESPTITLAMSSVEGPCGITGDEDKDYTVIIMPMKIVEQNYYTEEV

SEQ ID NO: 15 - protein, 392 aa, synthetic construct
  REGION 1..392: note = Cloned peptide
MHHHHHHGSMLLYINKEHIIDGLQKVANIIPARSGAAYLRTLWMKAEHSTLTIMATDANIEFIGNYEADIKETGLVGVNGRNLIDLIRRLPAKELCLRFDSTSGTLILEQERRTYKMPINDPIWFQPLTLFPSEESVVWSADFFQEVIDRVSFCISDDEGSDAISCLYIQPSNDNYINVCGLNGHQFALTRFINNELSEKIPSEGILIPKKYITELRKWLGETEIEIAITEKRFFARTIDSKETISVPRAIFTYPDYTAFLTRLTGDNMSFLKLQRKDCLESLDRISIFNTEGDRCTYFDIKENELILSAQGQDTGSANEYLDVTYTGNINKIAFPTKSLMEIFNHFESPTITLAMSSVEGPCGITGDEDKDYTVIIMPMKIVEQNYYTEEV

SEQ ID NO: 16 - DNA, 1689 bp, Lawsonia intracellularis
  source 1..1689: mol_type = unassigned DNA
atgttcaaaaaaatatatgttttttatatcacacttcttcttatatttttaacatatgttacaccttatgatgtatggagctttgatcttactattcttcatacaaatgatatacattctcaccttggaggaattaaaaaagaatcaggtaatccttgcttcacatctactacaccagattgcgtaggtggaatggcacgtttagcacaatctattttagacattcgtcaatcaacaccaaatactattcttcttgatgcaggagatcaatttgtaggaacagcttttcattctgactttataaatacaccagatcaactcccatttgtaaagtttttaaatagacttggatatgtagctatgtcaccaggaaatcatgagtttgatcatggatgttatgaattttttagtgctataagacaacttaactttcctgttgtagttgcaaacctcacctttacagatcctgaaatgcaatcatcaattactccatggaccattgtagaacgtgaaggaaaacgtattggtatcataggacttattacagaagcaacagccactggatcaagagcatgctctcaagccatttttactaatgctgaacaagcattacgtaatgcaattcaagaaataaaaaaacaaaatgtatttacaattattgtgttaagccatcttggaataaatgtagatatggaacttgctagtaaagtcgatgatgtctctgtttttgtaggaggtcatacacatactttgctttctaacacctaccctaatgcttatgggccttatcctatagtaaaacattcaccttctggacatcctgtacttattgtaacagcaaaagaaaaactagaatatcttggaaggattaatataacatttgatgaacaaggtatccctcaaaaatggaatggagatgttatacgtcttgataagccaattagtaatgatcctgctatagtatccatagcagaactgcttgatagctatggtatacctattaaagaaaagcttgaagtaaaagttggagaaatagctcatcctagaaatccaaattttgatacatcccaaccttcaaatgaacaactagacgaaaaacctttcttttactgtagaaaacaagagggattaacagctaatattattcttgatgcaatattagaagcaggccgctcaaatggagcacaaatagcaattgtaaccactggattaataagaggaaatttacctatagggcttgtacaaaaacttgatgttgttacagctatacctattgaagataaactttatgtaggagatgttacaggaaaaataattcaggaagctatagaaaatggagtctcaaaagtacattgctttgcctttgcaggaacttttcttcaagttgcaggactcagattcactttaaatgctgaaaaacctgtaggtgaacgcattcaatctattgaatatttaaataatgggaaatatgagccactggaccctaataaaacatatcgtgttataattaatggatatcctactgagggacatgatggatttataatgttaaaagacataaaatggacagatatccaaaaaagtccagtagaagctgttataagctatctaaaagaacattctcctctcaatgtaaaaaaagatggacgtattgttaatgtaacacctattattgtccctaatgaataa

SEQ ID NO: 17 - protein, 562 aa, Lawsonia intracellularis
MFKKIYVFYITLLLIFLTYVTPYDVWSFDLTILHTNDIHSHLGGIKKESGNPCFTSTTPDCVGGMARLAQSILDIRQSTPNTILLDAGDQFVGTAFHSDFINTPDQLPFVKFLNRLGYVAMSPGNHEFDHGCYEFFSAIRQLNFPVVVANLTFTDPEMQSSITPWTIVEREGKRIGIIGLITEATATGSRACSQAIFTNAEQALRNAIQEIKKQNVFTIIVLSHLGINVDMELASKVDDVSVFVGGHTHTLLSNTYPNAYGPYPIVKHSPSGHPVLIVTAKEKLEYLGRINITFDEQGIPQKWNGDVIRLDKPISNDPAIVSIAELLDSYGIPIKEKLEVKVGEIAHPRNPNFDTSQPSNEQLDEKPFFYCRKQEGLTANIILDAILEAGRSNGAQIAIVTTGLIRGNLPIGLVQKLDVVTAIPIEDKLYVGDVTGKIIQEAIENGVSKVHCFAFAGTFLQVAGLRFTLNAEKPVGERIQSIEYLNNGKYEPLDPNKTYRVIINGYPTEGHDGFIMLKDIKWTDIQKSPVEAVISYLKEHSPLNVKKDGRIVNVTPIIVPNE

SEQ ID NO: 18 - protein, 571 aa, synthetic construct
  REGION 1..571: note = Cloned peptide
MHHHHHHGSMFKKIYVFYITLLLIFLTYVTPYDVWSFDLTILHTNDIHSHLGGIKKESGNPCFTSTTPDCVGGMARLAQSILDIRQSTPNTILLDAGDQFVGTAFHSDFINTPDQLPFVKFLNRLGYVAMSPGNHEFDHGCYEFFSAIRQLNFPVVVANLTFTDPEMQSSITPWTIVEREGKRIGIIGLITEATATGSRACSQAIFTNAEQALRNAIQEIKKQNVFTIIVLSHLGINVDMELASKVDDVSVFVGGHTHTLLSNTYPNAYGPYPIVKHSPSGHPVLIVTAKEKLEYLGRINITFDEQGIPQKWNGDVIRLDKPISNDPAIVSIAELLDSYGIPIKEKLEVKVGEIAHPRNPNFDTSQPSNEQLDEKPFFYCRKQEGLTANIILDAILEAGRSNGAQIAIVTTGLIRGNLPIGLVQKLDVVTAIPIEDKLYVGDVTGKIIQEAIENGVSKVHCFAFAGTFLQVAGLRFTLNAEKPVGERIQSIEYLNNGKYEPLDPNKTYRVIINGYPTEGHDGFIMLKDIKWTDIQKSPVEAVISYLKEHSPLNVKKDGRIVNVTPIIVPNE

SEQ ID NO: 19 - DNA, 1458 bp, Lawsonia intracellularis
  source 1..1458: mol_type = unassigned DNA
atgcatctatataatactatggaaaagaaaaaagagccacttatacctataatttcaggaaagttaggtatatatgtttgtggaattacagcgtatgatttttctcatattggacatgcacgatctgccatagtttttgatatcttggtgagattattacgatatcaaggatatgatgtaacatttatacgtaattttacagatattgatgataaaattattaatcgtgctaataaagagggccgtagtagtaaagaggttgcagaagaatttataaatgcttttcatgaagatatggatcgccttggtgtgctaaatgcagatattgagcctaaggctacagattatatccctgaaatgatagagtgttgtcaaaaattactagaggctgataaagcatatattacagcttcaggtgacgtttattttagagttcgttctttccctgattatggaaaactttctggtagaactcctgatgagttacgtataggagtacgtattgtacctagtgaggagaaagaagatcctttagattttgttctttggaaagcagctaagcctggagagccttcttgggaaagtccttggggaagagggcgtcctgggtggcatattgaatgttcagcaatgagtgaaaagtgttggccacttcctcttgatatacatgggggaggaattgatcttatttttccacaccatgaaaatgagattgctcagactgaatcaattgtaaataagccgttggctaaaatttggatgcataatgggcttgttcaagtaaattcagaaaaaatgtcaaaatctcttggaaattttaagattgttagagatatattagaagcatatctcccagaaacattacgttttttccttttaaaaaagcattatagaagccctatagatttttcttttgaaggaatgaatgagacagaacgaagtcaaaaaagagtgtatgaatgtattgctgaggtagataaagcacttgaaagaaaatcttgggactcaggtggttcatctagctctattttagctgagttagatgaacaattttcactatttatgtctgcgctagaagatgattgcaatacagctgctggattaggtcatttatttaatattatccatatagttagacgtgctcttgatgataaagctctttactccacaactgatgggaaggtggtttttgaacaatttcgtgagattatacgaaaagtagatattttattaggtgtatttggtcaaaaacctaatagttttttacaagatctcaaaacaattcgcattattagaaataaaattgatgttaatcaagtagaagagttacttagtaagagaaggcaagcaagagaggaaaaaaattttgttcaggcagatgaagttcgtaataccttagcttcattaggtatagaaatacgtgacacttcagaaggtcaggtgtgggatattttataa

SEQ ID NO: 20 - protein, 485 aa, Lawsonia intracellularis
MHLYNTMEKKKEPLIPIISGKLGIYVCGITAYDFSHIGHARSAIVFDILVRLLRYQGYDVTFIRNFTDIDDKIINRANKEGRSSKEVAEEFINAFHEDMDRLGVLNADIEPKATDYIPEMIECCQKLLEADKAYITASGDVYFRVRSFPDYGKLSGRTPDELRIGVRIVPSEEKEDPLDFVLWKAAKPGEPSWESPWGRGRPGWHIECSAMSEKCWPLPLDIHGGGIDLIFPHHENEIAQTESIVNKPLAKIWMHNGLVQVNSEKMSKSLGNFKIVRDILEAYLPETLRFFLLKKHYRSPIDFSFEGMNETERSQKRVYECIAEVDKALERKSWDSGGSSSSILAELDEQFSLFMSALEDDCNTAAGLGHLFNIIHIVRRALDDKALYSTTDGKVVFEQFREIIRKVDILLGVFGQKPNSFLQDLKTIRIIRNKIDVNQVEELLSKRRQAREEKNFVQADEVRNTLASLGIEIRDTSEGQVWDIL

SEQ ID NO: 21 - protein, 494 aa, synthetic construct
  REGION 1..494: note = Cloned peptide
MHHHHHHGSMHLYNTMEKKKEPLIPIISGKLGIYVCGITAYDFSHIGHARSAIVFDILVRLLRYQGYDVTFIRNFTDIDDKIINRANKEGRSSKEVAEEFINAFHEDMDRLGVLNADIEPKATDYIPEMIECCQKLLEADKAYITASGDVYFRVRSFPDYGKLSGRTPDELRIGVRIVPSEEKEDPLDFVLWKAAKPGEPSWESPWGRGRPGWHIECSAMSEKCWPLPLDIHGGGIDLIFPHHENEIAQTESIVNKPLAKIWMHNGLVQVNSEKMSKSLGNFKIVRDILEAYLPETLRFFLLKKHYRSPIDFSFEGMNETERSQKRVYECIAEVDKALERKSWDSGGSSSSILAELDEQFSLFMSALEDDCNTAAGLGHLFNIIHIVRRALDDKALYSTTDGKVVFEQFREIIRKVDILLGVFGQKPNSFLQDLKTIRIIRNKIDVNQVEELLSKRRQAREEKNFVQADEVRNTLASLGIEIRDTSEGQVWDIL

SEQ ID NO: 22 - DNA, 1215 bp, Lawsonia intracellularis
  source 1..1215: mol_type = unassigned DNA
ttgatacagttttttgcttgtatttttaggggagaagatatgaccattgaaaaggggagatacttttttacctctgaatcagtaacagaaggtcatccagataaagttgctgatcaaatttctgatgctgttttagatacgttgttagagcaagatccacattctagagtagcttgtgaggctttagtttctactgggatggctattatcgctggggaaattacaacaaatggttatgcagatttgctaagtgtagttcgaaggaccatacagtccattggttataatagttctgagatgggttttgactggcagacatgtgcagttctttccacaattgataagcaatcattagatattgctcagggaattgatcgtgcaaaacctgaagagcaaggtgcaggagatcagggaatgatgtttggttttgcttgtagagaaactccaacgttaatgccagcacctatttattgggcacatcagctttctcaaaaattaacaaaagtacgaaaagatggagaagtgtccttttttcggccagatgggaaaacacaaatatcttttgagtattataatggggttcctgtaagaattaacaacgttgttgtttctacacaacatgccccttctgtatcacaaagtgaccttattgaagctgttaagagtaaagtgattcgtcctgtacttgaacctacaggaatgttcgatgagaaagaatgcgaaatatatattaatacaacaggacgttttgttgttggtggccctatgggtgattgtggtttgacaggaagaaaaattattcaagatacgtatggggggtcaggccatcatggcggtggagctttctcagggaaagatgcctcaaaagttgatcgttcaggagcatatatggctcgttatattgcaaaaaatgtagttggtactggattagcacaacgctgtgaagtacaaattgcttattgtattggtgtagcaaagcctgtttctgttttagtatcttctttaggtacaagcgatattcctgatgaagtcttaacaaaagctgtacttgaggtttttgatctaagaccttattttattacaaagcgtttagatttactaagaccaatttatagtaagacttcttgttatgggcattttggaagagaattacctgaatttacttgggaacaattagatgctgttaaagatttacaaacagctttaaaaatataa

SEQ ID NO: 23 - protein, 404 aa, Lawsonia intracellularis
MIQFFACIFRGEDMTIEKGRYFFTSESVTEGHPDKVADQISDAVLDTLLEQDPHSRVACEALVSTGMAIIAGEITTNGYADLLSVVRRTIQSIGYNSSEMGFDWQTCAVLSTIDKQSLDIAQGIDRAKPEEQGAGDQGMMFGFACRETPTLMPAPIYWAHQLSQKLTKVRKDGEVSFFRPDGKTQISFEYYNGVPVRINNVVVSTQHAPSVSQSDLIEAVKSKVIRPVLEPTGMFDEKECEIYINTTGRFVVGGPMGDCGLTGRKIIQDTYGGSGHHGGGAFSGKDASKVDRSGAYMARYIAKNVVGTGLAQRCEVQIAYCIGVAKPVSVLVSSLGTSDIPDEVLTKAVLEVFDLRPYFITKRLDLLRPIYSKTSCYGHFGRELPEFTWEQLDAVKDLQTALKI

SEQ ID NO: 24 - protein, 400 aa, synthetic construct
  REGION 1..400: note = Cloned peptide
MHHHHHHGSMTIEKGRYFFTSESVTEGHPDKVADQISDAVLDTLLEQDPHSRVACEALVSTGMAIIAGEITTNGYADLLSVVRRTIQSIGYNSSEMGFDWQTCAVLSTIDKQSLDIAQGIDRAKPEEQGAGDQGMMFGFACRETPTLMPAPIYWAHQLSQKLTKVRKDGEVSFFRPDGKTQISFEYYNGVPVRINNVVVSTQHAPSVSQSDLIEAVKSKVIRPVLEPTGMFDEKECEIYINTTGRFVVGGPMGDCGLTGRKIIQDTYGGSGHHGGGAFSGKDASKVDRSGAYMARYIAKNVVGTGLAQRCEVQIAYCIGVAKPVSVLVSSLGTSDIPDEVLTKAVLEVFDLRPYFITKRLDLLRPIYSKTSCYGHFGRELPEFTWEQLDAVKDLQTALKI

SEQ ID NO: 25 - DNA, 1092 bp, Lawsonia intracellularis
  source 1..1092: mol_type = unassigned DNA
ttggatatactacttccctttgaaaaaagacgtgaacagttacataaggttatgtctgaaagaaaactggatggacttttagtatatcatgctgctaatagatactatttatcaggttttgaattacataatcctcaatgtaatgaaagtgcaggctgtttaattatattagctaatggtaaagactggttatgtacagactcaaggtacactgaggcagcaaaacgtctatgggatgaggatttcatatatagttatggacacaacattccagaacaacttggattacttataaataaaatattaccaaaaggtagctgtattggttttgagtcaaaagtactttcagtaaatttttttgaagcttttgttgaacaattacaagatattacacctgtaagtgctgatggtatagttgaagatttaagagtaataaaagacgaacaagaagttcgattaatggaacaatcgtgtttattaaaccatcaacttttaacctgggttccatcaatattgcgtcctggagagagtgaagcttctgtttcttgggaaatagaattattttttagaaaacatggtgcaaatgaattagctttcccaagtattgttgcatctggaggtaatgcagcattacctcatgctattccaagttcagatacacaaattgagagtgaagagttagtacttgtggatgttggtgctaggctatatgattattgttcggatcaaacacggacattttgggttggagataatccctctaaacgttttcagcaaacattagcattagtacaggaggcacagcatagagctattaaagctattcagcctggagtgttagcaaaagatgtgtataacacagtttatacattttttatagaatatggggtagaaaaagcttttaagcataatcttggacatggagttggtcttgaagtgcatgaagcaccctcattagggccacgtagtgaaacaatccttaaacctgggatggttattactgttgagccaggcttatattatcctgagtggggaggagtccgttgggagcatatggtgcttgttacagaagatggtgcaaaagtattttaa

SEQ ID NO: 26 - protein, 363 aa, Lawsonia intracellularis
MDILLPFEKRREQLHKVMSERKLDGLLVYHAANRYYLSGFELHNPQCNESAGCLIILANGKDWLCTDSRYTEAAKRLWDEDFIYSYGHNIPEQLGLLINKILPKGSCIGFESKVLSVNFFEAFVEQLQDITPVSADGIVEDLRVIKDEQEVRLMEQSCLLNHQLLTWVPSILRPGESEASVSWEIELFFRKHGANELAFPSIVASGGNAALPHAIPSSDTQIESEELVLVDVGARLYDYCSDQTRTFWVGDNPSKRFQQTLALVQEAQHRAIKAIQPGVLAKDVYNTVYTFFIEYGVEKAFKHNLGHGVGLEVHEAPSLGPRSETILKPGMVITVEPGLYYPEWGGVRWEHMVLVTEDGAKVF

SEQ ID NO: 27 - protein, 372 aa, synthetic construct
  REGION 1..372: note = Cloned peptide
MHHHHHHGSMDILLPFEKRREQLHKVMSERKLDGLLVYHAANRYYLSGFELHNPQCNESAGCLIILANGKDWLCTDSRYTEAAKRLWDEDFIYSYGHNIPEQLGLLINKILPKGSCIGFESKVLSVNFFEAFVEQLQDITPVSADGIVEDLRVIKDEQEVRLMEQSCLLNHQLLTWVPSILRPGESEASVSWEIELFFRKHGANELAFPSIVASGGNAALPHAIPSSDTQIESEELVLVDVGARLYDYCSDQTRTFWVGDNPSKRFQQTLALVQEAQHRAIKAIQPGVLAKDVYNTVYTFFIEYGVEKAFKHNLGHGVGLEVHEAPSLGPRSETILKPGMVITVEPGLYYPEWGGVRWEHMVLVTEDGAKVF

SEQ ID NO: 28 - DNA, 1647 bp, Lawsonia intracellularis
  source 1..1647: mol_type = unassigned DNA
atggcttctaaagaaatcctttttgatgctaaagcccgtgaaaaactttcacgaggtgtagataaacttgcaaatgctgttaaagtaacacttggacctaaaggccgtaatgtcgttattgaaaagtcttttggttccccagttattacaaaagatggtgtatctgttgcaaaagaaattgaacttgaagataagtttgaaaatatgggcgctcaaatggttaaagaagtagcttccaaaactagcgatattgctggtgatggaactacaacagcaacagtccttgcacaagctatttatcgtgaaggtgtaaaacttgtagcagctggtcgtaatcctatggccattaaacgtggcatagataaagctgttgttgctgttactaaagaactaagcgacattacaaagcctactcgtgaccaaaaagaaatagctcaagttggaaccatttctgcaaactctgatacaacaataggtaatatcatagctgaagctatggctaaagttggaaaagaaggtgttatcacagttgaggaagctaaaggtcttgaaactacattagatgtggttgaaggaatgaagtttgaccgtggctacctctctccatactttgtaactaatcctgagaaaatggtttgtgaacttgataacccttatatcctttgtaatgagaaaaagattactagcatgaaagacatgctaccaatcttagaacaagttgctaaagtaaaccgtccactccttattattgctgaagacgtagaaggtgaagcacttgcaacacttgtagtcaataagctccgtggagcactccaagttgtagccgtaaaagctcctggttttggtgaacgccgtaaagctatgcttgaagatattgctatccttactggaggagaagcaatatttgaagatcgtggtataaagcttgaaaatgtaagcttgtcttctttaggaacagctaaacgtgtagttattgacaaagaaaatactactatcgttgatggtgctggaaaatcagaagatattaaagctcgagttaaacaaattcgtgcacaaattgaagaaacaagctcagattatgatcgtgaaaaacttcaagaacgtcttgcaaaacttgttggtggagtagctgttatccatgttggagctgctactgaaactgaaatgaaagagaagaaggatcgtgtagaagatgctctaaatgcaacaagagctgcggttgaagaaggtattgtccctggtggtggtactgcttttgtccgctccattaaagtccttgatgatattaaacctgctgatgatgatgaacttgctggacttaatatcatccgtcgttctcttgaagagcctttacgtcaaattgctgcaaatgctggctatgaaggttctattgttgtagaaaaagttcgtgaagcaaaagatggttttggatttaatgctgcatcaggagaatatgaagaccttattaaagctggtgtcattgatcctaaaaaagttacacgtattgcattacaaaatgcagcatcagtagcctccttacttctaactacagaatgcgctattgctgaaaaaccagaacctaaaaaagatatgcctatgcctggcggtggtatgggtggtatgggtggtatggacggtatgtactag

SEQ ID NO: 29 - protein, 548 aa, Lawsonia intracellularis
MASKEILFDAKAREKLSRGVDKLANAVKVTLGPKGRNVVIEKSFGSPVITKDGVSVAKEIELEDKFENMGAQMVKEVASKTSDIAGDGTTTATVLAQAIYREGVKLVAAGRNPMAIKRGIDKAVVAVTKELSDITKPTRDQKEIAQVGTISANSDTTIGNIIAEAMAKVGKEGVITVEEAKGLETTLDVVEGMKFDRGYLSPYFVTNPEKMVCELDNPYILCNEKKITSMKDMLPILEQVAKVNRPLLIIAEDVEGEALATLVVNKLRGALQVVAVKAPGFGERRKAMLEDIAILTGGEAIFEDRGIKLENVSLSSLGTAKRVVIDKENTTIVDGAGKSEDIKARVKQIRAQIEETSSDYDREKLQERLAKLVGGVAVIHVGAATETEMKEKKDRVEDALNATRAAVEEGIVPGGGTAFVRSIKVLDDIKPADDDELAGLNIIRRSLEEPLRQIAANAGYEGSIVVEKVREAKDGFGFNAASGEYEDLIKAGVIDPKKVTRIALQNAASVASLLLTTECAIAEKPEPKKDMPMPGGGMGGMGGMDGMY

SEQ ID NO: 30 - protein, 557 aa, synthetic construct
  REGION 1..557: note = Cloned peptide
MHHHHHHGSMASKEILFDAKAREKLSRGVDKLANAVKVTLGPKGRNVVIEKSFGSPVITKDGVSVAKEIELEDKFENMGAQMVKEVASKTSDIAGDGTTTATVLAQAIYREGVKLVAAGRNPMAIKRGIDKAVVAVTKELSDITKPTRDQKEIAQVGTISANSDTTIGNIIAEAMAKVGKEGVITVEEAKGLETTLDVVEGMKFDRGYLSPYFVTNPEKMVCELDNPYILCNEKKITSMKDMLPILEQVAKVNRPLLIIAEDVEGEALATLVVNKLRGALQVVAVKAPGFGERRKAMLEDIAILTGGEAIFEDRGIKLENVSLSSLGTAKRVVIDKENTTIVDGAGKSEDIKARVKQIRAQIEETSSDYDREKLQERLAKLVGGVAVIHVGAATETEMKEKKDRVEDALNATRAAVEEGIVPGGGTAFVRSIKVLDDIKPADDDELAGLNIIRRSLEEPLRQIAANAGYEGSIVVEKVREAKDGFGFNAASGEYEDLIKAGVIDPKKVTRIALQNAASVASLLLTTECAIAEKPEPKKDMPMPGGGMGGMGGMDGMY

SEQ ID NO: 31 - DNA, 630 bp, Lawsonia intracellularis
  source 1..630: mol_type = unassigned DNA
atggatgatatttttaatatgacagtccctattgttgttgaaaataacggaagaaatgaaagagcttatgatatttactcaagactactaaaagatcgtattgttcttcttggtacagaagtaaatgatcaagtagcatcccttatttgtgcacaacttttatttttagagtcacaggatccagaaaaagaaatttatttatatattaatagccctggaggttcagtaactgcaggtctcgcaatatatgatactatgaactatattacaccaaatgtagcaacggtttgtatggggcgtgccgcaagtatgggtgctttgttattagctgctggtgaaaaaaatatgcgttatgccttacctaatagccaggtaatgattcatcagccattaggcggttaccaaggacaagctacagatattgatatccatgccagagaaattttacgtatgcgtcaacggttaaatgaaattttgatggagcagacaggacagtcacttgaaaaggttgcacaagatactgagcgtgactattttatgacggcagaagatgctaaagcttatggacttattgataaagtcttggtttcccgaaaagatttagatatagagcatgaaaaaacagaataa

SEQ ID NO: 32 - protein, 209 aa, Lawsonia intracellularis
MDDIFNMTVPIVVENNGRNERAYDIYSRLLKDRIVLLGTEVNDQVASLICAQLLFLESQDPEKEIYLYINSPGGSVTAGLAIYDTMNYITPNVATVCMGRAASMGALLLAAGEKNMRYALPNSQVMIHQPLGGYQGQATDIDIHAREILRMRQRLNEILMEQTGQSLEKVAQDTERDYFMTAEDAKAYGLIDKVLVSRKDLDIEHEKTE

SEQ ID NO: 33 - protein, 220 aa, synthetic construct
  REGION 1..220: note = Cloned peptide
MHHHHHHGSEFMDDIFNMTVPIVVENNGRNERAYDIYSRLLKDRIVLLGTEVNDQVASLICAQLLFLESQDPEKEIYLYINSPGGSVTAGLAIYDTMNYITPNVATVCMGRAASMGALLLAAGEKNMRYALPNSQVMIHQPLGGYQGQATDIDIHAREILRMRQRLNEILMEQTGQSLEKVAQDTERDYFMTAEDAKAYGLIDKVLVSRKDLDIEHEKTE

SEQ ID NO: 34 - protein, 12 aa, synthetic construct
  REGION 1..12: note = HH2
VQLRIRVAVIRA

SEQ ID NO: 35 - protein, 12 aa, synthetic construct
  REGION 1..12: note = 1002
VQRWLIVWRIRK

SEQ ID NO: 36 - protein, 12 aa, synthetic construct
  REGION 1..12: note = 1018
VRLIVAVRIWRR

SEQ ID NO: 37 - protein, 13 aa, synthetic construct
  REGION 1..13: note = Indolicidin
ILPWKWPWWPWRR

SEQ ID NO: 38 - protein, 13 aa, synthetic construct
  REGION 1..13: note = HH111
ILKWKWPWWPWRR

SEQ ID NO: 39 - protein, 13 aa, synthetic construct
  REGION 1..13: note = HH113
ILPWKKPWWPWRR

SEQ ID NO: 40 - protein, 13 aa, synthetic construct
  REGION 1..13: note = HH970
ILKWKWPWWKWRR

SEQ ID NO: 41 - protein, 12 aa, synthetic construct
  REGION 1..12: note = HH1010
ILRWKWRWWRWR

SEQ ID NO: 42 - protein, 34 aa, synthetic construct
  REGION 1..34: note = Nisin Z
  SITE 2: note = misc_feature - Xaa = Dehydrobutyrine (Dhb)
  SITE 5: note = misc_feature - Xaa = Dehydroalanine (Dha)
  SITE 8: note = misc_feature - Xaa = 2-Aminobutyric acid (Abu)
  SITE 13: note = misc_feature - Xaa = 2-Aminobutyric acid (Abu)
  SITE 23: note = misc_feature - Xaa = 2-Aminobutyric acid (Abu)
  SITE 25: note = misc_feature - Xaa = 2-Aminobutyric acid (Abu)
  SITE 33: note = misc_feature - Xaa = Dehydroalanine (Dha)
IXAIXLAXPGAKXGALMGANMKXAXANASINVXL

SEQ ID NO: 43 - protein, 12 aa, synthetic construct
  REGION 1..12: note = JK1
VFLRRIRVIVIR

SEQ ID NO: 44 - protein, 11 aa, synthetic construct
  REGION 1..11: note = JK2
VFWRRIRVWVI

SEQ ID NO: 45 - protein, 12 aa, synthetic construct
  REGION 1..12: note = JK3
VQLRAIRVRVIR

SEQ ID NO: 46 - protein, 12 aa, synthetic construct
  REGION 1..12: note = JK4
VQLRRIRVWVIR

SEQ ID NO: 47 - protein, 12 aa, synthetic construct
  REGION 1..12: note = JK5
VQWRAIRVRVIR

SEQ ID NO: 48 - protein, 12 aa, synthetic construct
  REGION 1..12: note = JK6
VQWRRIRVWVIR

SEQ ID NO: 49 - DNA, 20 bp, synthetic construct
  misc_feature 1..20: note = CpG ODN 1826
  source 1..20: mol_type = other DNA
tccatgacgttcctgacgtt

SEQ ID NO: 50 - DNA, 20 bp, synthetic construct
  misc_feature 1..20: note = CpG ODN 2007
  source 1..20: mol_type = other DNA
tccatgacgttcctgacgtt

SEQ ID NO: 51 - DNA, 20 bp, synthetic construct
  misc_feature 1..20: note = CPG 7909 or 10103
  source 1..20: mol_type = other DNA
tccatgacgttcctgacgtt

SEQ ID NO: 52 - DNA, 21 bp, synthetic construct
  misc_feature 1..21: note = CpG 8954
  source 1..21: mol_type = other DNA
ggggacgacgtcgtggggggg

SEQ ID NO: 53 - DNA, 21 bp, synthetic construct
  misc_feature 1..21: note = CpG 2395 or CpG 10101
  source 1..21: mol_type = other DNA
ggggacgacgtcgtggggggg

SEQ ID NO: 54 - DNA, 32 bp, synthetic construct
  misc_feature 1..32: note = Non-CPG ISS
  source 1..32: mol_type = other DNA
aaaaaaggtacctaaatagtatgtttctgaaa

SEQ ID NO: 55 - DNA, 37 bp, synthetic construct
  misc_feature 1..37: note = primer
  source 1..37: mol_type = other DNA
gacggatcctctcttgtcattaataacaacctgatgg

SEQ ID NO: 56 - DNA, 37 bp, synthetic construct; identical to SEQ ID NO: 55
  misc_feature 1..37: note = primer
  source 1..37: mol_type = other DNA

SEQ ID NO: 57 - DNA, 32 bp, synthetic construct
  misc_feature 1..32: note = primer
  source 1..32: mol_type = other DNA
gagggatccgctaatgttagtggaatccctgc

SEQ ID NO: 58 - DNA, 32 bp, synthetic construct; identical to SEQ ID NO: 57
  misc_feature 1..32: note = primer
  source 1..32: mol_type = other DNA

SEQ ID NO: 59 - DNA, 29 bp, synthetic construct
  misc_feature 1..29: note = primer
  source 1..29: mol_type = other DNA
tcccatggctgaggctgttgaacactttg

SEQ ID NO: 60 - DNA, 33 bp, synthetic construct
  misc_feature 1..33: note = primer
  source 1..33: mol_type = other DNA
ggctcgagttagaatctataagtagctcctacc

SEQ ID NO: 61 - DNA, 32 bp, synthetic construct
  misc_feature 1..32: note = primer
  source 1..32: mol_type = other DNA
cgccatggacagtgatgaggaccttagtacag

SEQ ID NO: 62 - DNA, 30 bp, synthetic construct
  misc_feature 1..30: note = primer
  source 1..30: mol_type = other DNA
agctcgagtaggaatccaccactgatcaag

SEQ ID NO: 63 - DNA, 43 bp, synthetic construct
  misc_feature 1..43: note = primer
  source 1..43: mol_type = other DNA
gagggatccatgttgttatatataaataaagaacacattattg

SEQ ID NO: 64 - DNA, 40 bp, synthetic construct
  misc_feature 1..40: note = primer
  source 1..40: mol_type = other DNA
gagctcgagttatacttcttctgtataataattttgttca

SEQ ID NO: 65 - DNA, 41 bp, synthetic construct
  misc_feature 1..41: note = primer
  source 1..41: mol_type = other DNA
gagggatccatgttcaaaaaaatatatgttttttatatcac

SEQ ID NO: 66 - DNA, 39 bp, synthetic construct
  misc_feature 1..39: note = primer
  source 1..39: mol_type = other DNA
gagctcgagttattcattagggacaataataggtgttac

SEQ ID NO: 67 - DNA, 36 bp, synthetic construct
  misc_feature 1..36: note = primer
  source 1..36: mol_type = other DNA
gagggatccatgcatctatataatactatggaaaag

SEQ ID NO: 68 - DNA, 33 bp, synthetic construct
  misc_feature 1..33: note = primer
  source 1..33: mol_type = other DNA
gagctcgagttataaaatatcccacacctgacc

SEQ ID NO: 69 - DNA, 33 bp, synthetic construct; identical to SEQ ID NO: 68
  misc_feature 1..33: note = primer
  source 1..33: mol_type = other DNA

SEQ ID NO: 70 - DNA, 36 bp, synthetic construct
  misc_feature 1..36: note = primer
  source 1..36: mol_type = other DNA
cgcctcgagttatatttttaaagctgtttgtaaatc

SEQ ID NO: 71 - DNA, 40 bp, synthetic construct
  misc_feature 1..40: note = primer
  source 1..40: mol_type = other DNA
gagggatccatggatatactacttccctttgaaaaaagac

SEQ ID NO: 72 - DNA, 40 bp, synthetic construct; identical to SEQ ID NO: 71
  misc_feature 1..40: note = primer
  source 1..40: mol_type = other DNA

SEQ ID NO: 73 - DNA, 40 bp, synthetic construct; identical to SEQ ID NO: 71
  misc_feature 1..40: note = primer
  source 1..40: mol_type = other DNA

SEQ ID NO: 74 - DNA, 35 bp, synthetic construct
  misc_feature 1..35: note = primer
  source 1..35: mol_type = other DNA
gaaggcggccgctagtacataccgtccataccacc

SEQ ID NO: 75 - DNA, 36 bp, synthetic construct
  misc_feature 1..36: note = primer
  source 1..36: mol_type = other DNA
gaggaattcatggatgatatttttaatatgacagtc

SEQ ID NO: 76 - DNA, 36 bp, synthetic construct; identical to SEQ ID NO: 75
  misc_feature 1..36: note = primer
  source 1..36: mol_type = other DNA

SEQ ID NO: 77 - DNA, 21 bp, synthetic construct
  misc_feature 1..21: note = primer
  source 1..21: mol_type = other DNA
ggttagtcgttgcccatgata

SEQ ID NO: 78 - DNA, 21 bp, synthetic construct
  misc_feature 1..21: note = primer
  source 1..21: mol_type = other DNA
ctgcgatatgctcccatagtt

(19) United States
(12) Patent Application Publication
(10) Pub. No.: US 2024/0066111 A1
(43) Pub. Date: Feb. 29, 2024

(54) LAWSONIA INTRACELLULARIS COMPOSITIONS AND METHODS OF USING THE SAME

(71) Applicant: University of Saskatchewan, Saskatoon (CA)

(72) Inventors: Heather Lynne Wilson, Saskatoon (CA); Milan Obradovic, Saskatoon (CA)

(21) Appl. No.: 18/153,483

(22) Filed: Jan. 12, 2023

Related U.S. Application Data
(63) Continuation of application No. 17/434,551, filed on Aug. 27, 2021, now abandoned.
(60) Provisional application No. 62/811,974, filed on Feb. 28, 2019.

(51) Int. Cl. A61K 39/02; A61K 9/107; A61P 31/04; C07K 14/195; C12N 15/70

(52) U.S. Cl. CPC A61K 39/105; A61K 9/107; A61P 31/04; C07K 14/195; C12N 15/70; A61K 2039/552

(57) ABSTRACT

L intracellularis antigens for use in subunit vaccine compositions to elicit immune responses against L intracellularis infections such as proliferative enteropathy (PE) are described, as well as polynucleotides encoding therefor. Also described are methods for treating and preventing L intracellularis infections.

### CROSS-REFERENCE TO RELATED APPLICATIONS

This is a continuation of U.S. patent application Ser. No. 17/434,551, filed Aug. 27, 2021, which is a National Stage Entry under 35 U.S.C. 371 of International Patent Application No. PCT/CA2020/050277, filed Feb. 28, 2020, which claims priority to U.S. Provisional Patent Application No. 62/811,974, filed Feb. 28, 2019. These applications are incorporated by reference herein.

### REFERENCE TO AN ELECTRONIC SEQUENCE LISTING

The contents of the electronic sequence listing (GOW.11.C1_Seq-List.xml., Size: 118,275 bytes; and Date of Creation: Jan. 12, 2023) is herein incorporated by reference in its entirety.

### TECHNICAL FIELD

The present invention relates generally to bacterial pathogens. In particular, the invention pertains to Lawsonia intracellularis immunogenic compositions and methods of treating, preventing and/or diagnosing Lawsonia-related disorders, such as proliferative enteropathy.

### BACKGROUND

Lawsonia intracellularis is an obligate intracellular Gram negative bacterium with fastidious microaerophilic growth requirements. It is the causative agent of proliferative enteropathy (PE) which is an economically important disease in pigs and is also found in other mammals including non-human primates, horses, rabbits, and birds such as emus and ostriches (Kroll et al., Animal Health Res. Rev. (2005) 6:173-197). L. intracellularis infects enterocytes in the distal ileum and jejunum and cannot replicate outside eukaryotic cells. Attachment and invasion of bacteria to enterocytes is an important step in bacterial infection but the mechanism by which these bacteria interact with the host cells has not yet been determined (Vannucci et al., Vet. Pathol. (2014) 51:465-477).

The Type III secretion system (T3SS) is a common secretion system found in many enteroinvasive pathogens and plays a role in invasion and suppression of innate defenses. Proteins that are part of this system have been detected in three Lawsonia isolates (Alberdi et al., Vet. Microbiol. (2009) 139:298-303). These T3SS proteins and other uncharacterized bacterial proteins that facilitate contact with enterocytes are expressed on the cell surface and are therefore accessible to the host immune system. However, establishing the importance of these proteins for attachment has been hampered by the obligate intracellular growth requirement of L. intracellularis, as well as by difficulties of removing eukaryotic host cell proteins from sample preparations.

L. intracellularis autotransporter protein (LatA) has been detected using mass spectrometry (MS) and bioinformatics (Watson et al., Clin. and Vaccine Immunol. (2011) 18:1282-1287). Additionally, shotgun proteomic analysis has been used to identify 19 unique proteins during in vitro infection, two of which proteins, LI0841 and LI0902, were shown to have antigenic properties (Watson et al., Vet. Microbiol. (2014) 174:448-455).

Two-dimensional gel electrophoresis (2DE) is an efficient analytical tool for separation of complex protein mixtures from tissue, mammalian and bacterial cells, and secretions (Magdeldin et al., Clin. Proteomics (2014) 11:16). 2DE is a robust and confident technique with the advantage of being compatible with other biochemical techniques (Rabilloud et al., J. Proteomics (2010) 73:2064-2077). For instance, 2DE, coupled with Western blot (WB) and Mass Spectrometry (MS), has been used to detect bacterial antigens recognized by the human immune system (Lahner et al., International J. Med. Microbiol. (2011) 301:125-132; Havlsová et al., Proteomics (2005) 5:2090-2103), cancer cell antigens (Kellner et al., Proteomics (2002) 2:1743-1751), and fungal antigens (Pitarch et al., Electophoresis (1999) 20:1001-1010).

Despite these techniques for identifying potential L. intracellularis antigens, vaccine development has been lacking. An avirulent live vaccine has been developed (Kroll et al., Am. J. Vet. Res. (2004) 65:559-565). However, animals administered the vaccine still shed bacteria in great numbers, thus presenting danger of infection to other animals. A vaccine containing inactivated, whole cell bacteria has also been developed (Roerink et al., Vaccine (2018) 36:1500-1508). However, because the commercially available L. intracellularis vaccines are dependent on the growth of this obligate intracellular pathogen, production is limited and time-consuming.

It is clear that the identification of antigens for use in subunit vaccine compositions for treating and/or preventing Lawsonia infection, or in diagnostics for detecting Lawsonia infection, is needed.

### SUMMARY OF THE INVENTION

The inventors herein have successfully identified and characterized L. intracellularis antigens for use in subunit vaccine compositions by combining the separation power of two-dimensional gel electrophoresis (2DE) with Western blot (WB) analysis and Mass Spectrometry (MS). Downstream applications such as WB and MS add to the analytical power of 2DE and allow efficient identification of targeted proteins.

L. intracellularis proteins were identified and further bioinformatics analysis and flow cytometry assays indicated several of the proteins were likely vaccine antigens. Genes coding for proteins were cloned and expressed, and the corresponding recombinant proteins were purified. Proteins described herein are shown to be immunogenic based on porcine hyperimmune sera and vaccine trial data. Rabbit immune sera generated against a vaccine strain of L. intracellularis and sera specific for recombinant proteins showed an inhibitory effect on the attachment and penetration of live, avirulent L. intracellularis, indicating that each protein tested was a neutralizing antibody target useful in subunit vaccine formulations.

Subunit vaccines may allow recombinant antigen production to be performed in host cells, such as E. coli, which provides a safe, rapid and inexpensive alternative to vaccines that require growth, attenuation and inactivation of L. intracellularis. L. intracellularis protein LI0710 (Flagellin) has both adjuvant and antigenic properties that induce a specific immune response in intestinal mucosa of mice. Thus, vaccines including this antigen may not require additional adjuvants to provide protection against this intestinal pathogen.

Accordingly, the present invention provides L. intracellularis subunit compositions for the treatment and/or prevention of Lawsonia infections, such as proliferative enteropathy (PE), in pigs and other susceptible animals. Subunit vaccines, including immunogens and mixtures of immunogens derived from L. intracellularis isolates, are used to provide protection against subsequent infection and/or to diagnose infection. The present invention thus provides a commercially useful method of treating, preventing and/or diagnosing L. intracellularis infection in swine and other mammals.

In one embodiment, an immunogenic, subunit composition is provided. The composition comprises at least one isolated, immunogenic L. intracellularis protein selected from an LI0710, an LI0649, an LI0169, an LI1153, an LI0786, an LI1171, an LI0608, an LI0726, an LI0823, an LI0625, an LI0794, an immunogenic fragment thereof, an immunogenic variant thereof, or the corresponding protein from another L. intracellularis strain or isolate, and a pharmaceutically acceptable excipient.

In certain embodiments, the L. intracellularis protein(s) is selected from one or more proteins comprising the amino acid sequence of SEQ ID NOs: 2, 5, 8, 11, 14, 17, 20, 23, 26, 29 and 32, an immunogenic fragment thereof, or an immunogenic fragment or variant thereof.

In additional embodiments, the L. intracellularis immunogenic protein comprises the amino acid sequence of SEQ ID NOs: 2, 5, 8, 11, 14, 17, 20, 23, 26, 29 or 32, with a deletion of all or part of a transmembrane binding domain or a native signal sequence, if present.

In further embodiments, the L. intracellularis immunogenic protein comprises an amino acid sequence with at least 90% sequence identity to the amino acid sequence of SEQ ID NOs: 2, 5, 8, 11, 14, 17, 20, 23, 26, 29 or 32.

In certain embodiments, the L. intracellularis immunogenic protein comprises the amino acid sequence of amino acids 2-293 of SEQ ID NO: 2; the amino acid sequence of amino acids 31-851 of SEQ ID NO: 5; the amino acid sequence of amino acids 43-552 of SEQ ID NO: 8, the amino acid sequence of amino acids 5-398 of SEQ ID NO: 11; the amino acid sequence of amino acids 1-383 of SEQ ID NO: 14; the amino acid sequence of amino acids 1-562 of SEQ ID NO: 17; the amino acid sequence of amino acids 1-485 of SEQ ID NO: 20; the amino acid sequence of amino acids 1-404 of SEQ ID NO: 23; the amino acid sequence of amino acids 1-363 of SEQ ID NO: 26; the amino acid sequence of amino acids 1-548 of SEQ ID NO: 29; and/or the amino acid sequence of amino acids 1-209 of SEQ ID NO: 32.

In yet additional embodiments, the immunogenic composition comprises two or more isolated immunogenic L. intracellularis proteins. In certain embodiments, the two or more proteins are provided as a fusion protein.

In further embodiments, the immunogenic composition further comprises an immunological adjuvant such as, but not limited to, an oil-in-water emulsion adjuvant. In some embodiments, the immunological adjuvant is alum.

In other embodiments, the immunological adjuvant comprises (a) a polyphosphazene; (b) a poly(I:C) or a CpG oligonucleotide; and (c) a host defense peptide, and can be in the form of a microparticle. In certain embodiments, the polyphosphazene is PCEP and the host defense peptide is peptide 1002. In some embodiments, the polyphosphazene is a linear or cyclic polyphosphazene.

In some embodiments, the immunogenic composition further comprises a mucoadhesive lipidic carrier system with one or more cationic lipids selected from: 1,2-dioleoyl-3-trimethylammonium-propane (DOTAP); 3β-[N—(N′,N′-dimethylaminoethane)-carbamoyl] (DC); dimethyldioctadecylammonium (DDA); octadecylamine (SA); dimethyldioctadecylammonium bromide (DDAB); 1,2-dioleoyl-sn-glycerol-3-phosphoethanolamine (DOPE); egg L-α-phosphatidylcholine (EPC); cholesterol (Chol); distearoylphosphatidylcholine (DSPC); 1,2-dimyristoyl-3-trimethylammonium-propane (DMTAP); dimyristoylphosphatidylcholine (DMPC); or ceramide carbamoyl-spermine (CCS).

In further embodiments, a recombinant vector is provided. The recombinant vector comprises (a) a DNA molecule encoding an immunogenic L. intracellularis protein selected from an LI0710, an LI0649, an LI0169, an LI1153, an LI0786, an LI1171, an LI0608, an LI0726, an LI0823, an LI0625, an LI0794, an immunogenic fragment thereof, an immunogenic variant thereof, or the corresponding protein from another L. intracellularis strain or isolate; and (b) control elements that are operably linked to the DNA molecule whereby a coding sequence in the molecule can be transcribed and translated in a host cell.

In additional embodiments, a host cell transformed with the recombinant vector is provided. In certain embodiments, the host cell is an E. coli cell.

In yet further embodiments, a method of producing a L. intracellularis protein is provided. The method comprises: (a) providing a population of host cells as above; and (b) culturing the population of cells under conditions whereby the protein encoded by the DNA molecule present in the recombinant vector is expressed.

In further embodiments, a method of treating or preventing a L. intracellularis infection in a vertebrate subject is provided that comprises administering a therapeutic amount of any of the compositions described herein to the subject. In certain embodiments, the subject is a porcine or equine subject. In additional embodiments, the L. intracellularis infection comprises a proliferative enteropathy.

These and other embodiments of the subject invention will readily occur to those of skill in the art in view of the disclosure herein.

### BRIEF DESCRIPTION OF THE FIGURES

FIGS. 1A, 1B and 1C show the DNA sequence (SEQ ID NO: 1, FIG. 1A), the amino acid sequence (SEQ ID NO: 2, FIG. 1B; NCBI no. CAJ54764) and the amino acid sequence of the cloned gene product (SEQ ID NO: 3, FIG. 1C) of LI0710 from L. intracellularis isolate PHE/MN1-00. FIG. 1B shows the amino acid sequences predicted from the DNA sequence and FIG. 1C shows the cloned sequence (SEQ ID NO: 3).

FIGS. 2A, 2B and 2C show the DNA sequence (SEQ ID NO: 4, FIG. 2A), the amino acid sequence (SEQ ID NO: 5, FIG. 2B; NCBI no. CAJ54703) and the amino acid sequence of the cloned gene product (SEQ ID NO: 6, FIG. 2C) of LI0649 from L. intracellularis isolate PHE/MN1-00. The N-terminal region including a transmembrane domain is deleted from the recombinant molecule described in the examples, in FIG. 2B. FIG. 2B shows the amino acid sequences predicted from the DNA sequence and FIG. 2C shows the cloned sequence (SEQ ID NO: 6). The N-terminal region including a transmembrane domain from FIG. 2B is deleted from the recombinant molecule described in the examples, which is depicted in FIG. 2C.

FIGS. 3A, 3B and 3C show the DNA sequence (SEQ ID NO: 7, FIG. 3A), the amino acid sequence (SEQ ID NO: 8, FIG. 3B; NCBI no. CAJ54225) and the amino acid sequence of the cloned gene product (SEQ ID NO: 9, FIG. 3C) of LI0169 from L. intracellularis isolate PHE/MN1-00. FIG. 3B shows the amino acid sequences predicted from the DNA sequence and FIG. 3C shows the cloned sequence (SEQ ID NO: 9).

FIGS. 4A, 4B and 4C show the DNA sequence (SEQ ID NO: 10, FIG. 4A), the amino acid sequence (SEQ ID NO: 11, FIG. 4B; NCBI no. CAJ55207) and the amino acid sequence of the cloned gene product (SEQ ID NO: 12, FIG. 4C) of LI1153 from L. intracellularis isolate PHE/MN1-00. FIG. 4B shows the amino acid sequences predicted from the DNA sequence and FIG. 4C shows the cloned sequence (SEQ ID NO: 12).

FIGS. 5A, 5B, and 5C show the DNA sequence (SEQ ID NO: 13, FIG. 5A), the amino acid sequence (SEQ ID NO: 14, FIG. 5B; NCBI no. CAJ54840) and the amino acid sequence of the cloned gene product (SEQ ID NO: 15, FIG. 5C) of LI0786 from L. intracellularis isolate PHE/MN1-00. FIG. 5B shows the amino acid sequences predicted from the DNA sequence and FIG. 5C shows the cloned sequence (SEQ ID NO: 15).

FIGS. 6A, 6B, and 6C show the DNA sequence (SEQ ID NO: 16, FIG. 6A), the amino acid sequence (SEQ ID NO: 17, FIG. 6B; NCBI no. CAJ55225) and the amino acid sequence of the cloned gene product (SEQ ID NO: 18, FIG. 6C) of LI1171 from L. intracellularis isolate PHE/MN1-00. FIG. 6B shows the amino acid sequences predicted from the DNA sequence and FIG. 6C shows the cloned sequence (SEQ ID NO: 18).

FIGS. 7A, 7B, and 7C show the DNA sequence (SEQ ID NO: 19, FIG. 7A), the amino acid sequence (SEQ ID NO: 20, FIG. 7B; NCBI no. CAJ54662) and the amino acid sequence of the cloned gene product (SEQ ID NO: 21, FIG. 7C) of LI0608 from L. intracellularis isolate PHE/MN1-00. FIG. 7B shows the amino acid sequences predicted from the DNA sequence and FIG. 7C shows the cloned sequence (SEQ ID NO: 21).

FIGS. 8A, 8B and 8C show the DNA sequence (SEQ ID NO: 22, FIG. 8A), the amino acid sequence (SEQ ID NO: 23, FIG. 8B; NCBI no. CAJ54780) and the amino acid sequence of the cloned gene product (SEQ ID NO: 24, FIG. 8C) of LI0726 from L. intracellularis isolate PHE/MN1-00. FIG. 8B shows the amino acid sequences predicted from the DNA sequence and FIG. 8C shows the cloned sequence (SEQ ID NO: 24).

FIGS. 9A, 9B, and 9C show the DNA sequence (SEQ ID NO: 25, FIG. 9A), the amino acid sequence (SEQ ID NO: 26, FIG. 9B; NCBI no. CAJ54877) and the amino acid sequence of the cloned gene product (SEQ ID NO: 27, FIG. 9C) of LI0823 from L. intracellularis isolate PHE/MN1-00. FIG. 9B shows the amino acid sequences predicted from the DNA sequence and FIG. 9C shows the cloned sequence (SEQ ID NO: 27).

FIGS. 10A, 10B and 10C show the DNA sequence (SEQ ID NO: 28, FIG. 10A), the amino acid sequence (SEQ ID NO: 29, FIG. 10B; NCBI no. CAJ54679) and the amino acid sequence of the cloned gene product (SEQ ID NO: 30, FIG. 10C) of LI0625 from L. intracellularis isolate PHE/MN1-00. FIG. 10B shows the amino acid sequences predicted from the DNA sequence and FIG. 10C shows the cloned sequence (SEQ ID NO: 30).

FIGS. 11A, 11B and 11C show the DNA sequence (SEQ ID NO: 31, FIG. 11A), the amino acid sequence (SEQ ID NO: 32, FIG. 11B; NCBI no. CAJ54848) and the amino acid sequence of the cloned gene product (SEQ ID NO: 33, FIG. 11C) of LI0794 from L. intracellularis isolate PHE/MN1-00. FIG. 11B shows the amino acid sequences predicted from the DNA sequence and FIG. 11C shows the cloned sequence (SEQ ID NO: 33).

FIGS. 12A, 12B and 12C show the inhibitory effect of rabbit sera on CFSE-labelled avirulent L. intracellularis penetration in IPEC-1 cells, as described in the examples: negative control sera (sera obtained prior to immunization and pooled), anti-L. intracellularis sera (serum from rabbits immunized with whole avirulent bacteria), sera from rabbits immunized with recombinant proteins: anti-rLI0169, anti-rLI0649, anti-rLI0710 (rFliC), anti-rLI1153; serum concentrations used in assay 500 μg/mL (FIG. 12A); 1000 μg/mL (FIG. 12B) and 2000 μg/mL (FIG. 12C). All sera were cleared from antibodies against LPS. Percent inhibition=(1−% of fluorescence of CFSE bacteria incubated with serum/% of fluorescence of CFSE bacteria (control))×100. Data presented for 4 biological replicates. The bar shows standard deviation of mean value of 4 biological replicates. (***) p<0.001, (**) p<0.01 and (*) p<0.05, (ns) not significant.

FIGS. 13A-D show intramuscular vaccination with rLI0710 vaccines formulated with VIDO-Triple Adjuvant (See, below) results in antigen-specific systemic and intestinal antibody response. VIDO Triple Adjuvant is comprised of poly IC, host defense peptide 1002, and polyphosphazene in a 1:2:1 ratio. Weaner piglets were immunized by the intramuscular route with 300 μg each of rLI0710 formulated with 300 μg:600 μg:300 μg polyphosphazene (VIDO Triple Adjuvant. They received booster doses 17 days later and 32 days later. Control animals were immunized by the same route and on the same days with VIDO Triple Adjuvant (n=4). Piglets were euthanized on day 46. Serum anti-rLI0710 IgG titres were quantified. FIG. 13A shows a schematic representation of an animal trial that was performed to show evidence that rLI0710 formulated with VIDO Triple Adjuvant and administered via the intramuscular route promotes a measurable humoral immune response. FIG. 13B shows serum anti-rLI0710 IgG titres in intramuscular vaccinated versus control animals. FIG. 13C and FIG. 13D show anti-rLI0710 IgA titres in ileum and jejunum scrapings, respectively, from intramuscular vaccinated versus control animals. Each symbol represents an individual animal and the horizontal bars showing the median value of each column. Statistical comparisons relative to control animals, P<0.05 (*).

FIGS. 14A-K show intramuscular vaccination with subunit vaccines formulated with EMULSIGEN® results in antigen-specific humoral response. FIG. 14A is a schematic representation of an immunization, bleeding and challenge animal trial with recombinant L. intracellularis proteins that promote a measurable immune response and/or protection against infectious L. intracellularis. Weaner piglets were immunized with 50 μg each of rLI0710, rLI0625 and rLI0169 with EMULSIGEN® (Group 1, n=8), 50 μg of rLI0794, 50 μg rLI0726 and 25 μg rLI0786 with EMULSIGEN® (Group 2, n=8), challenge control group administered EMULSIGEN® alone (Group 3, n=7) and unchallenged control group administered EMULSIGEN® alone (Group 4, n=7). Each vaccine was 1.4 mL total volume with 700 μL consisting of adjuvant and 700 μL antigen or saline buffer. Pigs were fasted overnight then challenged on day 27 with 1.9×108 pathogenic L. intracellularis in 40 mL of gut mucosa from previously infected pigs. Sera were collected at day 0, 14 and 27 and jejunum and ileum were scraped on day 48. FIGS. 14B-D shows anti-rLI0710 (FIG. 14B), anti-rLI0625 (FIG. 14C) and anti-rLI0794 IgG (FIG. 14D) in sera over day 0, 14 and 27 from intramuscular vaccinated and control pigs. FIGS. 14E-G and FIGS. 14H-J show anti-rLI0710 (FIG. 14E, FIG. 14H), anti-rLI0625 (FIG. 14F, FIG. 14I) and anti-rLI0794 (FIG. 14G, FIG. 14J) IgA, from jejunum and ileum mucosal scrapings respectively, from vaccinated and control pigs at day 48. FIG. 14K shows weights of piglets taken in the first week of the trial, pre-challenge and carcass weight post-challenge. Each symbol represents an individual animal and the horizontal bars showing the median value of each column. Statistical comparisons relative to day 0 within each treatment group are shown with statistical significance, P<0.05, P<0.01 (**) and P<0.001 (***).

FIGS. 15A-B show intrauterine and intrauterine/intramuscular immunization of recombinant antigen formulated with VIDO Triple Adjuvant induced cell mediated immunity recall response. Gilts were immunized with 400 μg rLI0710 antigen formulated with VIDO Triple Adjuvant (1.2 mg poly IC, 2.4 mg host defense peptide, 1.2 mg polyphosphazene per dose) into the uterus during breeding with killed semen. For the second and third booster vaccines, gilts were bred with killed (2nd dose) and live (3rd dose) semen each with VIDO Triple Adjuvant. At the time of the 2nd and 3rd doses, gilts were immunized with rLI0710 plus (400 μg polyIC, 800 μg host defense peptide and 400 μg polyphosphazene per dose. Control gilts were administered killed or live semen (without VIDO Triple Adjuvant) by the intrauterine or intramuscular route. FIG. 15A is a schematic representation of an animal trial that was performed to show evidence that rLI0710 formulated with VIDO Triple Adjuvant and administered to a mucosal and intramuscular route promotes cell-mediated immunity. FIG. 15B shows IFNγ production quantified after peripheral blood mononuclear immune cells (PBMCs) were collected on approximately day 38 after the last immunization/breeding with live semen. PBMCs were restimulated with 2 μg/mL rLI0710. Each symbol represents an individual animal and the horizontal bars show the median value of each column. Statistical comparisons are made relative to mock-immunized animals within each treatment group and marked with statistical significance, P<0.001 (***)

### DETAILED DESCRIPTION OF THE INVENTION

The practice of the present invention will employ, unless otherwise indicated, conventional methods of microbiology, virology, chemistry, biochemistry, recombinant DNA techniques and immunology, within the skill of the art. Such techniques are explained fully in the literature. See, e.g., Fundamental Virology, Current Edition, vol. I & II (B. N. Fields and D. M. Knipe, eds.); Handbook of Experimental Immunology, Vols. I-IV (D. M. Weir and C. C. Blackwell eds., Blackwell Scientific Publications); T. E. Creighton, Proteins: Structures and Molecular Properties (W.H. Freeman and Company); A. L. Lehninger, Biochemistry (Worth Publishers, Inc., current edition); Sambrook, et al., Molecular Cloning: A Laboratory Manual (current edition); Methods In Enzymology (S. Colowick and N. Kaplan eds., Academic Press, Inc.).

All publications, patents and patent applications cited herein, whether supra or infra, are hereby incorporated by reference in their entireties.

The following amino acid abbreviations are used throughout the text:

| | |
|---|---|
| Alanine: Ala (A) | Arginine: Arg (R) |
| Asparagine: Asn (N) | Aspartic acid: Asp (D) |
| Cysteine: Cys (C) | Glutamine: Gln (Q) |
| Glutamic acid: Glu (E) | Glycine: Gly (G) |
| Histidine: His (H) | Isoleucine: Ile (I) |
| Leucine: Leu (L) | Lysine: Lys (K) |
| Methionine: Met (M) | Phenylalanine: Phe (F) |
| Proline: Pro (P) | Serine: Ser (S) |
| Threonine: Thr (T) | Tryptophan: Trp (W) |
| Tyrosine: Tyr (Y) | Valine: Val (V) |

### 1. DEFINITIONS

In describing the present invention, the following terms will be employed, and are intended to be defined as indicated below.

It must be noted that, as used in this specification and the appended claims, the singular forms “a”, “an” and “the” include plural referents unless the content clearly dictates otherwise. Thus, for example, reference to “an antigen” includes a mixture of two or more such antigens, and the like.

Lawsonia intracellularis is an obligate, intracellular Protobacterium that infects a wide range of mammalian and avian species including, without limitation, pigs, horses, primates, dogs, rats, guinea pigs, rabbits and hamsters. The bacterium infects the gastrointestinal tract, with a specific tropism for the terminal ileum, and causes proliferation of intestinal crypt lining cells (enterocytes), resulting in hyperplasia of the mucosal wall and a number of associated disorders, described further herein. L. intracellularis can also affect the jejunum and in some cases, the colon. The term “L. intracellularis” intends any subspecies, strain or isolate of the organism which is capable of causing disease.

The term “derived from” is used herein to identify the original source of a molecule but is not meant to limit the method by which the molecule is made which can be, for example, by chemical synthesis or recombinant means.

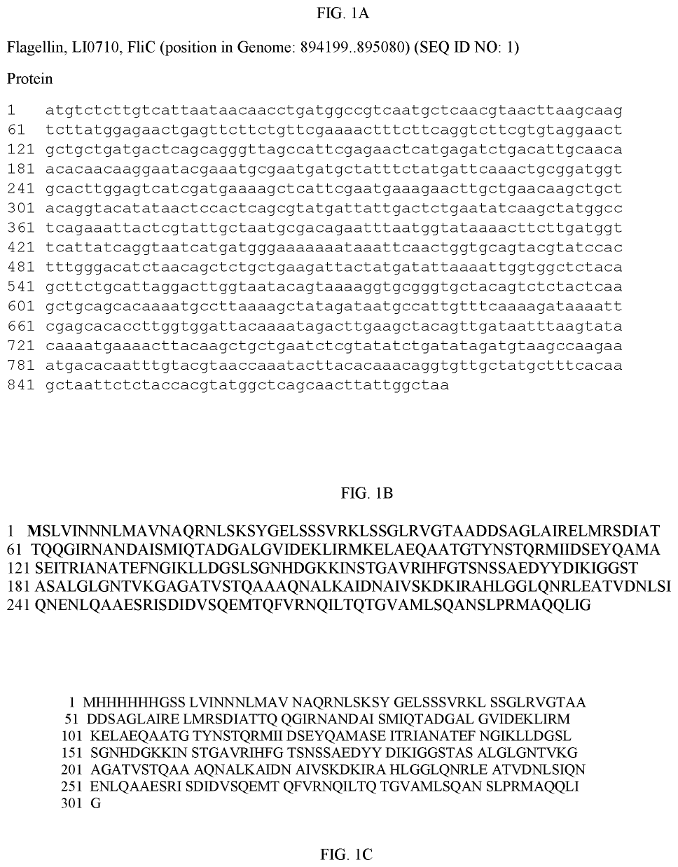

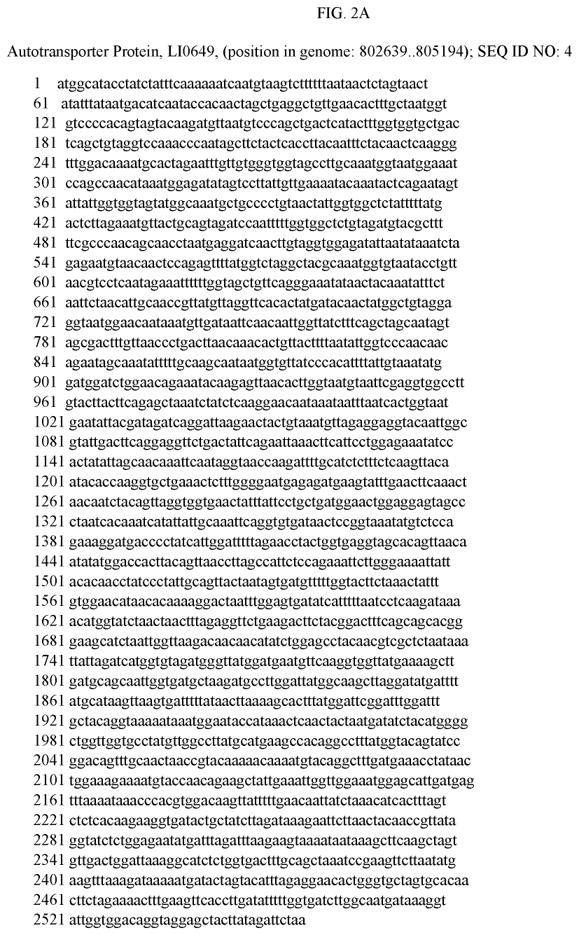

A “L. intracellularis molecule” is a molecule derived from the bacterium, including, without limitation, polypeptide, protein, antigen, polynucleotide, oligonucleotide, and nucleic acid molecules from any of the various L. intracellularis subspecies, strains, or isolates. The molecule need not be physically derived from the particular bacterium in question, but may be synthetically or recombinantly produced. Nucleic acid and polypeptide sequences from a number of L. intracellularis isolates are known and/or described herein. Representative L. intracellularis proteins, and polynucleotides encoding the proteins, for use in treating and/or preventing infection, such as PE, are presented in Tables 1 and 2, and FIGS. 1-11 herein. Additional representative sequences found in isolates from various mammals are listed in the National Center for Biotechnology Information (NCBI) database. See, also, Nishikawa et al., Microbiol. Resourc. Announc. (2018) Sep. 6:7(9); Sait et al., Genome Announc. (2013) January-February: 1(1); Mirajkar et al., Genome Announc. (2017) May 11:5(19). However, an L. intracellularis molecule, such as an antigen, as defined herein, is not limited to those shown and described in Tables 1 and 2, and FIGS. 1-11, as various isolates are known and variations in sequences may occur between them. Thus, a “L. intracellularis” molecule as defined herein intends a molecule from an L. intracellularis isolate or strain that corresponds to the particular L. intracellularis source molecule.

By “Lawsonia disease or disorder” is meant a disease or disorder caused in whole or in part by an L. intracellularis bacterium. As explained herein, L. intracellularis invades the intestinal epithelial cells and causes hyperplasia of the infected cells to lead to disease pathogenesis. Hyperplasia is an abnormal increase in the number of cells in an organ or a tissue with consequent enlargement. In animals such as pigs and horses, infection causes PE. In pigs, PE infection typically manifests in either an acute hemorrhagic form, termed “proliferative hemorrhagic enteropathy (PHE)”, usually seen in naïve adult pigs, or a more chronic wasting proliferative form termed “porcine intestinal adenomatosis (PIA)”, typically observed in growing pigs. Lawsonia infection may result in hyperplastic ileitis, typhlitis and/or colitis. In horses, L. intracellularis causes equine proliferative enteropathy (EPE), often referred to as “Lawsonia.” Infection can also be subclinical. Although symptoms of the disease are not observed, the ability to spread the pathogen through shedding in the feces remains. Thus, the term intends both clinical and subclinical disease.

The terms “polypeptide” and “protein” refer to a polymer of amino acid residues and are not limited to a minimum length of the product. Thus, peptides, oligopeptides, dimers, multimers, and the like, are included within the definition. Both full-length proteins and fragments thereof are encompassed by the definition. The terms also include postexpression modifications of the polypeptide, for example, glycosylation, acetylation, phosphorylation and the like. Furthermore, for purposes of the present invention, a “polypeptide” refers to a protein which includes modifications, such as deletions, additions and substitutions, to the native sequence, so long as the protein maintains the desired activity. These modifications may be deliberate, as through site-directed mutagenesis, or may be accidental, such as through mutations of hosts which produce the proteins or errors due to PCR amplification.

The term “peptide” as used herein refers to a fragment of a polypeptide. Thus, a peptide can include a C-terminal deletion, an N-terminal deletion and/or an internal deletion of the native polypeptide, so long as the entire protein sequence is not present. A peptide will generally include at least about 3-10 contiguous amino acid residues of the full-length molecule, and can include at least about 15-25 contiguous amino acid residues of the full-length molecule, or at least about 20-50 or more contiguous amino acid residues of the full-length molecule, or any integer between 3 amino acids and the number of amino acids in the full-length sequence, provided that the peptide in question retains the ability to elicit the desired biological response.

By “immunogenic” protein, polypeptide or peptide is meant a molecule which includes one or more epitopes and thus can modulate an immune response. Such peptides can be identified using any number of epitope mapping techniques, well known in the art. See, e.g., Epitope Mapping Protocols in Methods in Molecular Biology (2018) (Johan Rockberg and Johan Nilvebrant, Eds.) Springer, New York. For example, linear epitopes may be determined by for example, software programs, (See, e.g., Saha et al., Structure, Function, and Bioinformatics (2006) 65:40-48); or by concurrently synthesizing large numbers of peptides on solid supports, the peptides corresponding to portions of the protein molecule, and reacting the peptides with antibodies while the peptides are still attached to the supports. Similarly, conformational epitopes are readily identified by determining spatial conformation of amino acids such as by, e.g., x-ray crystallography and 2-dimensional nuclear magnetic resonance. See, e.g., Epitope Mapping Protocols, supra. Antigenic regions of proteins can also be identified using standard antigenicity and hydropathy plots, such as those calculated using, e.g., the Omiga software program available from the Oxford Molecular Group. This computer program employs the Hopp/Woods method, Hopp et al., Proc. Natl. Acad. Sci USA (1981) 78:3824-3828 for determining antigenicity profiles, and the Kyte-Doolittle technique, Kyte et al., J. Mol. Biol. (1982) 157:105-132 for hydropathy plots.

Immunogenic molecules, for purposes of the present invention, will usually be at least about 5 amino acids in length, such as at least about 10 to about 15 or more amino acids in length. There is no critical upper limit to the length of the molecule, which can comprise the full-length of the protein sequence, or even a fusion protein comprising two or more epitopes, proteins, antigens, etc.

As used herein, the term “epitope” generally refers to the site on an antigen which is recognized by a T-cell receptor and/or an antibody. Several different epitopes may be carried by a single antigenic molecule. The term “epitope” also includes modified sequences of amino acids which stimulate responses against the whole organism. The epitope can be generated from knowledge of the amino acid and corresponding DNA sequences of the polypeptide, as well as from the nature of particular amino acids (e.g., size, charge, etc.) and the codon dictionary, without undue experimentation. See, e.g., Ivan Roitt, Essential Immunology; Janis Kuby, Immunology.

An “immunological response” to an antigen or composition is the development in a subject of a humoral and/or a cellular immune response to an antigen present in the composition of interest. For purposes of the present invention, a “humoral immune response” refers to an immune response mediated by antibody molecules, while a “cellular immune response” is one mediated by T-lymphocytes and/or other white blood cells. One important aspect of cellular immunity involves an antigen-specific response by cytotoxic T-cells (“CTL”s). CTLs have specificity for peptide antigens that are presented in association with proteins encoded by the major histocompatibility complex (MHC) and expressed on the surfaces of cells. CTLs help induce and promote the destruction of intracellular microbes, or the lysis of cells infected with such microbes. Another aspect of cellular immunity involves an antigen-specific response by helper T-cells. Helper T-cells act to help stimulate the function, and focus the activity, of nonspecific, effector cells against cells displaying peptide antigens in association with MHC molecules on their surface. A “cellular immune response” also refers to the production of cytokines such as interferon γ, chemokines and other such molecules produced by activated T-cells and/or other white blood cells, including those derived from CD4+ and CD8+ T-cells.

Thus, an immunological response as used herein may be one that stimulates the production of antibodies. The antigen of interest may also elicit production of CTLs. Hence, an immunological response may include one or more of the following effects: the production of antibodies by B-cells; and/or the activation of suppressor T-cells and/or memory/effector T-cells directed specifically to an antigen or antigens present in the composition or vaccine of interest. These responses may serve to neutralize infectivity, and/or mediate antibody-complement, or antibody dependent cell cytotoxicity (ADCC) to provide protection to an immunized host. Such responses can be determined using standard immunoassays and neutralization assays, well known in the art, such as described in the Examples herein.

The innate immune system of mammals also recognizes and responds to molecular features of pathogenic organisms via activation of Toll-like receptors and similar receptor molecules on immune cells. Upon activation of the innate immune system, various non-adaptive immune response cells are activated to, e.g., produce various cytokines, lymphokines and chemokines. Cells activated by an innate immune response include immature and mature dendritic cells of the monocyte and plasmacytoid lineage (MDC, PDC), as well as gamma, delta, alpha and beta T cells and B cells and the like. Thus, the present invention also contemplates an immune response wherein the immune response involves both an innate and adaptive response.

An “immunogenic composition” is a composition that comprises an immunogenic molecule where administration of the composition to a subject results in the development in the subject of a humoral and/or a cellular immune response to the molecule of interest.

An “antigen” refers to a molecule, such as a protein, polypeptide, or fragment thereof, containing one or more epitopes (either linear, conformational or both) that will stimulate a host's immune-system to make a humoral and/or cellular antigen-specific response. The term is used interchangeably with the term “immunogen.” Antibodies such as anti-idiotype antibodies, or fragments thereof, and synthetic peptide mimotopes, which can mimic an antigen or antigenic determinant, are also captured under the definition of antigen as used herein. Similarly, an oligonucleotide or polynucleotide which expresses an antigen or antigenic determinant in vivo, such as in DNA immunization applications, is also included in the definition of antigen herein.

By “subunit vaccine” is meant a vaccine composition that includes one or more selected antigens but not all antigens, derived from or homologous to, an antigen from a pathogen of interest. Such a composition is substantially free of intact pathogen cells or pathogenic particles, or the lysate of such cells or particles. Thus, a “subunit vaccine” can be prepared from at least partially purified (preferably substantially purified) immunogenic molecules from the pathogen, or analogs thereof. The method of obtaining an antigen included in the subunit vaccine can thus include standard purification techniques, recombinant production, or synthetic production.

“Substantially purified” generally refers to isolation of a substance such that the substance comprises the majority percent of the sample in which it resides. Typically in a sample, a substantially purified component comprises at least 50%, preferably at least 80%-85%, more preferably at least 90-95%, such as at least 96%, 97%, 98%, 99%, or more of the sample. Techniques for purifying molecules of interest are well-known in the art and include, for example, ion-exchange chromatography, affinity chromatography and sedimentation according to density.

By “isolated” is meant that the indicated molecule is separate and discrete from the whole organism with which the molecule is found in nature or is present in the substantial absence of other biological macromolecules of the same type.

An “antibody” intends a molecule that “recognizes,” i.e., specifically binds to an epitope of interest present in an antigen. By “specifically binds” is meant that the antibody interacts with the epitope in a “lock and key” type of interaction to form a complex between the antigen and antibody, as opposed to non-specific binding that might occur between the antibody and, for instance, components in a mixture that includes the test substance with which the antibody is reacted. The term “antibody” as used herein includes antibodies obtained from both polyclonal and monoclonal preparations, as well as, the following: hybrid (chimeric) antibody molecules; F(ab′)₂ and F(ab) fragments; Fv molecules (non-covalent heterodimers; single-chain Fv molecules (sFv); dimeric and trimeric antibody fragment constructs; minibodies; humanized antibody molecules; and, any functional fragments obtained from such molecules, wherein such fragments retain immunological binding properties of the parent antibody molecule.

As used herein, the term “monoclonal antibody” refers to an antibody composition having a homogeneous antibody population. The term is not limited regarding the species or source of the antibody, nor is it intended to be limited by the manner in which it is made. The term encompasses whole immunoglobulins as well as fragments such as Fab, F(ab′)2, Fv, and other fragments, as well as chimeric and humanized homogeneous antibody populations, that exhibit immunological binding properties of the parent monoclonal antibody molecule.

“Homology” refers to the percent identity between two polynucleotide or two polypeptide moieties. Two nucleic acid, or two polypeptide sequences are “substantially homologous” to each other when the sequences exhibit at least about 50% sequence identity, preferably at least about 75% sequence identity, more preferably at least about 80%-85% sequence identity, more preferably at least about 90% sequence identity, and most preferably at least about 95%-98% sequence identity over a defined length of the molecules. As used herein, substantially homologous also refers to sequences showing complete identity to the specified sequence.

In general, “identity” refers to an exact nucleotide-to-nucleotide or amino acid-to-amino acid correspondence of two polynucleotides or polypeptide sequences, respectively. Percent identity can be determined by a direct comparison of the sequence information between two molecules by aligning the sequences, counting the exact number of matches between the two aligned sequences, dividing by the length of the shorter sequence, and multiplying the result by 100. Readily available computer programs can be used to aid in the analysis. See, e.g., molbiol-tools.ca/alignments for a list of computer programs to determine similarity between two or more amino acid or nucleotide sequences. These programs are readily utilized with the default parameters recommended by the manufacturer. For example, percent identity of a particular nucleotide sequence to a reference sequence can be determined using the homology Smith-Waterman algorithm with a default scoring table and a gap penalty of six nucleotide positions.

Another method of establishing percent identity in the context of the present invention is to use the MPSRCH package of programs copyrighted by the University of Edinburgh, developed by John F. Collins and Shane S. Sturrok, and distributed by IntelliGenetics, Inc. (Mountain View, CA). From this suite of packages the Smith-Waterman algorithm can be employed where default parameters are used for the scoring table (for example, gap open penalty of 12, gap extension penalty of one, and a gap of six). From the data generated the “Match” value reflects “sequence identity.” Other suitable programs for calculating the percent identity or similarity between sequences are generally known in the art, for example, another alignment program is BLAST, used with default parameters. For example, BLASTN and BLASTP can be used using the following default parameters: genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+Swiss protein+Spupdate+PIR. Details of these programs are readily available.

Alternatively, homology can be determined by hybridization of polynucleotides under conditions which form stable duplexes between homologous regions, followed by digestion with single-stranded-specific nuclease(s), and size determination of the digested fragments. DNA sequences that are substantially homologous can be identified in a Southern hybridization experiment under, for example, stringent conditions, as defined for that particular system. Defining appropriate hybridization conditions is within the skill of the art. See, e.g., Sambrook et al., Molecular Cloning, a laboratory manual, Cold Spring Harbor Laboratories, New York.

The terms “polynucleotide,” “oligonucleotide,” “nucleic acid” and “nucleic acid molecule” are used herein to include a polymeric form of nucleotides of any length, either ribonucleotides or deoxyribonucleotides. This term refers only to the primary structure of the molecule. Thus, the term includes triple-, double- and single-stranded DNA, as well as triple-, double- and single-stranded RNA. It also includes modifications, such as by methylation and/or by capping, and unmodified forms of the polynucleotide. More particularly, the terms “polynucleotide,” “oligonucleotide,” “nucleic acid” and “nucleic acid molecule” include polydeoxyribonucleotides (containing 2-deoxy-D-ribose), polyribonucleotides (containing D-ribose), any other type of polynucleotide which is an N- or C-glycoside of a purine or pyrimidine base, and other polymers containing nonnucleotidic backbones, for example, polyamide (e.g., peptide nucleic acids (PNAs)) and polymorpholino (commercially available from the Anti-Virals, Inc., Corvallis, Oregon, as Neugene) polymers, and other synthetic sequence-specific nucleic acid polymers providing that the polymers contain nucleobases in a configuration which allows for base pairing and base stacking, such as is found in DNA and RNA. There is no intended distinction in length between the terms “polynucleotide,” “oligonucleotide,” “nucleic acid” and “nucleic acid molecule,” and these terms will be used interchangeably. Thus, these terms include, for example, 3′-deoxy-2′,5′-DNA, oligodeoxyribonucleotide N3′ P5′ phosphoramidates, 2′-O-alkyl-substituted RNA, double- and single-stranded DNA, as well as double- and single-stranded RNA, DNA:RNA hybrids, and hybrids between PNAs and DNA or RNA, and also include known types of modifications, for example, labels which are known in the art, methylation, “caps,” substitution of one or more of the naturally occurring nucleotides with an analog, internucleotide modifications such as, for example, those with uncharged linkages (e.g., methyl phosphonates, phosphotriesters, phosphoramidates, carbamates, etc.), with negatively charged linkages (e.g., phosphorothioates, phosphorodithioates, etc.), and with positively charged linkages (e.g., aminoalkylphosphoramidates, aminoalkylphosphotriesters), those containing pendant moieties, such as, for example, proteins (including nucleases, toxins, antibodies, signal peptides, poly-L-lysine, etc.), those with intercalators (e.g., acridine, psoralen, etc.), those containing chelators (e.g., metals, radioactive metals, boron, oxidative metals, etc.), those containing alkylators, those with modified linkages (e.g., alpha anomeric nucleic acids, etc.), as well as unmodified forms of the polynucleotide or oligonucleotide. In particular, DNA is deoxyribonucleic acid.

“Recombinant” as used herein to describe a nucleic acid molecule means a polynucleotide of genomic, cDNA, viral, semisynthetic, or synthetic origin which, by virtue of its origin or manipulation is not associated with all or a portion of the polynucleotide with which it is associated in nature. The term “recombinant” as used with respect to a protein or polypeptide means a polypeptide produced by expression of a recombinant polynucleotide. In general, the gene of interest is cloned and then expressed in transformed organisms, as described further below. The host organism expresses the foreign gene to produce the protein under expression conditions.

“Recombinant host cells”, “host cells,” “cells”, “cell lines,” “cell cultures”, and other such terms denoting microorganisms or higher eukaryotic cell lines cultured as unicellular entities refer to cells which can be, or have been, used as recipients for recombinant vector or other transferred DNA, and include the original progeny of the original cell which has been transfected.

A “coding sequence” or a sequence which “encodes” a selected polypeptide, is a nucleic acid molecule which is transcribed (in the case of DNA) and translated (in the case of mRNA) into a polypeptide in vitro or in vivo when placed under the control of appropriate regulatory sequences (or “control elements”). The boundaries of the coding sequence can be determined by a start codon at the 5′ (amino) terminus and a translation stop codon at the 3′ (carboxy) terminus. A coding sequence can include, but is not limited to, cDNA from viral, procaryotic or eucaryotic mRNA, genomic DNA sequences from viral or procaryotic DNA, and even synthetic DNA sequences. A transcription termination sequence may be located 3′ to the coding sequence.

Typical “control elements,” include, but are not limited to, transcription promoters, transcription enhancer elements, transcription termination signals, polyadenylation sequences (located 3′ to the translation stop codon), sequences for optimization of initiation of translation (located 5′ to the coding sequence), and translation termination sequences. “Operably linked” refers to an arrangement of elements wherein the components so described are configured so as to perform their usual function. Thus, a given promoter operably linked to a coding sequence is capable of effecting the expression of the coding sequence when the proper enzymes are present. The promoter need not be contiguous with the coding sequence, so long as it functions to direct the expression thereof. Thus, for example, intervening untranslated yet transcribed sequences can be present between the promoter sequence and the coding sequence and the promoter sequence can still be considered “operably linked” to the coding sequence.

“Expression cassette” or “expression construct” refers to an assembly which is capable of directing the expression of the sequence(s) or gene(s) of interest. An expression cassette generally includes control elements, as described above, such as a promoter which is operably linked to (so as to direct transcription of) the sequence(s) or gene(s) of interest, and often includes a polyadenylation sequence as well. Within certain embodiments of the invention, the expression cassette described herein may be contained within a plasmid construct. In addition to the components of the expression cassette, the plasmid construct may also include, one or more selectable markers, a signal which allows the plasmid construct to exist as single-stranded DNA (e.g., a M13 origin of replication), at least one multiple cloning site, and a “mammalian” origin of replication (e.g., a SV40 or adenovirus origin of replication).

The term “transform” is used to refer to the uptake of foreign DNA by a cell. A cell has been “transformed” when exogenous DNA has been introduced inside the cell membrane. A number of transformation techniques are generally known in the art. See, e.g., Sambrook et al., Molecular Cloning, a laboratory manual, Cold Spring Harbor Laboratories, New York; Davis et al. Basic Methods in Molecular Biology, Elsevier. Such techniques can be used to introduce one or more exogenous DNA moieties into suitable host cells. The term refers to both stable and transient uptake of the genetic material, and includes uptake of peptide- or antibody-linked DNAs.

A “vector” is capable of transferring nucleic acid sequences to target cells (e.g., viral vectors, non-viral vectors, particulate carriers, and liposomes). Typically, “vector construct,” “expression vector,” and “gene transfer vector,” mean any nucleic acid construct capable of directing the expression of a nucleic acid of interest and which can transfer nucleic acid sequences to target cells. Thus, the term includes cloning and expression vehicles, as well as viral vectors.

“Gene transfer” or “gene delivery” refers to methods or systems for reliably inserting DNA or RNA of interest into a host cell. Such methods can result in transient expression of non-integrated transferred DNA, extrachromosomal replication and expression of transferred replicons (e.g., episomes), or integration of transferred genetic material into the genomic DNA of host cells. Gene delivery expression vectors include, but are not limited to, vectors derived from bacterial plasmid vectors, viral vectors, non-viral vectors, alphaviruses, pox viruses and vaccinia viruses. When used for immunization, such gene delivery expression vectors may be referred to as vaccines or vaccine vectors.

By “vertebrate subject” is meant any member of the subphylum chordata, including, without limitation, humans and other primates, including non-human primates such as chimpanzees and other apes and monkey species; farm animals such as cattle, sheep, pigs, goats and horses; domestic mammals such as dogs and cats; non-domestic animals such as elk, deer, mink and feral cats; laboratory animals including rodents such as mice, rats and guinea pigs; birds, including domestic, wild and game birds such as chickens, turkeys and other gallinaceous birds, ducks, geese, pheasant, emu, ostrich and the like. The term does not denote a particular age. Thus, both adult and newborn individuals are intended to be covered.

By “therapeutically effective amount” in the context of the immunogenic compositions described herein is meant an amount of an immunogen which will induce an immunological response as described herein, either for antibody production or for treatment or prevention of infection.

As used herein, “treatment” refers to, without limitation, any of (i) the prevention of infection or reinfection, as in a traditional vaccine, and/or (ii) the reduction or elimination of symptoms from an infected individual. Treatment may be effected prophylactically (prior to infection) or therapeutically (following infection). Additionally, prevention or treatment in the context of the present invention can be prevention or reduction of the amount of bacteria present in the treated subject, or in feces of the treated subject.

### 2. MODES OF CARRYING OUT THE INVENTION

Before describing the present invention in detail, it is to be understood that this invention is not limited to particular formulations or process parameters as such may, of course, vary. It is also to be understood that the terminology used herein is for the purpose of describing particular embodiments of the invention only, and is not intended to be limiting.

Although a number of methods and materials similar or equivalent to those described herein can be used in the practice of the present invention, the preferred materials and methods are described herein.

The present invention is based in part on the discovery of immunogenic L. intracellularis molecules using a unique combination of two-dimensional electrophoresis (2DE), Western blotting (WB) and mass spectrometry (MS). These molecules include one or more epitopes for stimulating an immune response in a subject of interest. The molecules can be provided in an isolated form as discrete components, or as fusion proteins. Antigens can be incorporated into pharmaceutical compositions, such as vaccine compositions. As shown in the examples, serum from animals immunized with subunit vaccine compositions including L. intracellularis recombinant proteins showed an inhibitory effect on the attachment and penetration of live, avirulent L. intracellularis, thus indicating these proteins were able to produce neutralizing antibodies in order to prevent L. intracellularis infection.

The present invention thus provides immunological compositions and methods for treating and/or preventing L. intracellularis disease. Immunization can be achieved by any of the methods known in the art including, but not limited to, use of vaccines containing isolated L. intracellularis antigens or fusion proteins comprising multiple antigens, or by passive immunization using antibodies directed against the antigens. Such methods are described in detail below. Moreover, the antigens described herein can be used for detecting the presence of L. intracellularis bacteria, for example in a biological sample.

The vaccines are useful in vertebrate subjects that are susceptible to L. intracellularis infection, including without limitation, mammalian and avian species such as, but not limited to, pigs, horses, primates, dogs, rats, guinea pigs, rabbits and hamsters.

In order to further an understanding of the invention, a more detailed discussion is provided below regarding L. intracellularis antigens, production thereof, compositions comprising the same, and methods of using such compositions in the treatment and/or prevention of infection, as well as in the diagnosis of infection.

A. L. intracellularis Antigens

Antigens for use in the subject compositions can be derived from any of the several L. intracellularis strains and isolates. As explained herein, L. intracellularis has the capability to infect a number of vertebrates, including mammalian and avian species. Infection can cause proliferative enteropathy (PE) and have a profound economic impact on the animal industry.

Table 1, Table 2 and FIGS. 1B, 2B, 3B, 4B, 5B, 6B, 7B, 8B, 9B, 10B, and 11B (SEQ ID NOs: 2, 5, 8, 11, 14, 17, 20, 23, 26, 29 and 32, respectively), show representative antigens for use in compositions for stimulating immune responses against L. intracellularis. Molecules listed in Tables 1 and 2 and in the figures were identified as immunogenic and, those proteins detailed in SEQ ID NOs 2, 5, 8, 11 and 29 as described in the Examples, reacted with hyperimmune serum from pigs infected with pathogenic L. intracellularis.

Preferably, the subject compositions include one or more of these antigens, such as one, two, three, four, five, six, seven, eight, nine, ten, or more of the antigens in any combination, as well as antigens from other L. intracellularis strains or isolates that correspond to the L. intracellularis antigens listed in the tables and shown in the figures. Moreover, the antigens present in the compositions can include the full-length amino acid sequences, or fragments or variants of these sequences, so long as the antigens stimulate an immunological response, preferably, a neutralizing and/or protective immune response. Thus, the antigens can be provided with deletions from the N- or C-termini which do not disrupt immunogenicity, including without limitation, deletions of an N-terminal methionine if present, deletions of all or part of the transmembrane domain(s) if present, deletions of all or part of the cytoplasmic domain(s) if present, and deletions of the native signal sequence if present. Additionally, the molecules can include other N-terminal, C-terminal and internal deletions of amino acids or sequences irrelevant to immunogenicity. Moreover, the molecules can include additions, such as the presence of a heterologous signal sequence if desired, as well as amino acid linkers, and/or ligands useful in protein purification, such as histidine tags, glutathione-S-transferase or staphylococcal protein A.

Representative proteins are described in detail below and are shown in Table 1. It is to be understood that the present invention is not limited to the use of these representative proteins as a number of strains and isolates of L. intracellularis are known, and the corresponding proteins from these strains and isolates are intended to be captured herein.

TABLE 1
Representative L. intracellularis proteins

| SEQ ID | | | Uniprot | | |
|---|---|---|---|---|---|
| NOs. | Gene | NCBI No. | No. | Genomic position | Function |
| 1, 2 | LI0710 | CAJ54764 | Q1MQG3 | 894199 . . . 895080 | Flagellin |
| 4, 5 | LI0649 | CAJ54703 | Q1MQM4 | 802639 . . . 805194 | autotransporter |
| 7, 8 | LI0169 | CAJ54225 | Q1MS01 | 211599 . . . 213256 | ABC dipeptide |
| | | | | | transport system |
| 10, 11 | LI1153 | CAJ55207 | Q1MP70 | 1413348 . . . 1414544 | Putative protein N |
| 13, 14 | LI0786 | CAJ54840 | Q1MQ87 | complement | DNA polymerase III |
| | | | | 978042 . . . 979193 | subunit beta |
| 16, 17 | LI1171 | CAJ55225 | Q1MP52 | complement | 5′nucleotidase/2′3′ |
| | | | | 1432770 . . . 1434458 | cyclic |
| | | | | | phosphodiesterase |
| 19, 20 | LI0608 | CAJ54662 | Q1MQR5 | complement | Cysteine-tRNA ligase |
| | | | | 751382 . . . 752839 | |
| 22, 23 | LI0726 | CAJ54780 | Q1MQE7 | complement | S-adenosylmethionine |
| | | | | 911020 . . . 912234 | synthase |
| 25, 26 | LI0823 | CAJ54877 | Q1MQ50 | 1022176 . . . 1023267 | Xaa-Pro |
| | | | | | aminopeptidase |
| 28, 29 | LI0625 | CAJ54679 | Q1MQP8 | 770925 . . . 772571 | 60 kDa chaperonin, |
| | | | | | groL, GroEL |
| 31, 32 | LI0794 | CAJ54848 | Q1MQ79 | 983737 . . . 989366 | ATP-dependant Clp |
| | | | | | protease proteolytic |
| | | | | | subunit |

LI0710, also termed flagellin, flag and filC herein, is found on the outside of the outer membrane cell surface of L. intracellularis. Flagellin is the subunit protein that polymerizes to form the filaments of bacterial flagella and plays an important role in bacterial locomotion and chemotaxis. As described herein, LI0710 is immunogenic, has both adjuvant and antigenic properties, and induces a specific immune response in intestinal mucosa of animals. Flagellin is a TLR5 agonist and plays an important role in the process of immune recognition of Gram negative bacteria. Due to its dual antigen and adjuvant nature, L. intracellularis rLI0710 is ideal for use in a subunit vaccine.

A representative native DNA sequence (SEQ ID NO:1) and corresponding amino acid sequence (SEQ ID NO: 2) of LI0710 are shown in FIGS. 1A and 1B, respectively. The native protein shown (SEQ ID NO: 2) in FIG. 1B includes 293 amino acids and consists of a PF00669 domain between amino acids 5-141 and a PF00700 domain between amino acids 208-291, as predicted using the bioinformatics tool PFAM, a database of protein families (pfam.xfam.org).

The recombinant protein produced in the examples included an N-terminal sequence comprising a His-tag and linker for purification purposes (MHHHHHHGS) (SEQ ID NO: 3, FIG. 1C) and the N-terminal methionine of the native molecule was excluded from the recombinant protein. Thus, a representative recombinant molecule for use in the immunogenic compositions, with the removal of the His-tag-containing sequence, includes amino acids 2-293 of the native molecule shown in FIG. 1B (SEQ ID NO: 2).

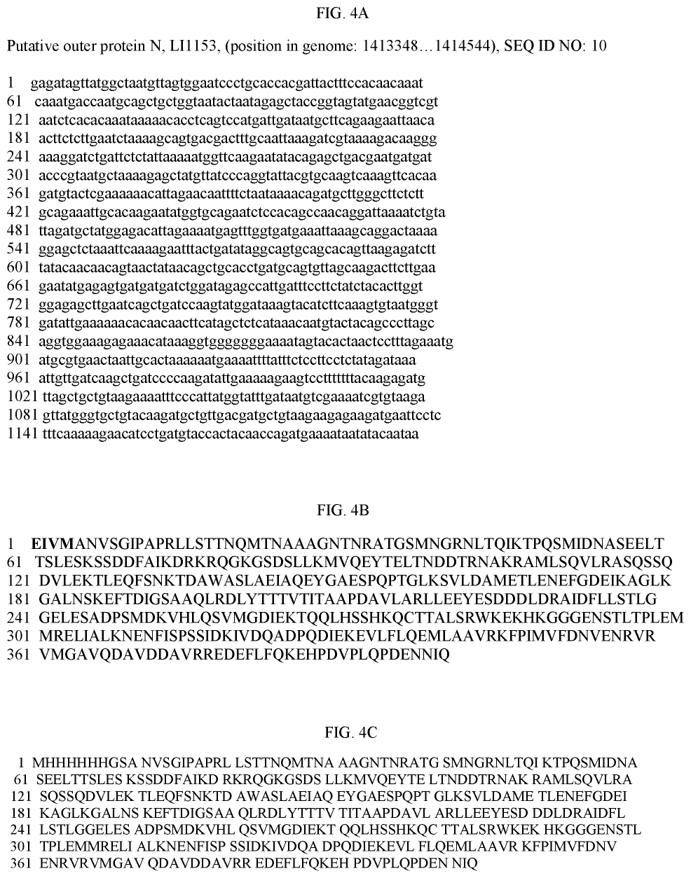

LI1153 is found on the outer membrane cell surface of L. intracellularis and is annotated as putative outer protein N. As shown herein, this molecule is also immunogenic. A representative native DNA sequence (SEQ ID NO: 10) and corresponding amino acid sequence (SEQ ID NO: 11) of LI1153 are shown in FIGS. 4A and 4B, respectively. The native protein (SEQ ID NO: 11) shown in FIG. 4B includes 398 amino acids. LI1153 is part of the T3 SS system and consists of two prominent domains with important functions during invasion into eukaryotic cells: HrpJ, positioned between amino acids 63-222 of SEQ ID NO: 11 (PF07201 (PFAM)), and TyeA, positioned between amino acids 299-378 of SEQ ID NO: 11 (PF09059 (PFAM)). The HrpJ domain is predicted to be part of the T3SS. Based on a comparison of the structure of other T3 SS systems, it appears that LI1153 corresponds to the predicted second part of the L. intracellularis T3SS and has a role in controlling secretion of effector proteins into host cells. Given the interaction between this protein and target cells as described herein, the protein likely plays a role in invasion and attachment of the bacteria to small intestine enterocytes.

The protein produced in the examples has included an N-terminal sequence comprising the His-tag and linker (MHHHHHHGS) as described above (SEQ ID NO: 12, FIG. 4C) as well as it deleted the first 4 amino acids in the native molecule shown in FIG. 4B. Thus, a representative recombinant molecule, for use in the immunogenic compositions, with the removal of the His-tag-containing sequence, as well as the 4 N-terminal amino acids of the native molecule, includes amino acids 5-398 of the native molecule (SEQ ID NO: 11) shown in FIG. 4B.

LI0169 is a transmembrane protein and appears to be expressed on the bacterial membrane as part of the ATP-binding cassette (ABC) transporter complex. In bacteria, the ABC transporter complex plays a central role in the uptake of sugars, amino acids, metals, growth factors, ions and other solutes across the cell membrane. As shown herein, this protein is also immunogenic.

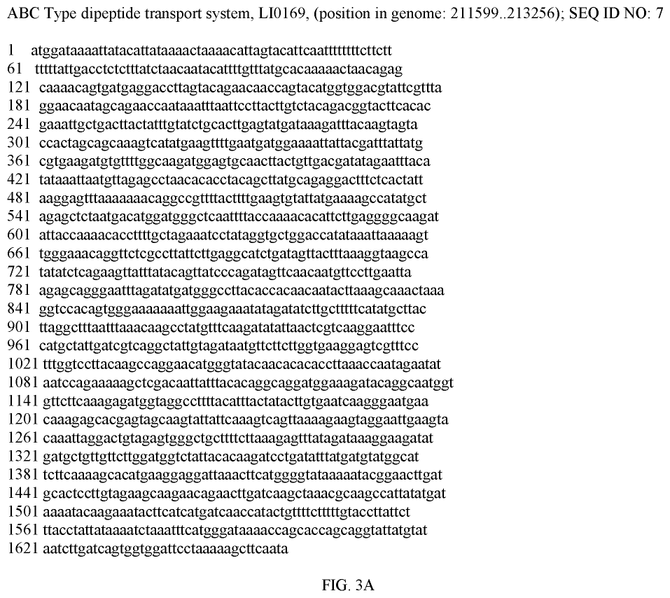

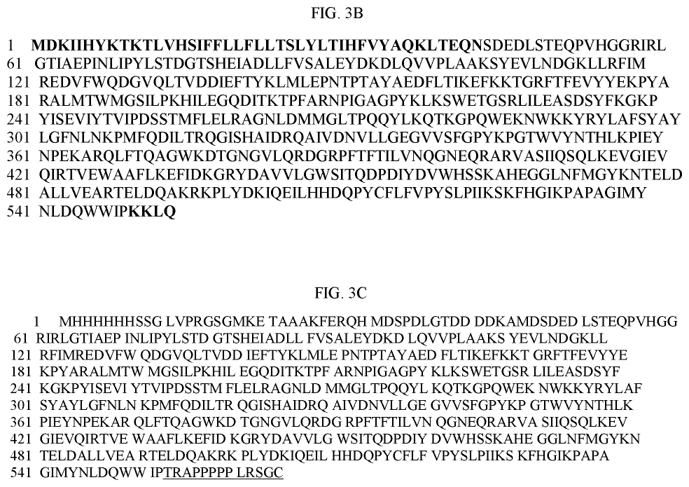

A representative native DNA sequence (SEQ ID NO: 7) and corresponding amino acid sequence (SEQ ID NO: 8) of LI0169 are shown in FIGS. 3A and 3B, respectively. The native protein shown in SEQ ID NO: 8 includes 552 amino acids and includes a transmembrane helical domain, (amino acids 12-34), a periplasmic domain (amino acids 98-456, PF00496) and an ATP-binding coiled domain (amino acids 476-496) at the intracellular face of the membrane that together form a central pore. It transports di- and tripeptides in an ATP-dependent manner.

Amino acids 1-42 for the native protein (SEQ ID NO: 8, FIG. 4B) were removed during cloning (bolded amino acids in SEQ ID NO: 9, FIG. 3C). The recombinant protein produced in the examples has a His-tag and linker (MHHHHHHSSG LVPRGSGMKE TAAAKFERQH MDSPDLGTDD DDKAMD, amino acids 1-46 of SEQ ID NO: 9). Additionally, the recombinant molecule had a deletion of the last 4 C-terminal amino acids of the native molecule shown in FIG. 4B (SEQ ID NO: 8, bolded amino acids). The molecule includes an additional 13 amino acids at the C-terminus. Thus, the recombinant protein (SEQ ID NO: 9) includes the His-Tag, it excludes the transmembrane domain amino acids as well as the last 4 C-terminal amino acids in the native sequence (all together amino acids 43-552) and it includes an additional 13 amino acids in the C-terminus.

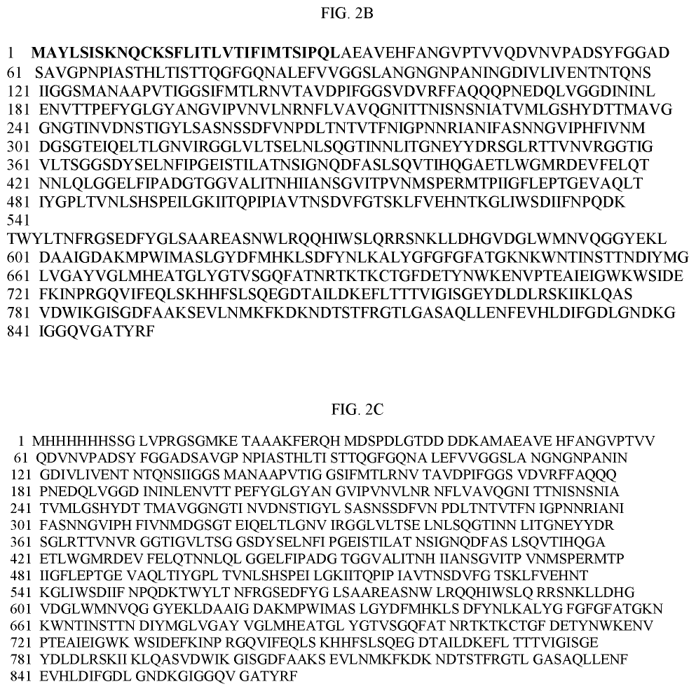

LI0649, also known as LatA, is an autotransporter and as shown herein, is immunogenic. LI0649 plays a role in bacterial-host interactions. This molecule is a transmembrane protein. A representative native DNA sequence (SEQ ID NO: 4) and corresponding amino acid sequence (SEQ ID NO: 5) of LI0649 is shown in FIGS. 2A and 2B, respectively. The native protein shown in SEQ ID NO: 5 includes 851 amino acids and excludes the 30 amino acids in the N-terminal transmembrane domain (bolded amino acids).

The recombinant protein (SEQ ID NO: 6, FIG. 2C) produced in the examples included an N-terminal sequence comprising a His-tag and linker for purification purposes (MHHHHHHSSG LVPRGSGMKE TAAAKFERQH MDSPDLGTDD DDKAMD), as described above. Additionally, amino acids 1-30, which included the N-terminal transmembrane domain, were deleted from the native protein shown in SEQ ID NO: 5. Thus, a representative recombinant molecule for use in the immunogenic compositions, with the removal of the His-tag-containing sequence as well as the N-terminal amino acids in the native molecule, includes amino acids 31-851 of the native molecule shown in FIG. 2B.

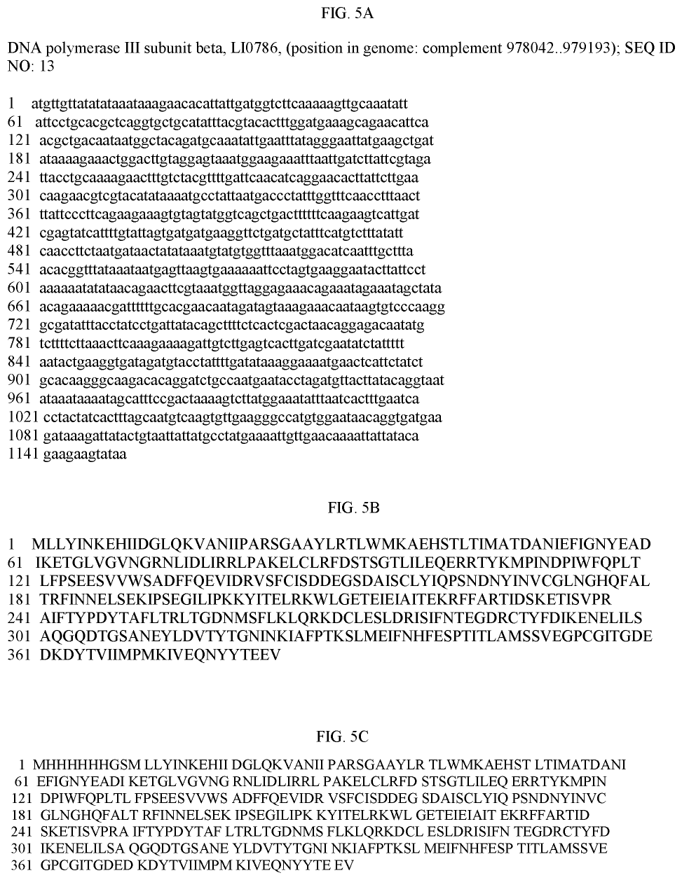

LI0786 is a DNA polymerase III subunit B molecule with catalytic activity. A representative native DNA sequence (SEQ ID NO: 13) and corresponding amino acid sequence (SEQ ID NO: 14) of LI0786 is shown in FIGS. 5A and 5B, respectively. The native protein shown in SEQ ID NO: 14 includes 383 amino acids. The recombinant protein with the His-tag and linker (MHHHHHHGS) is shown in SEQ ID NO: 15 (FIG. 5C).

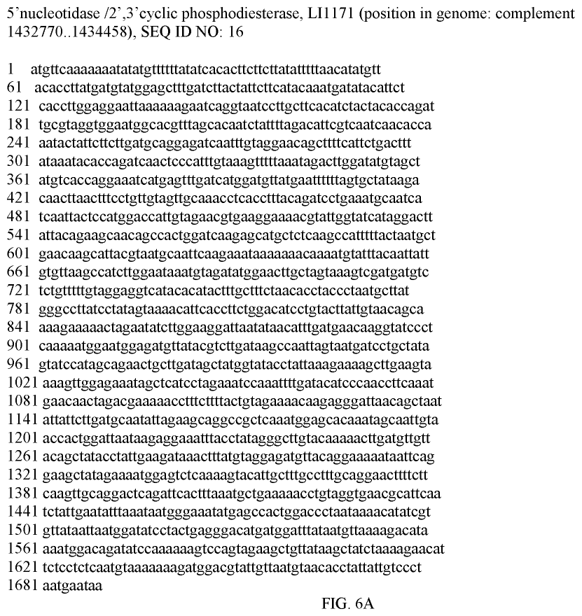

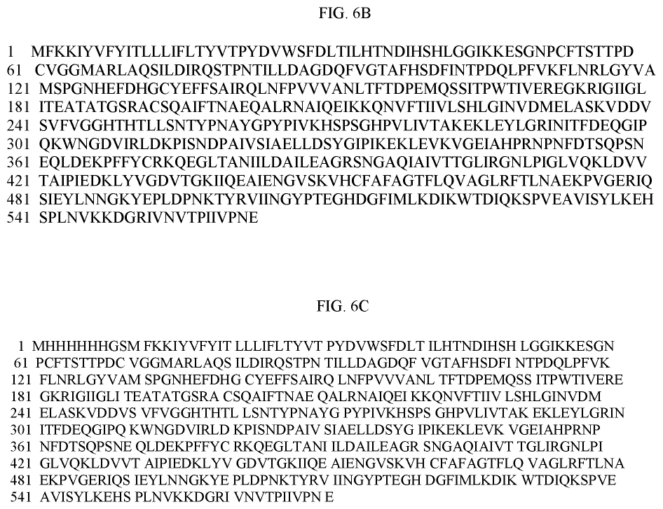

LI1171 is a 5′-nucleotidase/2′,3′-cyclic phosphodiesterase and displays hydrolase activity, acting on ester bonds, metal ion binding, and nucleotide binding. A representative native DNA sequence (SEQ ID NO: 16) and corresponding amino acid sequence (SEQ ID NO: 17) of LI1171 is shown in FIGS. 6A and 6B, respectively. The native protein shown in SEQ ID NO: 17 includes 562 amino acids. The recombinant protein with the His-tag and linker (MHHHHHHGS) is shown in SEQ ID NO: 18 (FIG. 6C).

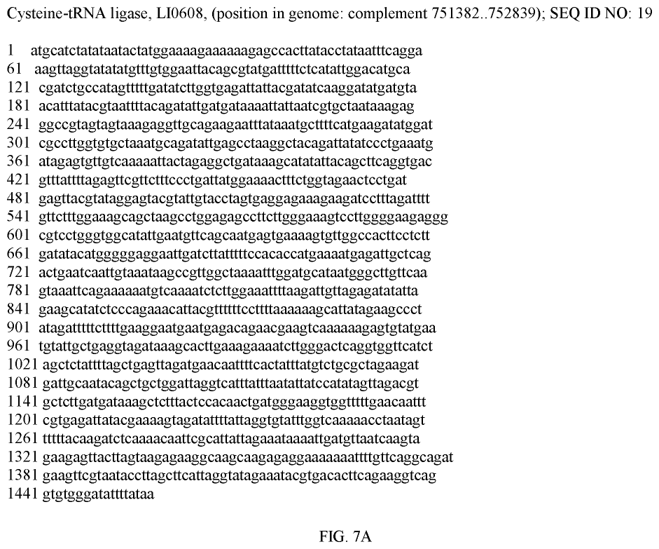

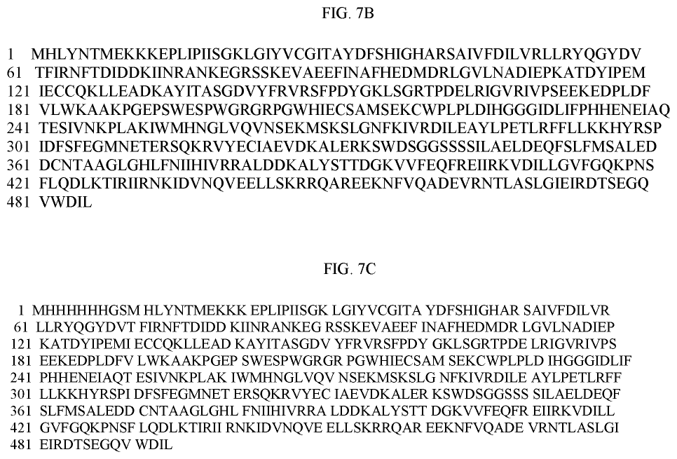

LI0608 is a cysteine-tRNA ligase. A representative native DNA sequence (SEQ ID NO: 19) and corresponding amino acid sequence (SEQ ID NO: 20) of LI0608 is shown in FIGS. 7A and 7B, respectively. The native protein shown in SEQ ID NO: 20 includes 485 amino acids. The recombinant protein with the His-tag and linker (MHHHHHHGS) is shown in SEQ ID NO: 21 (FIG. 7C).

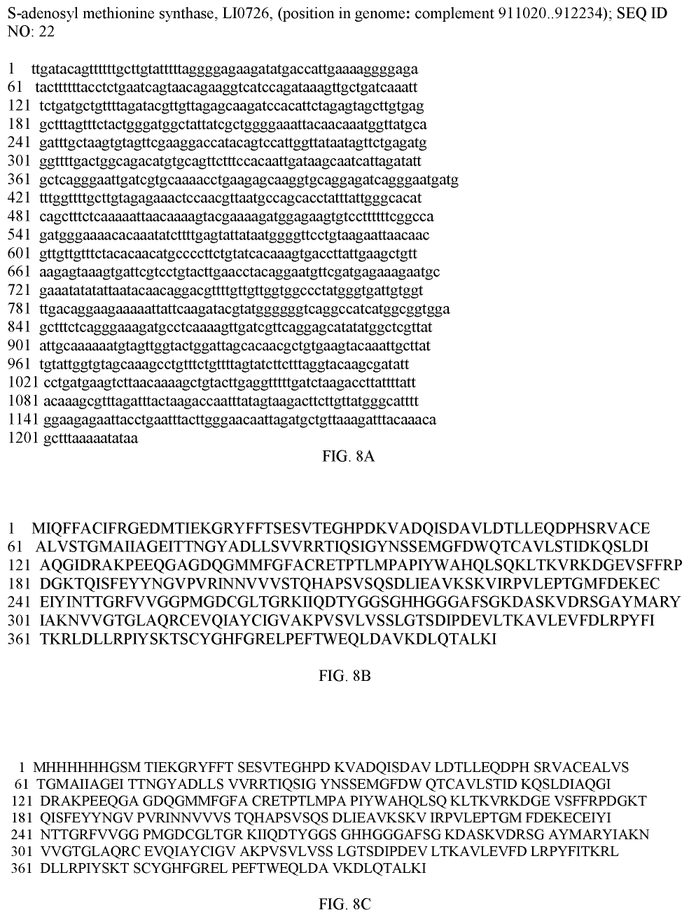

LI0726 is a S-adenosylmethionine synthase and is located in the cytoplasm of the cell. It catalyzes the formation of S-adenosylmethionine (AdoMet) from methionine and ATP. A representative native DNA sequence (SEQ ID NO: 22) and corresponding amino acid sequence (SEQ ID NO: 23) of LI0726 is shown in FIGS. 8A and 8B, respectively. The native protein shown in SEQ ID NO: 23 includes 404 amino acids. The recombinant protein with the His-tag and linker (MHHHHHHGS) is shown in SEQ ID NO: 24 (FIG. 8C).

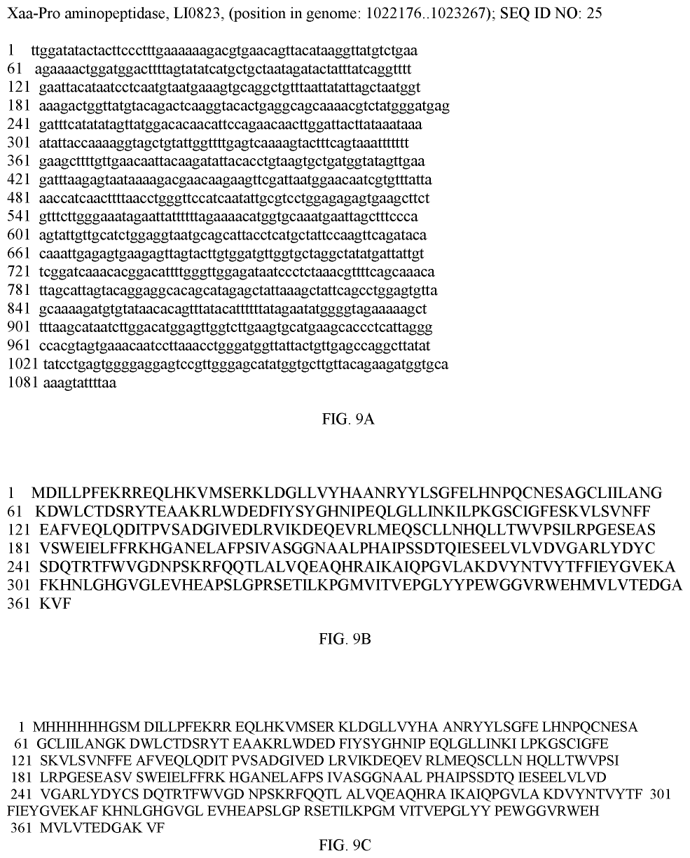

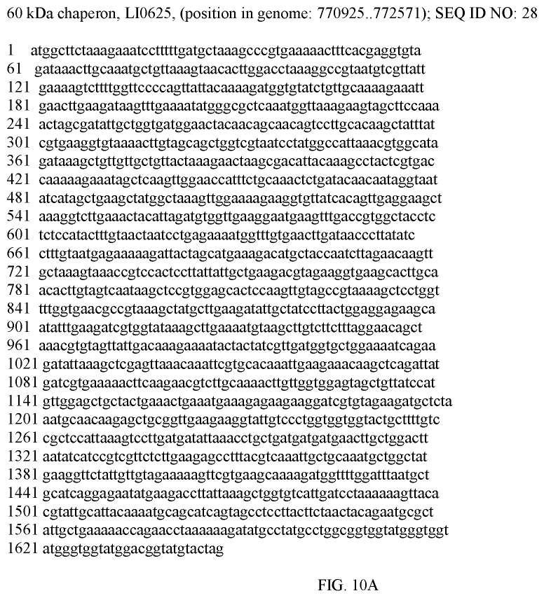

LI0823 is a Xaa-Pro-aminopeptidase that catalyses the hydrolysis of N-terminal amino acid residues in a polypeptide chain and has aminopeptidase activity. A representative native DNA sequence (SEQ ID NO: 25) and corresponding amino acid sequence (SEQ ID NO: 26) of LI0823 is shown in FIGS. 9A and 9B, respectively. The native protein shown in SEQ ID NO: 26 includes 363 amino acids. The recombinant protein with the His-tag and linker (MHHHHHHGS) is shown in SEQ ID NO: 27 (FIG. 9C).

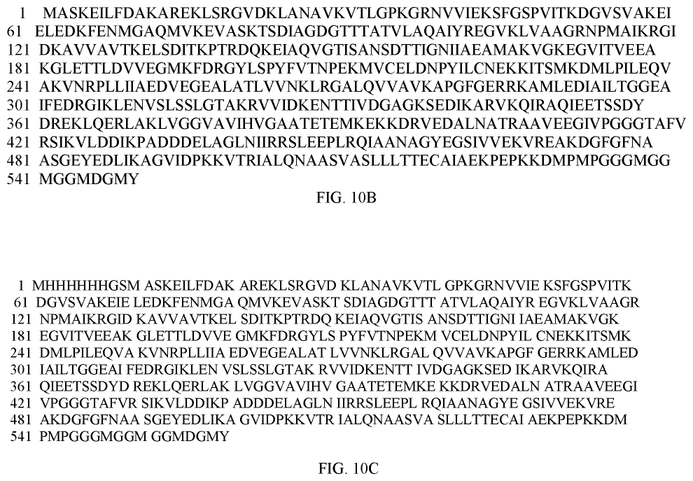

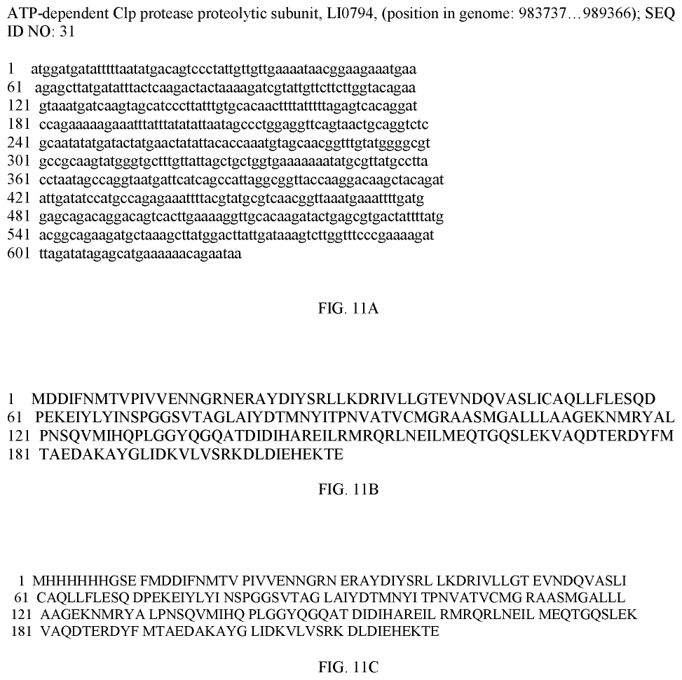

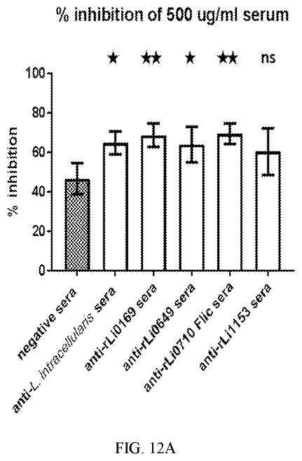

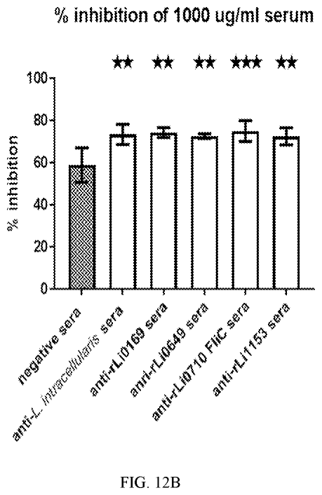

LI0625 is a 60 kDa chaperonin that prevents misfolding and promotes the refolding and proper assembly of unfolded polypeptides generated under stress conditions. A representative native DNA sequence (SEQ ID NO: 28) and corresponding amino acid sequence (SEQ ID NO: 29) of LI0625 is shown in FIGS. 10A and 10B, respectively. The native protein shown in SEQ ID NO: 29 includes 548 amino acids. The recombinant protein with the His-tag and linker (MHHHHHHGS) is shown in SEQ ID NO: 30 (FIG. 10C).

LI0794 is an ATP-dependent Clp protease proteolytic subunit and is found intracellularly. It cleaves peptides in various proteins in a process that requires ATP hydrolysis, has a chymotrypsin-like activity, and plays a major role in the degradation of misfolded proteins. A representative native DNA sequence (SEQ ID NO: 31) and corresponding amino acid sequence (SEQ ID NO: 32) of LI0794 is shown in FIGS. 11A and 11B, respectively. The native protein shown in SEQ ID NO: 32 includes 209 amino acids. The recombinant protein with the His-tag and linker (MHHHHHHGSEF) is shown in SEQ ID NO: 33 (FIG. 11C).

As explained above, any of these L. intracellularis antigens, as well as the corresponding antigens from different strains and isolates, can be used alone or in combination in the immunogenic compositions described herein, to provide protection against L. intracellularis infection. The compositions can include L. intracellularis antigens from more than one strain or isolate. Thus, each of the components of a subunit composition or fusion protein can be obtained from the same L. intracellularis strain or isolate, or from different L. intracellularis strains or isolates.

Moreover, the L. intracellularis antigens present in subunit compositions can include various combinations of any of the L. intracellularis proteins described herein, such as one or more of rLI0710, rLI1153, rLI0169, LI0649, rLI0786, rLI1171, rLI0608, rLI0726, rLI0823, rLI0625, and rLI0794.

The immunogenic compositions can include discrete antigens, i.e., isolated and purified antigens provided separately, or can include fusions of the desired antigens. The fusions will include two or more immunogenic L. intracellularis proteins, such as two, three, four, five, six, seven, eight, nine, ten, etc., such as one or more of the L. intracellularis antigens described herein, or antigens from other L. intracellularis strains or isolates that correspond to the L. intracellularis antigens. Moreover, as explained above, the antigens present in the fusions can include the full-length amino acid sequences, or fragments or variants of these sequences so long as the antigens stimulate an immunological response, preferably, a protective immune response. At least one epitope from these antigens will be present. In some embodiments, the fusions will include repeats of desired epitopes. As explained above, the antigens present in fusions can be derived from the same L. intracellularis strain or isolate, or from different strains or isolates, to provide increased protection against a broad range of L. intracellularis bacteria.

In certain embodiments, the fusions include multiple antigens, such as more than one epitope from a particular L. intracellularis antigen, and/or epitopes from more than one L. intracellularis antigen. The epitopes can be provided as the full-length antigen sequence, or in a partial sequence that includes the epitope. The epitopes can be from the same L. intracellularis strain or isolate, or different L. intracellularis strains or isolates. Additionally, the epitopes can be derived from the same L. intracellularis protein or from different L. intracellularis proteins from the same or different L. intracellularis strain or isolate.

More particularly, chimeric fusion proteins may comprise multiple epitopes, a number of different L. intracellularis proteins from the same or different strains or isolates, as well as multiple or tandem repeats of selected L. intracellularis sequences, multiple or tandem repeats of selected L. intracellularis epitopes, or any conceivable combination thereof. Epitopes may be identified using techniques as described herein, or fragments of L. intracellularis proteins may be tested for immunogenicity and active fragments used in compositions in lieu of the entire polypeptide. Fusions may also include the full-length sequence.

The antigen sequences present in the fusions may be separated by spacers, but need not be. A selected spacer sequence may encode a wide variety of moieties of one or more amino acids in length. Selected spacer groups may also provide enzyme cleavage sites so that the expressed chimeric molecule can be processed by proteolytic enzymes in vivo to yield a number of peptides.

For example, amino acids can be used as spacer sequences. Such spacers will typically include from 1-500 amino acids, such as 1-100 amino acids, e.g., 1-50 amino acids, such as 1-25 amino acids, 1-10 amino acids, 1-5 amino acids, or any integer between 1-500. The spacer amino acids may be the same or different between the various antigens. Particularly preferred amino acids for use as spacers are amino acids with small side groups, such as serine, alanine, glycine and valine. Various combinations of amino acids or repeats of the same amino acid may be used.

In order to enhance immunogenicity of the L. intracellularis proteins, as well as multiple antigen fusion molecules, they may be conjugated with a carrier. By “carrier” is meant any molecule which when associated with an antigen of interest, imparts immunogenicity to the antigen. Examples of suitable carriers include large, slowly metabolized macromolecules such as: proteins; polysaccharides, such as sepharose, agarose, cellulose, cellulose beads and the like; polymeric amino acids such as polyglutamic acid, polylysine, and the like; amino acid copolymers; inactive virus particles; bacterial toxins such as tetanus toxoid, serum albumins, keyhole limpet hemocyanin, thyroglobulin, ovalbumin, sperm whale myoglobin, and other proteins well known to those skilled in the art.

These carriers may be used in their native form or their functional group content may be modified by, for example, succinylation of lysine residues or reaction with Cys-thiolactone. A sulfhydryl group may also be incorporated into the carrier (or antigen) by, for example, reaction of amino functions with 2-iminothiolane or the N-hydroxysuccinimide ester of 3-(4-dithiopyridyl) propionate. Suitable carriers may also be modified to incorporate spacer arms (such as hexamethylene diamine or other bifunctional molecules of similar size) for attachment of peptides.

Additionally, the L. intracellularis proteins and multiple antigen fusion molecules can be fused to either the carboxyl or amino terminals or both of the carrier molecule, or at sites internal to the carrier.

Carriers can be physically conjugated to the proteins of interest, using standard coupling reactions. Alternatively, chimeric molecules can be prepared recombinantly for use in the present invention, such as by fusing a gene encoding a suitable polypeptide carrier to one or more copies of a gene, or fragment thereof, encoding for selected L. intracellularis proteins or L. intracellularis multiple epitope fusion molecules.

Preferably, the above-described antigens, fusions and carrier conjugates, are produced recombinantly. A polynucleotide encoding these proteins can be introduced into an expression vector which can be expressed in a suitable expression system. A variety of bacterial, yeast, mammalian and insect expression systems are available in the art and any such expression system can be used. Optionally, a polynucleotide encoding these proteins can be translated in a cell-free translation system. Such methods are well known in the art. The proteins also can be constructed by solid phase protein synthesis.

B. L. intracellularis Polynucleotides

L. intracellularis polynucleotides encoding the L. intracellularis antigens for use in the subject compositions can be derived from any L. intracellularis strain or isolate.

Representative polynucleotide sequences encoding the L. intracellularis antigens are shown in SEQ ID Nos: 1, 4, 7, 10, 13, 16, 19, 22, 25, 28 and 31 (FIGS. 1A, 2A, 3A, 4A, 5A, 6A, 7A, 8A, 9A, 10A, and 11A, respectively). The polynucleotides can be modified for expression in a particular host cell, such as E coli.

The polynucleotide sequences encoding L. intracellularis antigens will encode the full-length amino acid sequences, or fragments or variants of these sequences so long as the resulting antigens stimulate an immunological response, preferably, a protective immune response. Thus, the polynucleotides can encode antigens with deletions or additions, as described above.

Once the coding sequences for the desired antigens have been isolated or synthesized, they can be cloned into any suitable vector or replicon for expression. Numerous cloning vectors are known to those of skill in the art, and the selection of an appropriate cloning vector is a matter of choice. A variety of bacterial, yeast, plant, mammalian and insect expression systems are available in the art and any such expression system can be used. Optionally, a polynucleotide encoding these proteins can be translated in a cell-free translation system. Such methods are well known in the art.

Examples of recombinant DNA vectors for cloning and host cells which they can transform include the bacteriophage λ (E. coli), pBR322 (E. coli), pACYC177 (E. coli), pKT230 (gram-negative bacteria), pGV1106 (gram-negative bacteria), pLAFR1 (gram-negative bacteria), pME290 (non-E. coli gram-negative bacteria), pHV14 (E. coli and Bacillus subtilis), pBD9 (Bacillus), pIJ61 (Streptomyces), pUC6 (Streptomyces), YIp5 (Saccharomyces), YCp19 (Saccharomyces) and bovine papilloma virus (mammalian cells). See, generally, Sambrook et al., Molecular Cloning, a laboratory manual, Cold Spring Harbor Laboratories, New York.

Insect cell expression systems, such as baculovirus systems, can also be used and are known to those of skill in the art. Plant expression systems can also be used to produce the immunogenic proteins. Generally, such systems use virus-based vectors to transfect plant cells with heterologous genes.

Viral systems, such as a vaccinia based infection/transfection system, will also find use with the present invention. In this system, cells are first transfected in vitro with a vaccinia virus recombinant that encodes the bacteriophage T7 RNA polymerase. This polymerase displays exquisite specificity in that it only transcribes templates bearing T7 promoters. Following infection, cells are transfected with the DNA of interest, driven by a T7 promoter. The polymerase expressed in the cytoplasm from the vaccinia virus recombinant transcribes the transfected DNA into RNA which is then translated into protein by the host translational machinery. The method provides for high level, transient, cytoplasmic production of large quantities of RNA and its translation product(s).

The coding sequence can be placed under the control of a promoter, ribosome binding site (for bacterial expression) and, optionally, an operator (collectively referred to herein as “control elements”), so that the DNA sequence encoding the desired antigen is transcribed into RNA in the host cell transformed by a vector containing this expression construction. The coding sequence may or may not contain a signal peptide or leader sequence. Leader sequences can be removed by the host in post-translational processing.

Other regulatory sequences may also be desirable which allow for regulation of expression of the protein sequences relative to the growth of the host cell. Such regulatory sequences are known to those of skill in the art, and examples include those which cause the expression of a gene to be turned on or off in response to a chemical or physical stimulus, including the presence of a regulatory compound. Other types of regulatory elements may also be present in the vector, for example, enhancer sequences.

The control sequences and other regulatory sequences may be ligated to the coding sequence prior to insertion into a vector. Alternatively, the coding sequence can be cloned directly into an expression vector which already contains the control sequences and an appropriate restriction site.

In some cases it may be necessary to modify the coding sequence so that it may be attached to the control sequences with the appropriate orientation; i.e., to maintain the proper reading frame. It may also be desirable to produce mutants or analogs of the immunogenic proteins. Mutants or analogs may be prepared by the deletion of a portion of the sequence encoding the protein, by insertion of a sequence, and/or by substitution of one or more nucleotides within the sequence. Techniques for modifying nucleotide sequences, such as site-directed mutagenesis, are well known to those skilled in the art. See, e.g., Sambrook et al., Molecular Cloning, a laboratory manual, Cold Spring Harbor Laboratories, New York.

The expression vector is then used to transform an appropriate host cell. A number of mammalian cell lines are known in the art and include immortalized cell lines available from the American Type Culture Collection (ATCC), such as, but not limited to, Chinese hamster ovary (CHO) cells, HeLa cells, baby hamster kidney (BHK) cells, monkey kidney cells (COS), human hepatocellular carcinoma cells (e.g., Hep G2), as well as others. Similarly, bacterial hosts such as E. coli, Bacillus subtilis, and Streptococcus spp., will find use with the present expression constructs. Yeast hosts useful in the present invention include inter alia, Saccharomyces cerevisiae, Candida albicans, Candida maltosa, Hansenula polymorpha, Kluyveromyces fragilis, Kluyveromyces lactis, Pichia guillerimondii, Pichia pastoris, Schizosaccharomyces pombe and Yarrowia lipolytica. Insect cells for use with baculovirus expression vectors include, inter alia, Aedes aegypti, Autographa californica, Bombyx mori, Drosophila melanogaster, Spodoptera frupperda, and Trichoplusia ni.

Depending on the expression system and host selected, the proteins of the present invention are produced by growing host cells transformed by an expression vector described above under conditions whereby the protein of interest is expressed. The selection of the appropriate growth conditions is within the skill of the art. If the proteins are not secreted, the cells are then disrupted, using chemical, physical or mechanical means, which lyse the cells yet keep the proteins substantially intact. Following disruption of the cells, cellular debris is removed, generally by centrifugation. Whether produced intracellularly or secreted, the protein can be further purified, using standard purification techniques such as but not limited to, column chromatography, ion-exchange chromatography, size-exclusion chromatography, electrophoresis, high-performance liquid chromatography (HPLC), immunoabsorbent techniques, affinity chromatography, immunoprecipitation, and the like.

### C. Antibodies

The antigens of the present invention can be used to produce antibodies for therapeutic (e.g., passive immunization), diagnostic and purification purposes. These antibodies may be polyclonal or monoclonal antibody preparations, monospecific antisera, or may be hybrid or chimeric antibodies, such as humanized antibodies, altered antibodies, F(ab′)2 fragments, F(ab) fragments, Fv fragments, single-domain antibodies, dimeric or trimeric antibody fragment constructs, minibodies, or functional fragments thereof which bind to the antigen in question. Antibodies are produced using techniques well known to those of skill in the art.

For subjects known to have a L. intracellularis-related disease, an anti-L. intracellularis-antigen antibody may have therapeutic benefit and can be used to confer passive immunity to the subject in question. Alternatively, antibodies can be used in diagnostic applications, described further below, as well as for purification of the antigen of interest.

### D. Compositions

The L. intracellularis molecules can be formulated into compositions for delivery to subjects for eliciting an immune response, such as for inhibiting infection. Compositions of the invention may comprise or be co-administered with non-L. intracellularis antigens or with a combination of L. intracellularis antigens, as described above. Methods of preparing such formulations are described in, e.g., Remington's Pharmaceutical Sciences, Mack Publishing Company, Easton, Pennsylvania, 22nd Edition, 2012. The compositions of the present invention can be prepared as injectables, either as liquid solutions or suspensions. Solid forms suitable for solution in or suspension in liquid vehicles prior to injection may also be prepared. The preparation may also be emulsified or the active ingredient encapsulated in liposome vehicles. The active immunogenic ingredient is generally mixed with a compatible pharmaceutical vehicle, such as, for example, water, saline, dextrose, glycerol, ethanol, or the like, and combinations thereof. In addition, if desired, the vehicle may contain minor amounts of auxiliary substances such as wetting or emulsifying agents and pH buffering agents.

Adjuvants which enhance the effectiveness of the composition may also be added to the formulation. Such adjuvants include any compound or combination of compounds that act to increase an immune response to a L. intracellularis antigen or combination of antigens, thus reducing the quantity of antigen necessary in the vaccine, and/or the frequency of injection necessary in order to generate an adequate immune response.

For example, a triple adjuvant formulation as described in, e.g., U.S. Pat. No. 9,061,001, incorporated herein by reference in its entirety, can be used in the subject compositions. The triple adjuvant formulation includes a host defense peptide, in combination with a polyanionic polymer such as a polyphosphazene, and a nucleic acid sequence possessing immunostimulatory properties (ISS), such as an oligodeoxynucleotide molecule with or without a CpG motif (a cytosine followed by guanosine and linked by a phosphate bond) or the synthetic dsRNA analog poly(I:C).

Examples of host defense peptides for use in the combination adjuvant, as well as individually with the antigen include, without limitation, HH2 (VQLRIRVAVIRA, SEQ ID NO: 34); 1002 (VQRWLIVWRIRK, SEQ ID NO: 35); 1018 (VRLIVAVRIWRR, SEQ ID NO: 36); Indolicidin (ILPWKWPWWPWRR, SEQ ID NO: 37); HH111 (ILKWKWPWWPWRR, SEQ ID NO: 38); HH113 (ILPWKKPWWPWRR, SEQ ID NO: 39); HH970 (ILKWKWPWWKWRR, SEQ ID NO: 40); HH1010 (ILRWKWRWWRWRR, SEQ ID NO: 41); Nisin Z (Ile-Dhb-Ala-Ile-Dha-Leu-Ala-Abu-Pro-Gly-Ala-Lys-Abu-Gly-Ala-Leu-Met-Gly-Ala-Asn-Met-Lys-Abu-Ala-Abu-Ala-Asn-Ala-Ser-Ile-Asn-Val-Dha-Lys, SEQ ID NO: 42); JK1 (VFLRRIRVIVIR, SEQ ID NO: 43); JK2 (VFWRRIRVWVIR, SEQ ID NO: 44); JK3 (VQLRAIRVRVIR, SEQ ID NO: 45); JK4 (VQLRRIRVWVIR, SEQ ID NO: 46); JK5 (VQWRAIRVRVIR, SEQ ID NO: 47); and JK6 (VQWRRIRVWVIR, SEQ ID NO: 48). Any of the above peptides, as well as fragments and analogs thereof, that display the appropriate biological activity, such as the ability to modulate an immune response, such as to enhance an immune response to a co-delivered antigen, will find use herein.

Exemplary, non-limiting examples of ISSs for use in the triple adjuvant composition, or individually include, CpG oligonucleotides or non-CpG molecules. By “CpG oligonucleotide” or “CpG ODN” is meant an immunostimulatory nucleic acid containing at least one cytosine-guanine dinucleotide sequence (i.e., a 5′ cytidine followed by 3′ guanosine and linked by a phosphate bond) and which activates the immune system. An “unmethylated CpG oligonucleotide” is a nucleic acid molecule which contains an unmethylated cytosine-guanine dinucleotide sequence (i.e., an unmethylated 5′ cytidine followed by 3′ guanosine and linked by a phosphate bond) and which activates the immune system. A “methylated CpG oligonucleotide” is a nucleic acid which contains a methylated cytosine-guanine dinucleotide sequence (i.e., a methylated 5′ cytidine followed by a 3′ guanosine and linked by a phosphate bond) and which activates the immune system. CpG oligonucleotides are well known in the art and described in, e.g., U.S. Pat. Nos. 6,194,388; 6,207,646; 6,214,806; 6,218,371; 6,239,116; and 6,339,068; PCT Publication No. WO 01/22990; PCT Publication No. WO 03/015711; US Publication No. 20030139364, which patents and publications are incorporated herein by reference in their entireties.

Examples of such CpG oligonucleotides include, without limitation, 5′ TCCATGACGTTCCTGACGTT3′, termed CpG ODN 1826 (SEQ ID NO: 49), a Class B CpG; 5′TCGTCGTTGTCGTTTTGTCGTT3′, termed CpG ODN 2007 (SEQ ID NO: 50), a Class B CpG; 5′ TCGTCGTTTTGTCGTTTTGTCGTT3′, also termed CPG 7909 or 10103 (SEQ ID NO: 51), a Class B CpG; 5′ GGGGACGACGTCGTGGGGGGG 3′, termed CpG 8954 (SEQ ID NO: 52), a Class A CpG; and 5′ TCGTCGTTTTCGGCGCGCGCCG 3′, also termed CpG 2395 or CpG 10101 (SEQ ID NO: 53), a Class C CpG. All of the foregoing class B and C molecules are fully phosphorothioated.

Non-CpG oligonucleotides for use in the present composition include the double stranded polyriboinosinic acid:polyribocytidylic acid, also termed poly(I:C); and a non-CpG oligonucleotide 5′AAAAAAGGTACCTAAATAGTATGTTTCTGAAA3′ (SEQ ID NO: 54).

Polyanionic polymers for use in the triple combination adjuvants or alone include polyphosphazenes (sometimes termed “polyphosphazines”). Typically, polyphosphazenes for use with the present adjuvant compositions will either take the form of a polymer in aqueous solution or a polymer microparticle, with or without encapsulated or adsorbed substances such as antigens or other adjuvants. For example, the polyphosphazene can be a soluble polyphosphazene, such as a polyphosphazene polyelectrolyte with ionized or ionizable pendant groups that contain, for example, carboxylic acid, sulfonic acid or hydroxyl moieties, and pendant groups that are susceptible to hydrolysis under conditions of use to impart biodegradable properties to the polymer. Such polyphosphazene polyelectrolytes are well known and described in, for example, U.S. Pat. Nos. 5,494,673; 5,562,909; 5,855,895; 6,015,563; and 6,261,573, incorporated herein by reference in their entireties. Alternatively, polyphosphazene polymers in the form of cross-linked microparticles will also find use herein. Such cross-linked polyphosphazene polymer microparticles are well known in the art and described in, e.g., U.S. Pat. Nos. 5,053,451; 5,149,543; 5,308,701; 5,494,682; 5,529,777; 5,807,757; 5,985,354; and 6,207,171, incorporated herein by reference in their entireties.

Examples of particular polyphosphazene polymers for use herein include poly[di(sodium carboxylatophenoxy)phosphazene] (PCPP) and poly(di-4-oxyphenylproprionate)phosphazene (PCEP), in various forms, such as the sodium salt, or acidic forms, as well as a polymer composed of varying percentages of PCPP or PCEP copolymer with hydroxyl groups, such as 90:10 PCPP/OH. Cyclic or linear polyphosphazenes may be used in compositions described herein. Methods for synthesizing these compounds are known and described in the patents referenced above, as well as in Andrianov et al., Biomacromolecules (2004) 5:1999; Andrianov et al., Macromolecules (2004) 37:414; Mutwiri et al., Vaccine (2007) 25:1204. Contemplated cyclic polyphosphazenes may include those found in: Cyclopolyphosphazenes, related methods of preparation and methods of use, U.S. provisional application no: YYY.

Additional adjuvants include alum, chitosan-based adjuvants, and any of the various saponins, oils, and other substances known in the art, such as AMPHIGEN™ which comprises de-oiled lecithin dissolved in an oil, usually light liquid paraffin. In vaccine preparations AMPHIGEN™ is dispersed in an aqueous solution or suspension of the immunizing antigen as an oil-in-water emulsion. Other adjuvants are LPS, bacterial cell wall extracts, bacterial DNA, synthetic oligonucleotides and combinations thereof (Schijns et al., Curr. Opi. Immunol. (2000) 12:456), Mycobacterial phlei (M. phlei) cell wall extract (MCWE) (U.S. Pat. No. 4,744,984), M. phlei DNA (M-DNA), and M-DNA-M. phlei cell wall complex (MCC). For example, compounds which may serve as emulsifiers herein include natural and synthetic emulsifying agents, as well as anionic, cationic and nonionic compounds. Among the synthetic compounds, anionic emulsifying agents include, for example, the potassium, sodium and ammonium salts of lauric and oleic acid, the calcium, magnesium and aluminum salts of fatty acids (i.e., metallic soaps), and organic sulfonates such as sodium lauryl sulfate. Synthetic cationic agents include, for example, cetyltrimethylammonium bromide, while synthetic nonionic agents are exemplified by glyceryl esters (e.g., glyceryl monostearate), polyoxyethylene glycol esters and ethers, and the sorbitan fatty acid esters (e.g., sorbitan monopalmitate) and their polyoxyethylene derivatives (e.g., polyoxyethylene sorbitan monopalmitate). Natural emulsifying agents include acacia, gelatin, lecithin and cholesterol.

Other suitable adjuvants can be formed with an oil component, such as a single oil, a mixture of oils, a water-in-oil emulsion, or an oil-in-water emulsion. The oil may be a mineral oil, a vegetable oil, or an animal oil. Mineral oil, or oil-in-water emulsions in which the oil component is mineral oil are preferred. Another oil component are the oil-in-water emulsions sold under the trade name of EMULSIGEN®, such as but not limited to EMULSIGEN PLUS®, comprising a light mineral oil as well as 0.05% formalin, and 30 μg/mL gentamicin as preservatives, available from MVP Laboratories, Ralston, NE. Also of use herein is an adjuvant known as “VSA3” which is a modified form of EMULSIGEN PLUS® which includes DDA (See, U.S. Pat. No. 5,951,988, incorporated herein by reference in its entirety). The adjuvant MONTANIDE™ will also find use herein. Suitable animal oils include, for example, cod liver oil, halibut oil, menhaden oil, orange roughy oil and shark liver oil, all of which are available commercially. Suitable vegetable oils, include, without limitation, canola oil, almond oil, cottonseed oil, corn oil, olive oil, peanut oil, safflower oil, sesame oil, soybean oil, and the like.

Alternatively, a number of aliphatic nitrogenous bases can be used as adjuvants with the vaccine formulations. For example, known immunologic adjuvants include amines, quaternary ammonium compounds, guanidines, benzamidines and thiouroniums (Gall, D. (1966) Immunology 11:369 386). Specific compounds include dimethyldioctadecylammonium bromide (DDA) (available from Kodak) and N,N-dioctadecyl-N,N-bis(2-hydroxyethyl)propanediamine (“AVRIDINE”). The use of DDA as an immunologic adjuvant has been described; See, e.g., the Kodak Laboratory Chemicals Bulletin 56(1):1 5 (1986); Adv. Drug Deliv. Rev. 5(3):163 187 (1990); J. Controlled Release 7:123 132 (1988); Clin. Exp. Immunol. 78(2):256 262 (1989); J. Immunol. Methods 97(2):159 164 (1987); Immunology 58(2):245 250 (1986); and Int. Arch. Allergy Appl. Immunol. 68(3):201 208 (1982). AVRIDINE is also a well-known adjuvant. See, e.g., U.S. Pat. No. 4,310,550, incorporated herein by reference in its entirety, which describes the use of N,N-higher alkyl-N′,N′-bis(2-hydroxyethyl)propane diamines in general, and AVRIDINE in particular, as vaccine adjuvants. U.S. Pat. No. 5,151,267 to Babiuk, incorporated herein by reference in its entirety, and Babiuk et al. (1986) Virology 159:57 66, also relate to the use of AVRIDINE as a vaccine adjuvant.

In some cases, the formulations may comprise a mucoadhesive lipidic carrier system, such as those known in the art, for example PCT application serial no. PCT/CA2019/051347, titled Mucoadhesive Lipidic Delivery System, which is herein incorporated by reference. The mucoadhesive lipidic carrier system may enhance an immune response to a selected antigen when administered by a suitable method, such as mucosally or intramuscularly. In certain embodiments, the mucoadhesive lipidic carrier comprises a cationic liposome, such as, but not limited to, a mucoadhesive cationic lipid carrier comprising one or more cationic lipids selected from 1,2-dioleoyl-3-trimethylammonium-propane (DOTAP); 3β-[N—(N′,N′-dimethylaminoethane)-carbamoyl] (DC); dimethyldioctadecylammonium (DDA); octadecylamine (SA); dimethyldioctadecylammonium bromide (DDAB); 1,2-dioleoyl-sn-glycerol-3-phosphoethanolamine (DOPE); egg L-α-phosphatidylcholine (EPC); cholesterol (Chol); distearoylphosphatidylcholine (DSPC); 1,2-dimyristoyl-3-trimethylammonium-propane (DMTAP); dimyristoylphosphatidylcholine (DMPC); or ceramide carbamoyl-spermine (CCS).

Once prepared, the formulations will contain a “pharmaceutically effective amount” of the active ingredient, that is, an amount capable of achieving the desired response in a subject to which the composition is administered. In the treatment and prevention of a L. intracellularis disease, a “pharmaceutically effective amount” would preferably be an amount which prevents, reduces or ameliorates the symptoms of the disease in question. The exact amount is readily determined by one skilled in the art using standard tests. The active ingredient will typically range from about 1% to about 95% (w/w) of the composition, or even higher or lower if appropriate. With the present formulations, 1 μg to 2 mg, such as 10 μg to 1 mg, e.g., 25 μg to 0.5 mg, 50 μg to 200 μg, or any values between these ranges of active ingredient per mL of injected solution should be adequate to treat or prevent infection when a dose of 1 to 5 mL per subject is administered. The quantity to be administered depends on the subject to be treated, the capacity of the subject's immune system to synthesize antibodies, and the degree of protection desired. Effective dosages can be readily established by one of ordinary skill in the art through routine trials establishing dose response curves.

The composition can be administered parenterally, e.g., by intratracheal, intramuscular, subcutaneous, intraperitoneal, or intravenous injection. The subject is administered at least one dose of the composition. Moreover, the subject may be administered as many doses as is required to bring about the desired biological effect.

Additional formulations which are suitable for other modes of administration include suppositories and, in some cases, aerosol, mucosal such as intranasal, oral formulations, and sustained release formulations. For suppositories, the vehicle composition will include traditional binders and carriers, such as, polyalkaline glycols, or triglycerides. Such suppositories may be formed from mixtures containing the active ingredient in the range of about 0.5% to about 10% (w/w), preferably about 1% to about 2%. Oral vehicles include such normally employed excipients as, for example, pharmaceutical grades of mannitol, lactose, starch, magnesium, stearate, sodium saccharin cellulose, magnesium carbonate, and the like. These oral vaccine compositions may be taken in the form of solutions, suspensions, tablets, pills, capsules, sustained release formulations, or powders, and contain from about 10% to about 95% of the active ingredient, preferably about 25% to about 70%.

Mucosal formulations, such as intranasal, intravaginal, intrarectal and intrauterine formulations, will usually include vehicles that limit irritation to the mucosa. In cases of intranasal formulations, the vehicles may either not irritate the mucosa nor significantly disturb ciliary function. Diluents such as water, aqueous saline or other known substances can be employed with the subject invention. The nasal formulations may also contain preservatives such as, but not limited to, chlorobutanol and benzalkonium chloride. A surfactant may be present to enhance absorption of the subject antigens by the nasal mucosa.

Controlled or sustained release formulations are made by incorporating the antigen into carriers or vehicles such as liposomes, nonresorbable impermeable polymers such as ethylenevinyl acetate copolymers and HYTREL copolymers, swellable polymers such as hydrogels, resorbable polymers such as collagen and certain polyacids or polyesters such as those used to make resorbable sutures, polyphosphazenes, alginate, microparticles, gelatin nanospheres, chitosan nanoparticles, and the like. The antigens described herein can also be delivered using implanted mini-pumps, well known in the art.

The vaccine can be administered to nursing animals, such as nursing piglets, weaner piglets, growers or gilts/sows. The vaccine can also be administered to foals, mares, boars or stallions, or any of the other various species described herein.

Prime-boost methods can be employed where one or more compositions are delivered in a “priming” step and, subsequently, one or more compositions are delivered in a “boosting” step. In certain embodiments, priming and boosting with one or more compositions described herein is followed by additional boosting. The compositions delivered can include the same antigens, or different antigens, given in any order and via any administration route.

### E. Tests to Determine the Efficacy of an Immune Response

One way of assessing efficacy of therapeutic treatment involves monitoring infection after administration of a composition of the invention. One way of assessing efficacy of prophylactic treatment involves monitoring immune responses against the L. intracellularis antigens in the compositions of the invention after administration of the composition. Another way of assessing the immunogenicity of the immunogenic compositions of the present invention is to screen the subject's sera by immunoblot. A positive reaction indicates that the subject has previously mounted an immune response to the L. intracellularis antigens, that is, the L. intracellularis protein is an immunogen. This method may also be used to identify epitopes.

Another way of checking efficacy of therapeutic treatment involves monitoring infection after administration of the compositions of the invention. One way of checking efficacy of prophylactic treatment involves monitoring immune responses both systemically (such as monitoring the level of IgG1 and IgG2a production) and mucosally (such as monitoring the level of IgA production) against the antigens in the compositions of the invention after administration of the composition. Typically, serum-specific antibody responses are determined post-immunization but pre-challenge, whereas mucosal-specific antibody responses are determined post-immunization and post-challenge.

The immunogenic compositions of the present invention can be evaluated in in vitro and in vivo animal models prior to host administration.

The efficacy of immunogenic compositions of the invention can also be determined in vivo by challenging animal models of infection with the immunogenic compositions. The immunogenic compositions may or may not be derived from the same strains as the challenge strains. Preferably the immunogenic compositions are derivable from the same strains as the challenge strains.

The immune response may be one or both of a TH1-type immune response and a TH2-type response. The immune response may be an improved or an enhanced or an altered immune response. The immune response may be one or both of a systemic and a mucosal immune response. An enhanced systemic and/or mucosal immunity is reflected in an enhanced TH1-type and/or TH2-type immune response. Preferably, the enhanced immune response includes an increase in the production of IgG1 and/or IgG2a and/or IgA. Preferably the mucosal immune response is a TH2-type immune response. Preferably, the mucosal immune response includes an increase in the production of IgA.

Activated TH2 cells enhance antibody production and are therefore of value in responding to extracellular infections. Activated TH2-type cells may secrete one or more of IL-4, IL-5, IL-6, and IL-10. A TH2 immune response may result in the production of IgG1, IgE, IgA and memory B cells for future protection.

A TH1-type immune response may include one or more of an increase in CTLs, an increase in one or more of the cytokines associated with a TH1-type immune response (such as IL-2, IFNγ, and TNFβ), an increase in activated macrophages, an increase in NK activity, or an increase in the production of IgG2a. Preferably, the enhanced TH1-type immune response will include an increase in IgG2a production.

The immunogenic compositions of the invention will preferably induce long lasting immunity that can quickly respond upon exposure to one or more infectious antigens.

### F. Kits

The invention also provides kits comprising one or more containers of compositions of the invention. Compositions can be in liquid form or can be lyophilized, as can individual antigens. Suitable containers for the compositions include, for example, bottles, vials, syringes, and test tubes. Containers can be formed from a variety of materials, including glass or plastic. A container may have a sterile access port (for example, the container may be an intravenous solution bag or a vial having a stopper pierceable by a hypodermic injection needle).

The kit can further comprise a second container comprising a pharmaceutically-acceptable buffer, such as phosphate-buffered saline, Ringer's solution, or dextrose solution. It can also contain other materials useful to the end-user, including other pharmaceutically acceptable formulating solutions such as buffers, diluents, filters, needles, and syringes or other delivery device. The kit may further include a third component comprising an adjuvant.

The kit can also comprise a package insert containing written or computer-readable instructions for methods of inducing immunity or for treating infections. The package insert can be an unapproved draft package insert or can be a package insert approved by the Food and Drug Administration (FDA) or other regulatory body.

The invention also provides a delivery device pre-filled with the immunogenic compositions of the invention.

Similarly, antibodies can be provided in kits, with suitable instructions and other necessary reagents. The kit can also contain, depending on if the antibodies are to be used in immunoassays, suitable labels and other packaged reagents and materials (i.e. wash buffers and the like). Standard immunoassays can be conducted using these kits.

### 3. EXPERIMENTAL

Below are examples of specific embodiments for carrying out the present invention. The examples are offered for illustrative purposes only, and are not intended to limit the scope of the present invention in any way.

Efforts have been made to ensure accuracy with respect to numbers used (e.g., amounts, temperatures, etc.), but some experimental error and deviation should, of course, be allowed for.

### Materials and Methods

### Cell Culture Conditions:

Undifferentiated porcine intestinal epithelial cell lines (IPEC-1), derived from the jejunum and ileum of unsuckled one-day-old piglets (Cano et al., PLOS One (2013) 8:e53647), were cultured and maintained in DMEM/F-12 (SH30271.01; HyClone™ (Thermo Fisher Scientific, San Jose, CA, USA)) with 5% Fetal bovine serum (FBS) (Sigma-Aldrich, Oakville, ON, Canada); penicillin/streptomycin (Gibco 5000 units/mL Penicillin, 5000 μg/mL Streptomycin); insulin (10 μg/mL); transferrin (5.5 μg/mL); selenium (5 ng/mL) (ITS; Sigma-Aldrich, Oakville, ON, Canada)) and 5 ng/mL of epidermal growth factor (Sigma-Aldrich, Oakville, ON, Canada). Cells were kept in humidified incubator in an atmosphere of 5% CO₂ and 95% air at 37° C. and passaged two times per week at 1:5 ratio in Corning 75 cm² cell culture flasks. IPEC-1 cells used for L. intracellularis infection and neutralization assays were grown as indicated above but in the absence of antibiotics.

L. intracellularis Protein Sample Preparation:

L. intracellularis pellets prepared from infected McCoy cells (detailed in Lawson et al., J. Clin. Microbiol. (1993) 31:1136-1142) were resuspended in radioimmunoprecipitation assay (RIPA) buffer (0.05 M Tris pH 8, Bio Basic Canada INC, Markham. ON, Canada); 0.15 M Sodium Chloride (Bio Basic Canada INC.); 0.10% SDS, (Bio Basic Canada INC.); 1% Deoxycholic acid (VWR-Amresco, Dublin, Ireland); 1% Nonidet P40 substitute (Sigma-Aldrich); distilled water) complete with 0.1 M PMSF (Sigma-Aldrich) in isopropanol (Sigma-Aldrich). Samples were frozen/thawed three times to rupture the cells and the mixture was centrifuged at 10,000×g for 10 minutes. Bacterial proteins were then precipitated from the supernatant with ice-cold acetone. The mixture was vortexed and stored in −20° C. for 1 hour then the incubation mixture was centrifuged at 14,000×g for 10 minutes. The supernatant was carefully discarded and the pellet dried before resuspending with NaHCO₃ buffer and quantifying by bicinchoninic acid (BCA) analysis, following the manufacturer's instructions (Pierce, Thermo Fisher Scientific).

L. intracellularis proteins were labelled with Cy5 dye (GE Healthcare Life Sciences-Amersham Biosciences, Mississauga, ON, Canada) in a dye/protein molar ratio of 8:1, following the manufacturer's recommended protocols. The mixture was incubated for 4 hours at room temperature in the dark. Unbound dye was removed by size filtration using 3000 MWCO 15 mL volume filters (Millipore, Etobicoke, ON, Canada) with four additional washes. The final concentration of Cy5-labelled L. intracellularis proteins was determined by BCA assay (Pierce) prior to 2DE.

Binding of Cy5-Labeled L. intracellularis Proteins to IPEC Cells:

IPEC-1 cells were grown to confluence in T-75 flasks, tripsonized and washed 3 times with antibiotic- and FBS-free IPEC medium. Next, 1×10⁶ IPEC cells were incubated with 700 μg of Cy5-labelled bacterial proteins for 3 hours with gentle nutation at 4° C. Cells were centrifuged as indicated above and the unbound L. intracellularis proteins were removed with the supernatant. The IPEC-1 cells and bound L. intracellularis proteins were then resuspended in RIPA buffer with PMSF (Sigma-Aldrich) and subjected to repeated freeze/thaws as indicated above. IPEC-1 proteins and Cy5-labeled adherent L. intracellularis proteins were then subjected to two dimensional gel electrophoresis (2DE).

### 2-Dimensional Gel Electrophoresis:

Proteins from lysed IPEC-1 cells and bound Cy5-labeled L. intracellularis (250 μg analytical gels 600 μg for prep gels) were resuspended in rehydration buffer overnight (9 M urea, 2% CHAPS (Fisher BioReagents), 1% DTT (Promega, Medison, Wi, USA), 2% pharmalyte 5-8 (GE Healthcare), 0.002% bromophenol blue (BioRad, Hercules, CA, USA)) and were loaded onto an IPG strip (Immobiline™ DryStrip, pH 4-7, 13 cm, GE Healthcare). The strips were individually subjected to isoelectric focusing (IEF) using IPGphor™ device (GE Healthcare-Amersham Biosciences) using a stepwise protocol (150 V step and hold for 3 h, 300 V step and hold 1200 Vh, 1000 V gradient for 3900 Vh, 8000 V gradient for 13500 Vh and 8000 V step and hold for 25000 Vh). After IEF, both IPG strips were stored at −80° C. IPG strips with isoelectric focused proteins were thawed at room temperature and equilibrated with SDS equilibration buffer with 1% DTT (6 M urea, 75 mM Tris-HCl pH 8.8, 29.3% glycerol, 2% SDS and 0.002% bromophenol blue) for 15 minutes at room temperature, followed by washing with SDS equilibration buffer with 2.5% Iodoacetamide (GE Healthcare) for 15 minutes. After equilibration, strips were placed over SDS gels and covered with sealing solution (0.5 agarose in 1×SDS running buffer). Second dimension electrophoresis was performed using BIO-RAD protean II xi Cell Apparatus and two medium size, 10% SDS PAGE gels. Electrophoresis was performed using 90 V constant voltage for 16 hours with constant water cooling of the apparatus (Bio-Rad Power pack 200, Hercules, CA, USA).

### Western Blot Analysis and Silver Staining:

Proteins on the analytical gel were transferred with semi-dry transfer to a nitrocellulose membrane (BIO-RAD, 162-0094) using Bio-Rad Trans-Blot SD Semi-Dry™ transfer cell (15 V for 60 minutes) and then Western blot (WB) analysis was performed using rabbit hyperimmune serum (1:500; obtained from rabbits immunized with whole bacteria) as primary antibodies. Anti-rabbit IR 800 antibody (1 μg/mL; Li-COR, Lincoln, NE, USA) was used as secondary antibody. The membrane was scanned with Odyssey scanner (Li-COR) in the IR 700 and IR 800 channels. IR800-stained proteins are indicative of bacterial proteins with affinity for IPEC-1 cells and bound by rabbit serum against whole bacteria.

For the preparative gel, L. intracellularis proteins were stained with Silver stain kit, (PROTSIL-1-KT™, Sigma Aldrich) according to manufacturer's protocol and this gel was reserved for excising gel spots for Mass Spectrometry analysis.

### Preparation of Samples for Mass Spectrometry:

Silver-stained proteins on the preparative gel which correspond to IR-800-labelled proteins detected by WB analysis were excised from the gel using a sterile biopsy punch (3 mm diameter) to avoid contamination of gel samples with environmental proteins. Gel plugs were collected, and stored in ultrapure water at −20° C. Gel plug samples (annotated as 1.4, 2.3, 3.1, 3.2 and 4) were sent to Plateforme Protéomique Centre de Recherche du CHU de Québec CHUL, Québec, Canada for Mass Spectrometry (MS) analysis.

### Tryptic Digest:

Protein digestion and MS analyses were performed by the Proteomics Platform of the CHU de Québec Research Center (Quebec, Canada). Excised gel pieces were placed in 96-well plates and then washed with water followed by tryptic digestion performed using a liquid handling robot (MultiProbe™, Perkin Elmer), according to the manufacturer's specifications. Briefly, proteins were reduced with 10 mM DTT and alkylated with 55 mM iodoacetamide. Trypsin digestion was performed using 126 nM of modified porcine trypsin (Sequencing grade, Promega, Madison, WI) at 37° C. for 18 hours. Digestion products were extracted using 1% formic acid, 2% acetonitrile followed by 1% formic acid, 50% acetonitrile. The recovered extracts were pooled, vacuum centrifuge dried and then resuspended into 12 μl of 0.1% formic acid and 5 μl were analyzed by MS.

### Mass Spectrometry:

Peptide samples were injected and separated by online reversed-phase (RP) nanoscale capillary liquid chromatography (nanoLC) and analyzed by electrospray mass spectrometry (ESI MS/MS). The experiments were performed with a Dionex UltiMate™ 3000 nanoRSLC chromatography system (Thermo Fisher Scientific/Dionex Softron GmbH, Germering, Germany) connected to an Orbitrap Fusion™ mass spectrometer (Thermo Fisher Scientific) driving with Orbitrap Fusion Tune Application 2.0 and equipped with a nanoelectrospray ion source. Peptides were trapped at 20 μL/min in loading solvent (2% acetonitrile, 0.05% TFA) on a 5 mm×300 μm C18 pepmap cartridge pre-column (Thermo Fisher Scientific/Dionex Softron GmbH, Germering, Germany) during 5 minutes. The pre-column was then switched online with a self-made 50 cm×75 μm internal diameter separation column packed with ReproSil-Pur C18-AQ 3-μm resin (Dr. Maisch HPLC GmbH, Ammerbuch-Entringen, Germany) and the peptides were eluted with a linear gradient from 5-40% solvent B (A: 0.1% formic acid, B: 80% acetonitrile, 0.1% formic acid) in 30 minutes at 300 nL/min. Mass spectra were acquired using a data dependent acquisition mode using Thermo XCalibur software version 3.0.63. Full scan mass spectra (350 to 1800 m/z) were acquired in the orbitrap using an AGC target of 4e5, a maximum injection time of 50 ms and a resolution of 120 000. Internal calibration using lock mass on the m/z 445.12003 siloxane ion was used. Each MS scan was followed by acquisition of fragmentation MS/MS spectra of the most intense ions for a total cycle time of 3 seconds (top speed mode). The selected ions were isolated using the quadrupole analyzer in a window of 1.6 m/z and fragmented by Higher Energy Collision-induced Dissociation (HCD) with 35% of collision energy. The resulting fragments were detected by the linear ion trap in rapid scan rate with an AGC target of 1e4 and a maximum injection time of 50 ms. Dynamic exclusion of previously fragmented peptides was set for a period of 20 seconds and a tolerance of 10 ppm.

### Database Searching:

All MS/MS samples were analyzed using Mascot (Matrix Science, London, UK; version 2.5.1). Mascot was set up to search the TAX_Desulfovibrio_CI_194924_20160714 database (104802 entries) assuming digestion with trypsin. Mascot was searched with a fragment ion mass tolerance of 0.60 Da and a parent ion tolerance of 10 ppm. Carbamidomethyl of cysteine was specified in Mascot as a fixed modification. Deamidated asparagine and glutamine and oxidation of methionine were specified in Mascot as variable modifications. Two missed cleavages were allowed.

### Criteria for Protein Identification:

Scaffold (version Scaffold 4.7.5, Proteome Software Inc., Portland, OR) was used to validate MS/MS-based peptide and protein identifications. Peptide identifications were accepted if they could be established at greater than 95.0% probability by the Peptide Prophet algorithm (Keller et al., Anal. Chem. (20012)74:5383-5392) with Scaffold delta-mass correction. Protein identifications were accepted if they could be established at greater than 95.0% probability and contained at least 2 identified peptides. Protein probabilities were assigned by the Protein Prophet algorithm (Nesvizhskii et al., Anal. Chem. (2003) 75:4646-4658). Proteins that contain similar peptides and could not be differentiated based on MS/MS analysis alone were grouped to satisfy the principles of parsimony.

### Bioinformatics Analysis of Proteins:

Amino acid sequences from peptides identified by mass spectrometry were submitted to BLAST algorithm (Altschul et al. Nucl. Acids Res. (1997) 25:3389-3402) to identify corresponding proteins. Prediction of functional domains and motifs was performed using UniProt (uniprot.org) and Pfam (pfam.xfam.org) and proteins are listed in Table 2. To compute physical and chemical parameters of MS/MS detected proteins, protein sequences were submitted to ExPasy ProtParam tool (web.expasy.org/protparam) (Gasteiger et al. in The Proteomics Protocols Handbook, Walker, J. M. (Ed.) Humana Press, Totowa, NJ (2005) pp. 571-607).

TABLE 2
L. intracellularis proteins detected by MS

| PHE/MN1-00 | NCBI | | Sequence | # | | | Isoelectric |
|---|---|---|---|---|---|---|---|
| Locus | PHE/MNI-00 | | coverage | Peptides | Probability | MW | Point |
| tag | Annotation | Spot | % | identified | % | (kDa) | (ExPasy) |
| LI0710 | Flagellin | 2.3 | 36 | 9 | 100 | 31.0 | 5.97 |
| LI0649 | Autotransporter | 4 | 4 | 4 | 100 | 91.9 | 4.81 |
| LI0169 | ABC type dipeptide | 4 | 4 | 2 | 100 | 63.6 | 6.52 |
| | transport system | | | | | | |
| LI1153 | Putative outer | 3.1 | 7 | 2 | 100 | 44.0 | 4.62 |
| | protein N | | | | | | |
| LI0786 | DNA polymerase III | 3.2 | 13 | 6 | 100 | 43.6 | 4.70 |
| | subunit B | | | | | | |
| LI1171 | 5′- | 4 | 11 | 5 | 100 | 62.3 | 5.71 |
| | nucleotidase/2′,3′- | | | | | | |
| | cyclic | | | | | | |
| | phosphodiesterase | | | | | | |
| | and related | | | | | | |
| | esterases | | | | | | |
| LI0608 | Cysteine-tRNA | 4 | 5 | 2 | 100 | 55.5 | 5.55 |
| | ligase | | | | | | |
| LI0726 | S- | 2.3 | 6 | 2 | 100 | 44.4 | 5.41 |
| | adenosylmethionine | | | | | | |
| | synthase | | | | | | |
| LI0823 | Xaa-Pro- | 2.3 | 5 | 2 | 100 | 41.0 | 5.60 |
| | aminopeptidase | | | | | | |
| LI0625 | 60 kDa chaperonin, | 3.2 | 5 | 2 | 100 | 58.6 | 5.63 |
| | groL | | | | | | |
| LI0794 | ATP-dependant Clp | 1.4 | 21 | 4 | 100 | 23.5 | 4.73 |
| | protease proteolytic | | | | | | |
| | subunit | | | | | | |

### Molecular Cloning:

To construct the proteins for expression, bacterial genomic DNA from avirulent L. intracellularis N343 was isolated using GenElute™ Bacterial Genomic DNA kit (Sigma-Aldrich) following the manufacturer's protocol. PCR amplification of open reading frames was performed using Phusion™ High-Fidelity PCR kit (New England Biosciences (NEB), Ispwitch, MA, USA). Primers with cleavage sites for in-frame cloning with the N-terminal His-tag contained within the expression vector pET30a which was used for LI0169 and LI0649 and pET30a.1 which was used for the other remaining genes. Both plasmids are IPTG inducible, T7 expression vectors, C-terminal His tag (Novagen/Millapore Sigma, Burlington, MA, USA). Primers used to clone each gene are listed in Table 3. These were based on the genomic sequence of L. intracellularis (PHE/MN1-00). DNA was gel purified, cut with the appropriate restriction enzymes (either BamHI/XhoI or NcoI/XhoI) and ligated into pET30a using T4 DNA Ligase (NEB, Ispwitch, MA, USA). The resulting constructs were transformed into competent Dh5α E. coli using standard procedures. Stocks of the plasmid DNAs were isolated from the bacteria using Presto Mini plasmid kit (Genaid, New Taipei City, Taiwan). The cloned sequences and vector insertion were validated by DNA sequencing and restriction digests.

Recombinant L. intracellularis proteins included a His-tag with linker at the N-terminus for purification purposes. Additionally, N-terminal transmembrane domains were deleted from rLI0649 and rLI0169. The gene sequences and the expressed proteins that were cloned in frame with a His-tag sequence are shown in FIGS. 1-11.

TABLE 3
Primers

| SEQ | | | |
|---|---|---|---|
| ID | Primer | | Restriction |
| NO. | name | Sequence 5′ to 3′ | site |
| 55 | LI0710-F | GACGGATCCTCTCTTGTCATTAATAACAACCTGATGG | BamHI |
| 56 | LI0710-R | GAGCTCGAGTTAGCCAATAAGTTGCTGAGCC | XhoI |
| 57 | LI1153-F | GAGGGATCCGCTAATGTTAGTGGAATCCCTGC | BamHI |
| 58 | LI1153-R | GAGCTCGAGTTATTGTATATTATTTTCATCTGGTTGTAGTG | XhoI |
| 59 | LI0649-F | TCCCATGGCTGAGGCTGTTGAACACTTTG | NcoI |
| 60 | LI0649-R | GGCTCGAGTTAGAATCTATAAGTAGCTCCTACC | XhoI |
| 61 | LI0169-F | CGCCATGGACAGTGATGAGGACCTTAGTACAG | NcoI |
| 62 | LI0169-R | AGCTCGAGTAGGAATCCACCACTGATCAAG | XhoI |
| 63 | LI0786-F | GAGGGATCCATGTTGTTATATATAAATAAAGAACACATTATTG | BamHI |
| 64 | LI0786-R | GAGCTCGAGTTATACTTCTTCTGTATAATAATTTTGTTCA | XhoI |
| 65 | LI1171-F | GAGGGATCCATGTTCAAAAAAATATATGTTTTTTATATCAC | BamHI |
| 66 | LI1171-R | GAGCTCGAGTTATTCATTAGGGACAATAATAGGTGTTAC | XhoI |
| 67 | LI0608-F | GAGGGATCCATGCATCTATATAATACTATGGAAAAG | BamHI |
| 68 | LI0608-R | GAGCTCGAGTTATAAAATATCCCACACCTGACC | XhoI |
| 69 | LI0726-F | GAAGGATCCATGACCATTGAAAAGGGGAGATAC | BamHI |
| 70 | LI0726-R | CGCCTCGAGTTATATTTTTAAAGCTGTTTGTAAATC | XhoI |
| 71 | LI0823-F | GAGGGATCCATGGATATACTACTTCCCTTTGAAAAAAGAC | BamHI |
| 72 | LI0823-R | GAGCTCGAGTTAAAATACTTTTGCACCATCTTCTG | XhoI |
| 73 | LI0625-F | GAGGGATCCATGGCTTCTAAAGAAATCCTTTTTG | BamHI |
| 74 | LI0625-R | GAAGGCGGCCGCTAGTACATACCGTCCATACCACC | NotI |
| 75 | LI0794-F | GAGGAATTCATGGATGATATTTTTAATATGACAGTC | EcoRI |
| 76 | LI0794-R | GAGCTCGAGTTATTCTGTTTTTTCATGCTCTATATCTA | XhoI |

### Expression and Purification of Recombinant Proteins:

Recombinant proteins were expressed in LOBSTR-BL21 (DE3) pRosetta2 E. coli (Kerafast, Inc., Boston, MA, USA) after transformation with plasmid. E. coli grown to mid-exponential phase (OD=0.6) in 2×YT medium plus Kanamycin (50 μg/mL) and induced by the addition of IPTG to 1 mM. Bacteria that express LI1153, LI0710, LI0649 and LI0169 were incubated for 16 hours at 16° C. with shaking at 200 rpm. Bacteria that express LI0786, LI0726, LI0823, LI0794 and LI0625 were incubated for 4 hours at 37° C. with shaking at 200 rpm.

Bacteria transformed with plasmids coding for LI1153, LI0710, LI0649 and LI0169 were harvested by centrifugation and resuspended in urea lysis buffer (8 M urea, 50 mM NaHPO₄, 300 mM NaCl), followed by sonication to lyse bacterial cells. Lysate was centrifuged at 20,000×g for 15 minutes to remove insoluble material. The supernatants containing the recombinant proteins of interest were incubated with 1 mL His60 Superflow Resin (Clontech, Takara Bio USA, Inc., Mountain View, CA, USA) equilibrated with urea lysis buffer and nutated for up to 4 hours. The solution was poured into the Ni Superflow Resin column and the flow through was collected and added to the top of the column twice. The column was then washed with 10 mL of wash buffer 1 (8 M urea and 20 mM imidazole in 250 mM sodium phosphate buffer and 1.5 M NaCl, pH 8.0). Next, the column was washed with wash buffer 2 (8 M urea and 40 mM imidazole in 250 mM sodium phosphate buffer and 1.5 M NaCl, pH 8.0). The proteins were eluted from the Ni-column using 6 mL of elution buffer (8 M urea and 500 mM imidazole in 250 mM sodium phosphate buffer and 1.5 M NaCl, pH 8.0).

Bacteria transformed with plasmids coding for LI0786, LI0726, LI0823, LI0794 and LI0625 were harvested by centrifugation and the pellets were resuspended in approximately 50 mL of buffer consisting of Sigma EDTA-free protease inhibitor tablets dissolved in 100 mL of 10 mM PBS and 1% triton x-100. The solution was subjected to a French Press (Avestin C3 Homogenizer) at 15,000 psi.

For rLI0786, rLI0794 and rLI0625, the lysed bacteria were pelleted to remove bacterial debris and 10 mL aliquots of supernatants containing the recombinant proteins of interest were incubated with 1 mL His60 Superflow Resin (Clontech, Takara Bio USA, Inc., Mountain View, CA, USA) equilibrated with urea lysis buffer and nutated for up to 4 hours. The solution was poured into the Ni Superflow Resin column and the flow through was collected and added to the top of the column twice. The column was then washed with 10 mL of wash buffer 1 (8 M urea and 20 mM imidazole in 250 mM sodium phosphate buffer and 1.5 M NaCl, pH 8.0). Next, the column was washed with wash buffer 2 (8 M urea and 40 mM imidazole in 250 mM sodium phosphate buffer and 1.5 M NaCl, pH 8.0). The proteins were eluted from the Ni-column using 6 mL of elution buffer (8 M urea and 500 mM imidazole in 250 mM sodium phosphate buffer and 1.5 M NaCl, pH 8.0).

For rLI0726 and rLI0823, the lysed bacteria were pelleted and this fraction was harvested to obtain the recombinant proteins. The bacterial pellet was resuspended in 10 mL of Equilibration buffer (50 mM sodium phosphate buffer, 300 mM NaCl, pH 8.0). Then 2 ml of resuspended pellet was then diluted with 20 ml of Equilibration buffer plus 1% Triton x-100. This solution was spun at 12,000×g for 15 minutes. This pellet was resuspended in Wash Buffer 3 (6 M guanidine hydrochloride and 250 mM sodium phosphate buffer, pH 8.0). Ten mLs of the solution were incubated with 1 mL His60 Superflow Resin (Clontech, Takara Bio USA, Inc., Mountain View, CA, USA) equilibrated with urea lysis buffer and nutated for up to 4 hours. The flow through was collected and added to the top of the column twice. Then the resin was washed with Washed buffer 2. The proteins were eluted from the Ni-column using 6 mL of Elution buffer (8 M urea and 300 mM imidazole in 250 mM sodium phosphate buffer and 1.5 M NaCl, pH 8.0).

For dialysis for all recombinant proteins, urea was removed from the purified proteins following a stepwise dialysis with buffers (consisting of 4 M and 2 M Urea in 250 mM sodium phosphate buffer and 1.5 M NaCl, pH 8.0) using 6-8,000 MWCO Spectra/Por® molecular porous membrane tubing (Spectrum®, California, USA). The dialyzed fraction was applied to the top of Amicon 3 kDa centrifugal filters (Millipore/Simga) and they were centrifuged at 3,000 g for up to 1 hour. Phosphate buffered saline (100 mM, approximately 5 mL) was added to the top of the Amicon membrane and centrifugation was repeated to flush the urea from the protein of interest (which remained at the top of the membrane). The protein of interest was resuspended in 2 mL PBS and quantified with BCA assay.

### Protein Expression:

Proteins from the bacteria in the presence and absence of IPTG were subjected to SDS-PAGE analysis (8%-12%) with Coomassie blue staining to show induction of proteins. Western blot analysis showed that rLI0710, rLI1153, rLI0169, rLI0649 and rLI0625 were bound by antibodies from pig sera from animals with clinical symptoms of PHE.

### Animals and Generation of Immune Serum:

Rabbit serum against whole cell L. intracellularis was acquired as reported in Obradovic et al., J. Microbiol. Meth. (2016) 126:60-66. To obtain hyperimmune serum for rLI0710, rLI0649, rLI0625 and rLI1153, four female New Zealand White rabbits (2-3 kg weight) were kept in isolation units. Rabbits were injected via the subcutaneous route with an inoculum consisting of 100 μg of recombinant protein for the first immunization and 50 μg of the same recombinant protein for two booster injections. For all injections, recombinant proteins were resuspended in 500 μL sterile PBS and mixed with 500 μL sterile Incomplete Freund's adjuvant (Sigma-Aldrich) to 1 mL final volume and the vaccines were administered subcutaneously at 4 injection spots with 250 μL of inoculum per site. Each rabbit received one of the four recombinant proteins on day 0, 20, and 40. Rabbit immune sera were collected via exsanguination following euthanasia (Euthanyl, Bimeda-MTC Animal Health INC., Cambridge ON, Canada) 60 days after the first vaccination. All blood samples were collected and centrifuged (2500×g) then sera were stored at −20° C. until use.

Removal of Antibodies Against LPS from Rabbit Immune Serum:

To preclear any LPS-specific antibodies from sera, 10000 EU/mL LPS from E. coli 055:B5 (Sigma-Aldrich) was incubated per mL of each rabbit serum, for one hour at room temperature to allow serum anti-LPS antibodies to bind. After one hour of incubation, endotoxin-removing gel (Pierce High-Capacity Endotoxin removing gel, Thermo Scientific) was used according to manufacturer's protocol, to remove LPS and LPS bound antibodies from rabbit sera. Flow-through fractions before elution of LPS and after elution of LPS were collected and subjected to WB to test the efficacy of the clearing procedure. WB was performed on LPS (as a control), whole cell L. intracellularis, and all 4 recombinant proteins, and detection was performed using LPS-cleared rabbit serum in 1:500 dilution as primary antibody and anti-rabbit IR 800 antibody (1 μg/mL; Li-COR) as secondary antibody.

### Neutralization Assay:

To determine the effect of recombinant L. intracellularis protein-specific sera on penetration of bacteria into IPEC cells, a neutralization assay was performed using carboxyfluorescein succinimidyl ester (CFSE)-stained bacteria, as previously described (Obradovic et al., J. Microbiol. Meth. (2016) 126:60-66). Briefly, CF SE was used to stain avirulent L. intracellularis and stained bacteria were incubated with low (500 μg/mL), medium (1000 μg/mL) and high (2000 μg/mL) complement-inactivated, LPS precleared rabbit hyperimmune serum for 1 hour at room temperature. Bacteria bound with serum antibodies were used to infect 10⁵ IPEC-1 cells in a 24 well plate (Corning) incubated in a tri-gas environment (10% hydrogen, 10% carbon dioxide and 80% nitrogen gas (Praxair Canada Inc., Mississauga, ON, Canada)) in Ziploc™ bags at 37° C. (Vannucci et al., J. Clin. Microbiol. (2012) 50:1070-1072). After 4 hours of incubation, cells were trypsinized then centrifuged at 500×g for 5 minutes to remove medium and unbound bacteria. The cells were then re-suspended in PBS (Gibco Life Technologies) with 2% FBS (Gibco Life Technologies) and analyzed by flow cytometry. This assay was repeated 4 times independently to obtain biological replicates. Flow cytometric analysis was performed using a BD FACS Calibur™ flow cytometer (BD Biosciences, Mississauga, ON, Canada). CFSE fluorescence was detected in the FL1 channel with gating selected based on uninfected IPEC-1 cells (negative control) and IPEC-1 cells infected with CFSE labelled bacteria (positive control). Thirty thousand events were acquired per sample and flow cytometer results were analyzed in Kaluza software (Beckman-Coulter, Indianapolis, Indiana, USA). The percent inhibition was calculated using the following formula: Percent inhibition=(1−% of fluorescence of CFSE bacteria incubated with serum/% of fluorescence of CF SE bacteria (control))×100.

Vaccine trials to assess immunogic properties of recombinant L. intracellularis proteins: Animal ethics: All experimental procedures were conducted in accordance with the guidelines of the Canadian Council on Animal Care (CCAC) under approval from the Animal Research Ethics Board at the University of Saskatchewan. Pigs were all Landrace/Large White from Prairie Swine Centre, Inc. (PSC, Saskatoon, Saskatchewan).

### Animal Trials and Sample Collection:

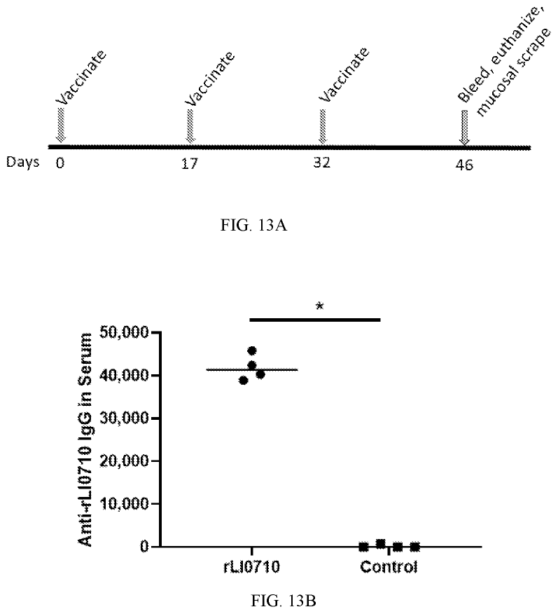

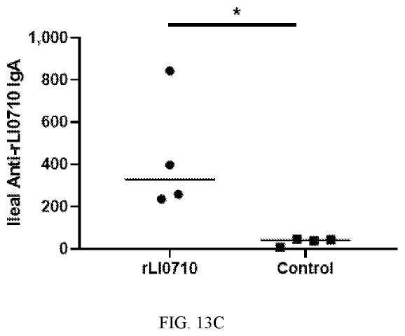

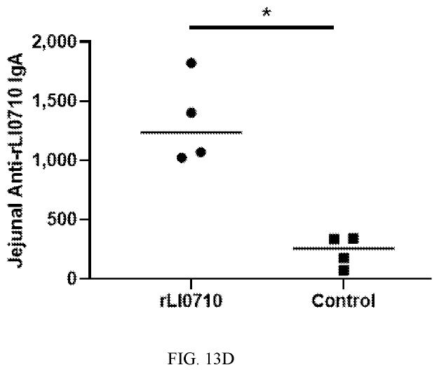

Trial 1: FIG. 13A shows the schematic of the immunization trial. Parenteral vaccination of weaner piglets with vaccine consisting of rLI0710 and formulated with VIDO Triple adjuvant. Weaner piglets (5 weeks of age, n=4) were immunized by the intramuscular route with 300 μg rLI0710 formulated with 300 μg poly IC, 600 μg host defense peptide, and 300 μg polyphosphazene (VIDO Triple Adjuvant). They received booster doses 17 days later and 32 days later. Control animals were immunized by the same route and on the same days with VIDO triple adjuvant (n=4). Pigs were euthanized on day 46. Serum IgG titres were quantified (FIG. 13B). The ileum and jejunum were scraped and mucosal anti-rLI0710 IgA was quantified (FIG. 13C-D).

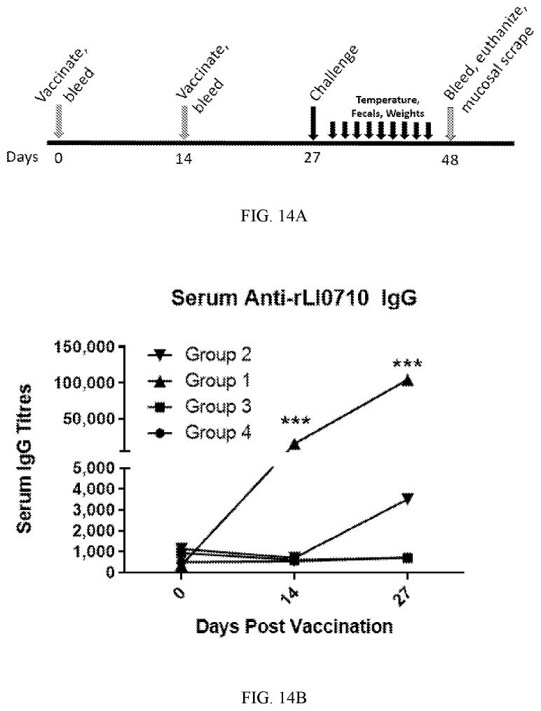

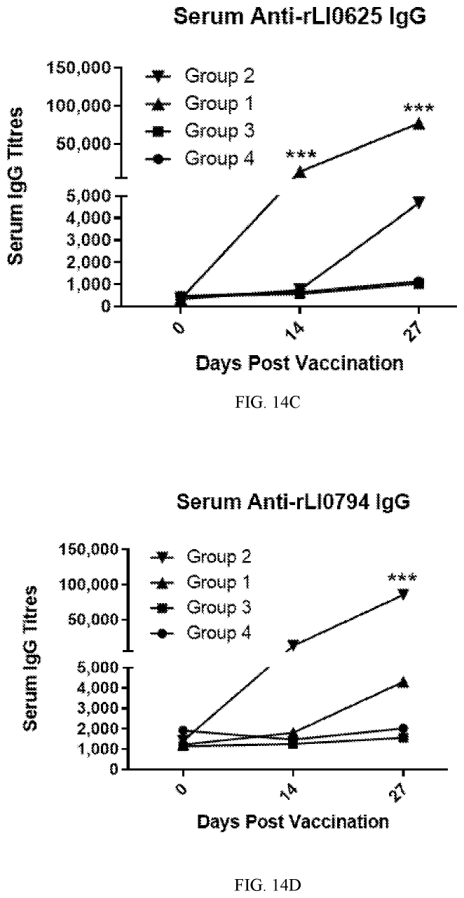

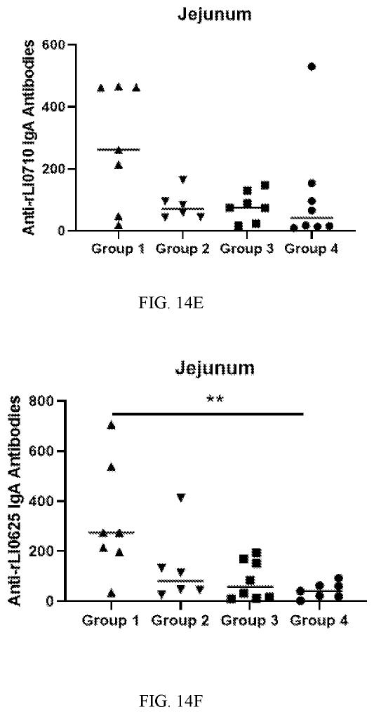

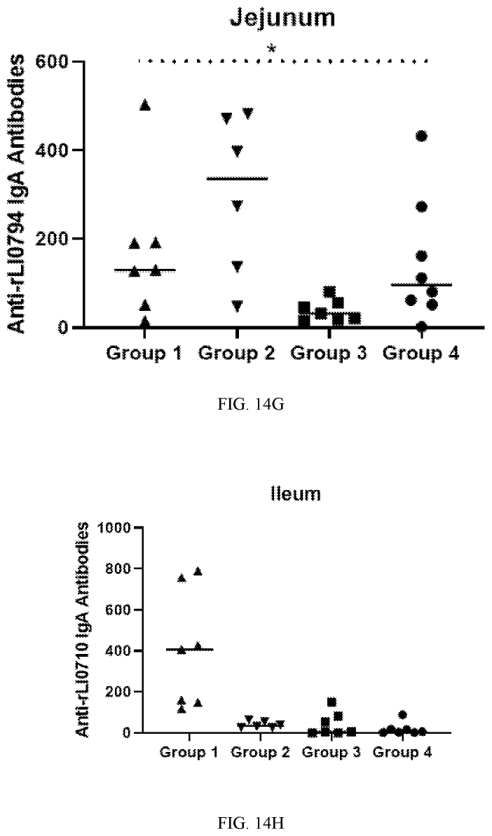

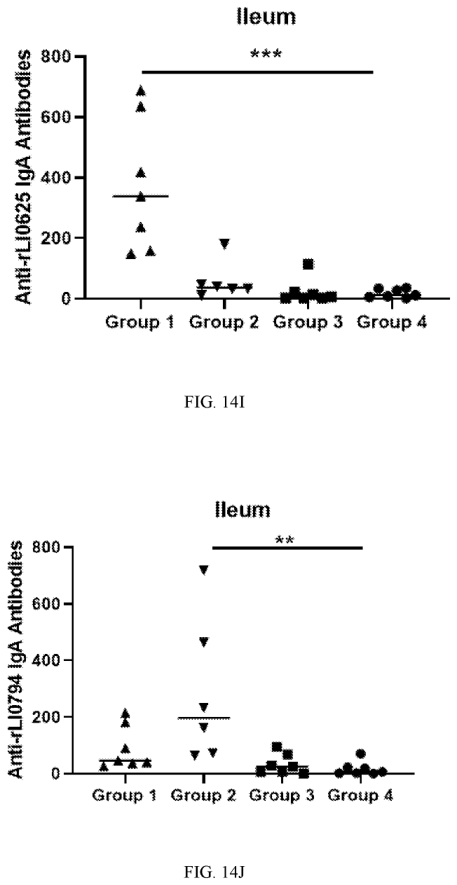

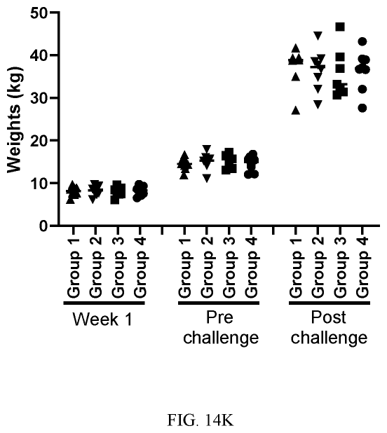

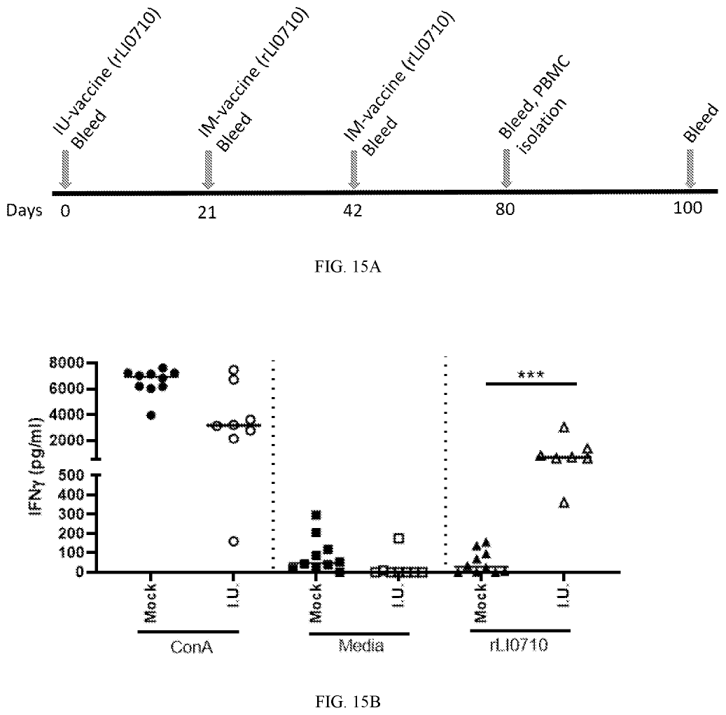

Trial 2: Parenteral vaccination with vaccines consisting of 3 recombinant antigens and formulated with EMULSIGEN® adjuvant (MVP Laboratories, Inc., Omaha, NE, USA). Weaner piglets (5 weeks of age) were immunized by the intramuscular route with 50 μg rLI0710, 50 μg rLI0169 and 50 μg rLI0625 formulated with 30% EMULSIGEN® (700 μL for all antigens: 700 μL EMULSIGEN®, for a 1.4 mL total volume) (Group 1, n=8); 50 μg rLI0794, 25 μg rLI0786 and 50 μg rLI0726 formulated with 30% EMULSIGEN® (1:1 volume with 1.4 mL total volume) (Group 2, n=8); weaners immunized with EMULSIGEN® alone (Group 3, challenged control group, n=7) and weaners immunized with EMULSIGEN® alone (Group 4, non-challenged control group, n=7). Weaner piglets received one booster dose 14 days later (FIG. 14A). Piglets from Groups 1-3 were then fasted overnight and then challenged orally (gavaged) on day 27 with approximately 1.9×10⁸ pathogenic L. intracellularis in 40 mL. Serum antibodies were assessed (FIGS. 14B-D) and antibody titres from mucosal scrapings at time of euthanization were also assessed (FIGS. 14E-J). Rectal temperatures were collected and piglets were weighed up to and including carcass weights (FIG. 14K). Fecal samples were collected for 18 days and assessed for shedding of L. intracellularis using PCR analysis (Table 4). Table 4 shows the fecal samples from challenged and control pigs subjected to PCR analysis to identify L. intracellularis DNA as evidence of bacterial shedding. Fecal L. intracellularis calculated from 200 mg feces per pig per time point (processed to 200 μL volume) are shown. Each symbol represents an individual animal and the horizontal bars show the median value of each column. Each box represents one animal and one time point. The key for the concentration of L. intracellularis genomic DNA per 200 mg feces is: +/− (<200), + (200-1,000), ++ (1,000-5,000), and +++ (>5,000).

TABLE 4
PCR analysis of fecal samples from challenged and control pigs

Group 1 (rLI0710, LI0625, rLI0169)

| | | | | | | | |
|---|---|---|---|---|---|---|---|
| Day 8 | +/− | +/− | +/− | +/− | +/− | +/− | +/− |
| Day 11 | +/− | +/− | +/− | +/− | +/− | +/− | +/− |
| Day 13 | +/− | ++ | + | +/− | + | + | +/− |
| Day 15 | + | ++ | + | + | + | + | +/− |
| Day 18 | ++ | ++ | + | ++ | ++ | + | + |

| | Group 2 (rLI0786, rLI0726, rLI0794) | | | | | | | |
|---|---|---|---|---|---|---|---|---|
| Day 8 | +/− | +/− | +/− | +/− | +/− | +/− | +/− | +/− |
| Day 11 | +/− | + | + | +/− | +/− | +/− | +/− | +/− |
| Day 13 | + | ++ | ++ | + | +/− | + | +/− | +/− |
| Day 15 | + | ++ | +++ | + | + | + | + | +/− |
| Day 18 | ++ | +++ | ++ | ++ | ++ | ++ | + | ++ |

| | Group 3 (Challenged, Unvaccinated) | | | | | | |
|---|---|---|---|---|---|---|---|
| Day 8 | +/− | +/− | +/− | +/− | +/− | +/− | +/− |
| Day 11 | +/− | + | +/− | +/− | + | + | + |
| Day 13 | +/− | + | + | + | + | ++ | ++ |
| Day 15 | + | ++ | + | + | + | ++ | ++ |
| Day 18 | +++ | +++ | ++ | ++ | +++ | +++ | +++ |

Group 4 (Unchallenged, Unvaccinated)

| | | | | | | | | |
|---|---|---|---|---|---|---|---|---|
| Day 8 | +/− | +/− | +/− | +/− | +/− | +/− | +/− | +/− |
| Day 11 | +/− | +/− | +/− | +/− | +/− | +/− | +/− | +/− |
| Day 13 | +/− | +/− | +/− | +/− | +/− | +/− | +/− | +/− |
| Day 15 | +/− | +/− | +/− | +/− | +/− | +/− | +/− | +/− |
| Day 18 | +/− | +/− | +/− | +/− | +/− | +/− | +/− | +/− |

Trial 3: Mucosal vaccination with proteins formulated with VIDO Triple Adjuvant. FIG. 15A shows the schematic of the immunization trial. Gilts in estrus were immunized at breeding such that the vaccines were added to 80 mL commercial extended semen (PIC Genetics) which had been subjected to repeated −80° C. to 37° C. temperature changes to kill the semen (although the sperm did not appear ruptured under the microscope). The vaccine for the first dose consisted of 400 μg rLI0710 formulated in 1 mL total volume with 1200 μg poly IC, 2400 μg host defense peptide 1002, and 1200 μg polyphosphazene (n=8). The semen plus vaccine was administered to a catheter tube following normal intracervical breeding practices for gilts in estrus. Thus, this vaccine was directed to the uterus. Approximately 21 days later, the gilts returned to estrus and were mock-bred a second time with killed semen plus VIDO triple adjuvant. Gilts were also immunized with rLI0710 (400 μg) formulated with 400 μg poly IC, 800 μg host defense peptide 1002, and 400 μg polyphosphazene VIDO triple adjuvant and this vaccine was administered via the intramuscular route for vaccinated animals (i.e. not in mock-immunized pigs). For the third vaccination, gilts were immunized exactly as stated with the second dose except live semen was used. Control animals were bred following normal husbandry practices with live semen at estrus (Mock, n=10) without intrauterine or intramuscular VIDO Triple Adjuvant. For all animals, approximately 38 days later, peripheral blood mononuclear cells (PBMCs) were obtained from all gilts and the cells were tested for ex vivo IFNγ production response to antigens (FIG. 15B).

### PBMC Isolation and Cell Ex Vivo Restimulation:

PBMCs were isolated from blood collected using EDTA Vacutainers (BD Biosciences) then centrifuged at 1100×g for 30 min. The buffy coats were collected and layered onto Ficoll-Paque plus (GE life sciences) and centrifuged at 400×g for 40 min. The PBMC layer was collected, washed in PBS 3 times with centrifugation at 250×g for 10 minutes. Cells were plated at a total of 1×10⁶ cells per well. Cells were cultured for 24 hours with Con A (5 μg/mL), rLI0710 (2 μg/mL) or media. The plates were subjected to centrifugation at 500×g for 10 min and the supernatant was removed and frozen at −20° C. for later IFNγ quantification.

### IFNγ ELISA:

Plates were coated overnight with mouse anti recombinant porcine interferon γ (Fisher ENMP700 (Pierce Endogen)) in 1.0 μg/ml coating buffer. Plates were washed 4× with TBST. Samples were applied diluted 1:4 in diluent (TBST+0.5% skim milk). Standard rPoIFNγ (Ceiba Geigy 212243 lot #016144) is prediluted to 4000 μg/mL in diluent. The standard starts from 4000 μg/mL and two fold dilutions are done. Plates were incubated overnight at 4° C. Plates were washed 4× with TBST and 100 μL of detection antibody (rabbit anti recombinant porcine IFNγ (Fisher ENPP700 (Pierce Endogen)) diluted to 2 μg/mL was added to each well for 1 hour incubation at room temperature. Plates were washed 4× with TBST and goat anti Rabbit IgG (H+L) biotin (Zymed #62-6140) (1/10,000) was incubated to 100 uL per well for 1 hour at room temperature. Plates were washed with 4×TBST then streptavidin alkaline phosphatase (Jackson #016-050-084; 50% glycerol) diluted 1/5000 in diluent was added to each well for 1 hour at room temperature. Finally, plates were washed 4× with TBST then 100 μL of PNPP substrate (diluted in PNPP buffer to 1 mg/mL) was added to each well for approximately 30 minutes at room temperature. The reaction was stopped by the addition of 30 uL of 0.3M EDTA and the plates were read at λ405 nm, reference λ490 nm. The IFNγ concentration of the samples was determined from the standard curve.

### Antibody ELISAs:

Antibody ELISAs were performed on serum and mucosal scrapings of jejunum and ileum to measure antibody response the antigens rLI0710, rLI0625, and rLI0794. Immulon II plates (VWR) were coated over night with up to 2 μg/mL protein in coating buffer. Plates were washed with tris-buffered saline with 2% Tween-20 (TBST). Sera were serially diluted in assay diluent buffer TBST. After 2 hours incubation, the plates were washed in TBST then incubated for 1 hour with 1/5000 Alkaline phosphatase-conjugated Goat anti-Pig IgG (H+L) (KPL catalogue #151-14-06). ELISAs were then developed with 1 mg/mL p-nitrophenyl phosphate in DE buffer (1 M diethanolamine, 0.5 M magnesium chloride) and absorbance at λ405 nm was measured on a SpectraMax plus microplate reader (Molecular Devices). All end-point titers were determined using 4 fold serial dilutions with initial dilutions of serum and culture supernatants performed at 1:4.

### PCR Analysis:

Fecal samples (200 mg) were collected from all piglets in all groups every second day starting on the day of challenge and ending after 18 days. The samples were processed using QIAmp DNA Stool Kit (Qiagen) as reported by the manufacturer. Primers designed against amino acid ABC transporter substrate-binding protein (GlnH, Locus LI0754) from the L. intracellularis PHE/MN1-00 genome (Forward: 5′-GGTTAGTCGTTGCCCATGATA-3′ (SEQ ID NO: 77), Reverse: 5′-CTGCGATATGCTCCCATAGTT-3′ (SEQ ID NO: 78)) were used to quantify genome copy number. Quantitative real time PCR (qPCR) was conducted using Kapa Syber Green Mastermix (Kapa Biosystems, Wilmington, MA) with data collected using a Step One Real-Time PCR System (Applied Biosystems by Life Technologies).

### Statistical Analysis:

The Shapiro-Wilk normality test was used to determine whether data follows a Gaussian distribution. A one way ordinary ANOVA test was used to compare means of values of percentage of inhibition of each serum. All statistical analyses and graphing were performed using GraphPad Prism 5 software (GraphPad Software, San Diego, CA). Differences were considered significant if p was less than 0.05.

### Example 1

### Identification of Bacterial Proteins that Interacted with IPEC Cells

2DE coupled with WB analysis and MS/MS was utilized to identify L. intracellularis proteins that interacted with IPEC-1 cell surface proteins and recognized by rabbit hyperimmune serum. On the WB, Cy5-labeled L. intracellularis proteins appeared as red spots whereas the green/yellow spots indicated that these proteins were also bound by rabbit antibodies and therefore immunogenic. Characteristic accumulation of the abundant albumin protein was observed in the region from 75 kDa to 100 kDa which was also confirmed by MS/MS analysis. Despite 3 washes with serum-free medium, contaminating albumin was consistently present and attempts to preclear samples of albumin failed. However, despite the presence of albumin, low MW proteins were well separated and both red and green spots were visualized, indicating Cy5-labeled L. intracellularis proteins alone and bound by antibodies. Using WB as a template, the corresponding spots were isolated in the silver-stained preparative gel. These proteins/spots were excised from the gel and subjected to MS/MS analysis.

Bioinformatics analysis of MS/MS detected proteins by UNIPROT and PFAM revealed 11 unique bacterial proteins identified by MS (Table 2). Four of the indicated proteins were predicted to be expressed in the outer membrane of bacteria and these were selected for further analysis. These proteins were identified as Flagellin (FliC, LI0710), Putative outer protein N (YopN, LI1153), ABC dipeptide transport system (OppA, LI0169) and autotransporter (LI0649) (also known as LatA; Watson et al., Clin. and Vaccine Immunol. (2011) 18:1282-1287).

Flagellin (LI0710, FliC; SEQ ID NOs: 1 and 2; NCBI-proteinID: CAJ54764; UNIPROT: Q1MQG3, MW 31 kDa, PI 5.97 ExPasy) is a subunit protein that polymerizes to form flagella and plays an important role in bacteria locomotion and chemotaxis. Flagellum has been observed as a bacterial cell structure oft intracellularis (Lawson et al., J. Compar. Path. (2000) 122:77-100). Flagellin LI0710 has 293 amino acids and consists of a PF00669 (PFAM) domain between amino acids 5 to 141 and a PF00700 (PFAM) domain between amino acids 208-291. Flagellin is a TLR5 agonist which plays an important role in the process of immune recognition of Gram negative bacteria. Due to its dual antigen and adjuvant nature, L. intracellularis Flagellin LI0710 is ideal for use in a subunit vaccine.

LI1153, annotated as Putative outer protein N (SEQ ID NOs: 10 and 11; NCBI-proteinID: CAJ55207; UNIPROT: Q1MP70; MW 44 kDa; PI 4.62, ExPasy) is a part of the T3 SS system. LI1153 consists of two prominent domains with important functions during invasion into eukaryotic cells: HrpJ positioned between 63-222 amino acids, PF07201 (PFAM), and TyeA positioned between 299-378 amino acids, PF09059 (PFAM). The HrpJ domain is predicted to be part of the T3SS and related proteins include SsaL and InvE invasion protein from Salmonella typhimurium which are involved in host pathogen interaction (Michael et al., Molec. Microbiol. (1997) 24:155-167) and invasion (Ginocchio et al., Proc. Natl. Acad. Sci. USA (1992) 89:5976-5980), respectively. A related E. coli protein, SepL, plays a crucial role in the infection of enterohemorrhagic E. coli and has a potential role in secretion of EspA, EspD, and EspB (Kresse et al., J. Bacteriol. (2000) 182:6490-6498). Domain TyeA, identified in Yersinia spp., is located on the bacterial surface, plays an important role in controlling the secretion of effector proteins, and contributes to a translocation-control apparatus within the T3 SS (Iriarte et al., EMBO J. (1998) 17:1907-1918). These secretion regulator proteins have been described as “gate-keepers” in major Gram negative bacterial species and their deletion leads to decreased secretion of translocon proteins or increased secretion of effector proteins (Burkinshaw et al., Mol. Cell Res. (2014) 1843:1649-1663). Based on a comparison of the structure of other T3SS systems (See, e.g., Alberdi et al., Vet. Microbiol. (2009) 139:298-303), it appears that LI1153, annotated as Putative Outer protein N, corresponds to the predicted second part of the L. intracellularis T3 SS and has a role in controlling secretion of effector proteins into host cells. Given the interaction between this protein and target cells as described herein, the protein likely plays a role in invasion and attachment of the bacteria to small intestine enterocytes.

LI0169, oppA (SEQ ID NOs: 7 and 8); NCBI-proteinID: CAJ54225; UNIPROT: Q1MS01; MW 63.5 kDa and PI 6.59, ExPasy) is coded by gene oppA and predicted to be expressed on the bacterial membrane as part of the ATP-binding cassette (ABC) transporter complex. In bacteria, the ABC transporter complex plays a central role in the uptake of sugars, amino acids, metals, growth factors, ions and other solutes across the cell membrane (Singh et al., Microbiol. (2008) 154:797-809). LI0169 consists of a transmembrane helical domain (12-34 aa), a periplasmic domain (98-456 aa, PF00496) and an ATP-binding coiled domain (476-496 aa) at the intracellular face of the membrane that together form a central pore. It transports di- and tripeptides in an ATP-dependent manner (Higgins et al., Microbiol. (2001) 152:205-210).

Protein LI0649 (SEQ ID NOs: 4 and 5; NCBI-protein ID: CAJ54703; UNIPROT: Q1MQM4; PI 4.81, MW 91.9 kDa; ExPasy) has been identified previously as autotransporter protein LatA (Watson et al., Clin. and Vaccine Immunol. (2011) 18:1282-1287). As shown herein, LatA is immunogenic as it was bound by rabbit anti-L. intracellularis hyperimmune serum and has a role in bacterial-host interactions. LatA had a predicted molecular mass (ExPasy) of 91.9 kDa but the corresponding protein was 60 kDa using 2DE SDS-PAGE gel, indicating that some cleavage may have occurred. This protein is immunogenic and may be used in a subunit vaccine formulation.

Protein LI0786 (SEQ ID NOs: 13 and 14; NCBI-protein ID: CAJ54840.1; UNIPROT: Q1MQ87; PI 4.70, MW 43.62 kDa; ExPasy) has been predicted to have a DNA polymerase sliding clamp subunit (PCNA homolog). Gene Ontology (GO) indicates that it has 3′-5′ exonuclease function, DNA binding function and DNA-directed DNA polymerase activity.

Protein LI1171 (SEQ ID NOs: 16 and 17; NCBI-protein ID: CAJ55225.1; UNIPROT: Q1MP52, PI 5.71, MW, 62.29 kDa; ExPasy) has the submitted name 5′-nucleotidase/2′,3′-cyclic phosphodiesterase and related esterases. GO Molecular function predicts that it has hydrolase activity, as well as metal ion binding and nucleotide binding activity. The domain for metal binding resides between amino acids 30-250 and the domain for nucleotide binding resides between amino acids 365-518.

Protein LI0608 (SEQ ID NOs: 19 and 20; NCBI-protein ID: CAJ54662; UNIPROT: Q1MQR5, PI 5.55, MW, 55.49; ExPasy) is a Cysteinyl-tRNA synthetase which binds 1 zinc ion per subunit. GO Molecular function predicts that it has an ATP binding site, cysteine-tRNA ligase activity, and zinc ion binding activity. This is some evidence that a homolog in Rickettsia has also been identified as an immunoreactive protein.

Protein LI0726 (SEQ ID NOs: 22 and 23; NCBI-protein ID: CAJ54780.1; UNIPROT: Q1MQE7, PI 5.41, MW, 44.4; ExPasy) is a S-adenosylmethionine synthetase. GO Molecular function predicts that it has ATP and magnesium binding sites and methionine adenosyltransferase activity. We found no evidence that this protein has been used as a vaccine antigen. However, one study showed that S-adenosylmethionine synthetase was differentially expressed between Trypanosoma brucei subspecies and that this homolog may be a potential vaccine target for the development of vaccines to block the transmission of trypanosomes.

Protein LI0823 (SEQ ID NOs: 25 and 26; NCBI-protein ID: CAJ54877.1; UNIPROT: Q1MQ50, PI 6.52, MW, 63.6; ExPasy) is a Xaa-Pro aminopeptidase. It has a Creatinase domain and a Peptidase domain. We found no evidence that this protein has been used as a vaccine antigen.

Protein LI0625 (SEQ ID NOs: 28 and 29; NCBI-protein ID: CAJ54679.1; UNIPROT: Q1MQP8, PI 5.30, MW58.6; ExPasy) is a chaperonin also known as groEL and Cpn60. It plays a role in protein refolding and it has an ATP binding and an unfolding protein binding domain. GroEL is conserved in many pathogenic microbes and has been identified as immunogenic and/or investigated as a candidate for vaccine development against many pathogens including Edwardsiella tarda (Liu et al 2016), Leptospira interrogans (Natarajaseenivasan et al 2011), Bordetella pertussis (Luu et al. 2020), etc. We found no evidence that this protein has been identified as immunogenic in L. intracellularis.

Protein LI0794 (SEQ ID NOs: 31 and 32; NCBI-protein ID: CAJ54848.1; UNIPROT: Q1MQ79, PI 4.73, MW, 23.5 ExPasy) is an ATP-dependent Clp protease subunit (ClpP). It has chymotrypsin-like activity and it plays a major role in the degradation of misfolded proteins. When 14 ClpP subunits assemble into 2 heptameric rings, they form a disk-like structure with a central cavity that resembles a eukaryotic proteasome. Alteration of ClpP function has been shown to impact pathogen virulen and infectivity which suggests that it may be an attractive target for antimicrobials (Moreno-Cinos, et al 2019). ClpP has been investigated as a potential antigen for Streptococcus pneumonia vaccines in mice (Wu et al 2010), Cao, et al 2009). We found no evidence that this protein has been identified as immunogenic in L. intracellularis.

### Example 2

### Evaluation of Recombinant Protein Antigenic Properties

Recombinant proteins were expressed using LOBSTR-BL21 (DE3) pRosetta2 E. coli. SDS-PAGE analysis and Coomassie staining confirmed that rLI1153 (44 kDa), rLI0710 (32 kDa), rLI0649 (92 kDa), rLI0169 (64 kDa), rLI0786 (45 kDa), rLI0726 (44 kDa), rLI0794 (25 kDa) and rLI0625 (60 kDa) were expressed at their predicted molecular weights.

Next, to confirm that these recombinant proteins were immunogenic in pigs, serum from pigs diagnosed with PE from an L. intracellularis endemic farm was pooled and used in WB analysis. The sequences of the recombinant proteins used in WB are listed in FIGS. 1C, 2C, 3C, 4C and 10C.

rLI0710, rLI0649, rLI0169, rLI1153 and rLI0625 were recognized by sera from PE-infected pigs, which indicated that these recombinant proteins remained immunogenic and demonstrated the relevance of using rabbit serum to detect antigens of causative agent of porcine proliferative enteropathy. rLI0649 was weakly recognized by sera from PE-infected pigs in the WB possibly due to the fact that the pooled porcine sera were pooled from only 5 animals infected from one farm. To test the immunogenic potential of each of the 4 recombinant proteins, rabbits were vaccinated with one of the four recombinant proteins to generate hyperimmune serum specific for each target. Hyperimmune serum was then used in WB analysis and visualized with anti-rabbit secondary IR800. Recombinant LI1153 was bound by rabbit hyperimmune sera (from a rabbit vaccinated against recombinant LI1153). Recombinant LI0710 (32 kDa) was bound by rabbit hyperimmune sera (from a rabbit vaccinated against recombinant Flagellin). Recombinant LI0649 (92 kDa) was bound by rabbit hyperimmune sera (from a rabbit vaccinated against recombinant LI0649). Finally, Recombinant LI0169 (64 kDa) was bound by rabbit hyperimmune sera (from a rabbit vaccinated against recombinant LI0169). Serum from unimmunized rabbits did not recognize any of the four recombinant proteins. These results indicated that the proteins possessed immunogenic properties.

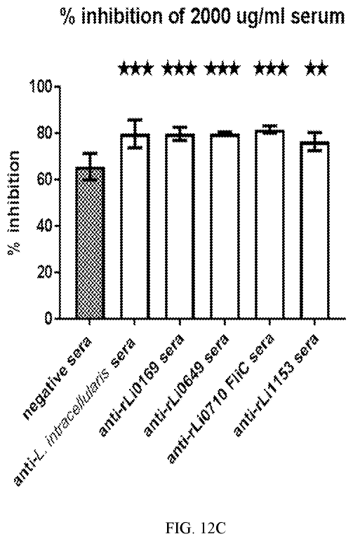

In order to quantify the level of inhibition that antigen-specific antibodies had on preventing penetration of CF SE-labelled avirulent L. intracellularis into eukaryotic cells, neutralization assays were performed as described above (See, FIG. 12). The following sera were tested at low (500 μg/mL), medium (1000 μg/mL) and high (2000 μg/mL) concentrations: rabbit sera before immunization, rabbit sera against whole avirulent bacteria, rabbit hyperimmune serum specific for rLI0169, rLI0649, rLI0710 FliC, or rLI1153. The multiplicity of infection (MOI) of 0.1 of CF SE stained L. intracellularis remained constant. Because others have shown that negative mouse serum showed 48% to 59% inhibition of L. intracellularis invasion, likely due to the presence of anti-LPS antibodies present prior to generation of hyperimmune serum (McOrist et al., Vet. Microbiol. (1997) 54:385-392), negative rabbit serum was tested to determine if it bound to LPS. Rabbit negative serum was found to bind to one band with a molecular weight between 20 and 25 kDa which corresponded to the molecular weight of LPS (Kroll et al., Clin, Diagn. Lab. Immunol. (2005) 12:693-699). Therefore, anti-LPS antibodies were precleared from all sera prior to performing the neutralization assays.

The gate in flow cytometry analysis was based on percentages of fluorescence detected in FL-1 channel for IPEC-1 cells alone and IPEC-1 cells infected with CFSE L. intracellularis. As expected, IPEC-1 cells alone had negligible positive fluorescence events (mean value of 0.20% fluorescence in FL1 channel) and IPEC-1 cells infected with CFSE-labelled L. intracellularis showed a mean value of 8.86% fluorescence in the FL1 channel 4 hours post-infection. The percentage of positive events in the FL1 channel when CFSE-labelled L. intracellularis was incubated with 2000 μg/mL of serum from rabbit immunized with whole bacteria was reduced to 2.29%. FIGS. 12A-12C show the percent inhibition that serum antibodies blocked L. intracellularis invasion of IPEC-1 cells. Negative control sera consisted of pooled sera from rabbits prior to immunization for hyperimmune serum generation. Pre-incubation of CFSE-L. intracellularis with low (500 μg/mL), medium (1000 μg/mL) and high (2000 μg/mL) concentrations of negative control sera showed 46.7% (±7.9), 58.9% (±8), and 65.7% (±5.7) percent inhibition, respectively. This inhibition by negative control serum is not unexpected as others have shown that rabbit polyclonal sera prepared against E. coli and other negative control serum obtained prior to generation of L. intracellularis hyperimmune sera inhibited L. intracellularis penetration of cultured rat enterocytes (IEC-18) (McOrist et al., Vet. Microbiol. (1997) 54:385-392).

Rabbit hyperimmune serum generated against whole L. intracellularis was used as the positive control serum. CF SE-labeled L. intracellularis incubated with low (50011 g/mL), medium (1000 μg/mL) and high (2000 μg/mL) serum resulted in 64.8%±5.7, 73.4%±4.7 and 79.88%±5.9 inhibition of infection, respectively (FIGS. 12A-12C). Relative to the negative control sera, positive control sera inhibited significantly more cellular adhesion/penetration for low (p<0.05; FIG. 12A), medium (p<0.01; FIG. 12B) and high (p<0.001; FIG. 12C) sera concentrations, indicating that anti-L. intracellularis antibodies were neutralizing. To discern whether antibodies specific for rLI0169, rLI0649, rLI0710 and rLI153 blocked bacterial adherence/penetration into IPEC-1 cells, the CF SE-L. intracellularis was pre-incubated with 50011 g/mL (FIG. 12A), 1000 μg/mL (FIG. 12B) and 2000 μg/mL (FIG. 12C) hyperimmune sera specific for each recombinant protein. At the lowest concentration of hyperimmune sera (FIG. 12A), anti-rLI0169 showed 68.7%±5.9 percent inhibition, anti-rLI0649 showed 64%±9.0 percent inhibition, anti-rLI0710/FliC showed 69.5%±5.2 percent inhibition and anti-rLI1153 showed 60.4%±11.8 percent inhibition. With the exception of anti-rLI1153, all 3 hyperimmune sera showed significantly higher percent inhibition relative to the negative control serum (p<0.01, p<0.05, p<0.01, respectively). When the medium and high concentration of each antisera was used, anti-rLI0169 (p<0.01 FIG. 12B, p<0,001 FIG. 12C), anti-rLI649 (p<0.01 FIG. 12B, p<0.001 FIG. 12C), anti-rLI0710/FliC (p<0.01 FIG. 12B, p<0.001 FIG. 12C), and anti-rLI1153 (p<0.01 FIG. 12B, p<0.01 FIG. 12C), all relative to the relative dose of control sera.

Therefore, sera antibodies specific for the recombinant proteins showed comparable inhibitory effect to that observed with the positive rabbit serum against whole bacteria and the inhibitory effect of all sera increased with increased serum concentration. The results from the recombinant serum neutralization assay confirm that use of the recombinant proteins as antigens in subunit vaccine formulations may generate neutralizing antibodies capable of inhibiting L. intracellularis penetration and infection.

Because L. intracellularis is an obligate intracellular bacterium, the cellular immune response is predicted to play the major role in protection against virulent bacteria (Cordes et al., Vet. Res. (2012) 43:9; Guedes et al., Canadian J. Vet. Res. (2010) 74:97-101), however, the humoral immune response may also play an important role in protecting against L. intracellularis infection. IgG antibodies against intracellular bacteria could bridge humoral and cellular immunity by targeting intracellular pathogens to lysosomes through Ab-FcR-mediated stimulation of the host cells (Armstrong et al., J. Exper. Med. (1975) 142:1-16), protection against intracellular bacteria by Fc receptor-mediated lysosomal targeting (Joller et al., Proc. Natl. Acad. Sci. USA (2010) 107:20441-20446) and modulation of cytokine secretion (Polat et al., Immunol. (1993) 80:287-292). Additionally, IgA antibodies play an important role in protection against enteric pathogens. Accumulation of IgA bound to L. intracellularis inside enterocytes and lamina propria has been reported (McOrist et al., Infect. Immun. (1992) 60:4184-4191) and L. intracellularis-specific IgA were detected in intestinal lavage of pigs 3 weeks after experimental infection (Guedes et al., Canadian J. Vet. Res. (2010) 74:97-101). Results from a vaccine trial where animals were vaccinated orally and challenged with virulent L. intracellularis indicated that protection was associated with mucosal cytokine and specific IgG and IgA responses and that systemic antibody responses were boosted following challenge (Nogueira et al., Vet. Microbiol. (2013) 164:131-138.

Based on the foregoing, a subunit vaccine comprised of one or more of the identified L. intracellularis proteins is predicted to induce a specific protective immune response against L. intracellularis in the intestinal mucosa of pigs.

Thus, immunogenic compositions and methods of making and using the same for treating and preventing L. intracellularis infection using L. intracellularis recombinant antigens are described. Although preferred embodiments of the subject invention have been described in some detail, it is understood that obvious variations can be made without departing from the spirit and the scope of the invention as defined by the appended claims.

Trial 1: Verifying that rLI0710 Lawsonia Intracellularis Protein is Immunogenic in Pigs.

Weaner piglets (5 weeks of age, n=4) were immunized by the intramuscular route with 300 μg rLI0710 formulated with VIDO Triple Adjuvant. They received booster doses 17 days later and 32 days later. Control animals were immunized by the same route and on the same days with VIDO Triple Adjuvant (n=4). Piglets were euthanized on day 46. Serum was collected to assess anti-rLI0710 IgG (FIG. 13B). Ileum (FIG. 13C) and jejunum (FIG. 13D) were scraped to allow mucosal anti-rLI0710 IgA quantification. Serum anti-rLI0710 IgG titres were significantly higher (p<0.05) in vaccinated versus mock-vaccinated animals (FIG. 13B). Ileal (FIG. 13C) and jejunal (FIG. 13D) anti-rLI0710 IgA titres were significantly higher (p<0.05, p<0.05, respectively) in vaccinated versus mock-vaccinated animals. These data suggest that recombinant rLI0710 formulated with VIDO Triple Adjuvant and administered to piglets via the intramuscular route triggered significant systemic and intestinal antibodies.

Trial 2: Verifying that Intramuscular Vaccines Comprised of Immunogenic Recombinant L. intracellularis Proteins are Immunogenic and Protective Against Infectious Challenge.

Weaner piglets (5 weeks of age) were immunized by the intramuscular route with 50 μg rLI0710, 50 μg rLI0169 and 50 μg rLI0625 formulated with 30% EMULSIGEN (1:1; 700 μL for all antigens: 700 μL EMULSIGEN® for a 1400 μL total volume) (Group 1, n=8). Group 2 animals (n=8) were immunized by the same route with 50 μg rLI0794, 25 μg rLI0786 and 50 μg rLI0726 formulated with 30% EMULSIGEN (1:1 volume with 1400 μL total volume). Group 3 weaners (challenged control group, n=7) and Group 4 weaners (non-challenged control group, n=7) were immunized with EMULSIGEN alone. All animals received one booster dose 14 days later.

Group 1 showed significantly higher anti-rLI0710 (FIG. 14B) and anti-rLI0625 (FIG. 14C) IgG titres after 14 (p<0.001) and 27 (p<0.001) days, respectively, relative to Group 4. Animals in Group 2 showed significantly higher anti-rLI0794 IgG after 27 days (p<0.001) relative to Group 4 (FIG. 14D). These data suggest that rLI0710, rLI0625 and rLI0794 antigens triggered significant humoral immunity when formulated with EMULISGEN in vaccines administered by the intramuscular route. We also observed that all piglets had low level antibodies specific for recombinant L. intracellularis antigens at Day 0 suggesting prior exposure (of the piglets or from colostrum produced by their dams) to L. intracellularis. This prior exposure is expected as these bacteria are endemic and the data suggested that a humoral response to the subunit vaccine is observed despite prior exposure.

Mucosal scrapings on the jejunum and ileum on day 48 were also investigated for anti-rLI0710, anti-rLI0625 and anti-rLI0794 IgA titres. Mucosal tissues did not show elevated anti-rLI0710 IgA in jejunum (FIG. 14E) or ileum (FIG. 14H) in any group relative to Group 4. Mucosal tissues from pigs immunized with rLI0710, rLI0625 and rLI0169 (Group 1) showed significantly elevated anti-rLI0625 IgA in jejunum (FIG. 14F, p<0.01) and ileum (FIG. 14I, p<0.001) relative to Group 4. Finally, mucosal tissues showed significantly different anti-rLI0794 IgA in the jejunum (FIG. 14G, p<0.05) across all groups but not in any specific group relative to Group 4. However, pigs immunized with rLI0794, rLI0726 and rLI0786 plus VIDO Triple Adjuvant (Group 2) showed significantly elevated anti-rLI0794 IgA antibodies in the ileum (p<0.01) relative to Group 4 (FIG. 14J). These data suggest that the intramuscularly administered subunit vaccines formulated with EMULSIGEN triggered antigen-specific mucosal humoral response in the intestine.

PBMCs were collected from the pigs prior to bacterial challenge. The cells were restimulated ex vivo with rLI0625, rLI0710, rLI0794, rLI0726, rLI0169 and rLI0786 and IFNγ production was quantified as a measure of T cell immune response. The results indicated that the cells were responsive to ConA indicating that the assay was working and the cells were alive but they did not produce IFNγ in any of the groups of pigs. These data suggest that at least when adjuvanted with EMULSIGEN, the subunit vaccines delivered via the intramuscular route failed to trigger a cellular immune response.

### Challenge Study:

For all the piglets in the trial above, they were fasted overnight then challenged orally (gavaged) on day 27 with approximately 1.9×10⁸ pathogenic L. intracellularis in 40 ml. Clinical scores showed no change in rectal temperature. Weights over the course of the immunization and challenge trial were not significantly different across any groups (FIG. 14K). Fecal samples were collected for 18 days and showed that Group 4 had no evidence of L. intracellularis (as expected for this no-challenge control group). When quantifying the number of animals that had very higher concentration of L. intracellularis DNA in feces (red boxes, Table 4) at any time point, there were 5/7 from Group 3 (Challenged, unvaccinated), 2/8 from Group 2 (Vaccinated with rLI0794, rLI0726 and rLI0786 with EMULSIGEN) and 0/7 from Group 1 (Vaccinated with rLI0710, rLI0625 and rLI0169 with EMULSIGEN). In fact, low level L. intracellularis DNA was found in 3/7 pigs from Group 1, 1/8 pigs from Group 2 and 0/7 pigs from Group 4.

Vaccines comprised of 3 recombinant proteins formulated with EMULSIGEN and administered by the intramuscular route triggered systemic and mucosal humoral immunity but they did not show a cell-mediated immune response, at least when formulated with EMULSIGEN. The vaccinated pigs in Group 1 showed protection against infectious challenge.

Trial 3: Mucosal Vaccination with Recombinant LI0710 Protein Formulated with VIDO Triple Adjuvant Triggered Robust Antigen-Specific and Cell-Mediated Immunity.

We investigated whether immunizing pigs by a mucosal route and replacing EMULSIGEN with VIDO Triple Adjuvant could impact the cell-mediated immune response to rLI0710 subunit protein. To test this, gilts were immunized at breeding with 1 mL total volume of 400 μg rLI0710 formulated with 1200 μg poly IC, 2400 μg host defense peptide 1002, and 1200 μg polyphosphazene VIDO Triple Adjuvant (n=8). The vaccine was added to 80 mL commercial extended semen (PIC Genetics) which had been subjected to repeated −80° C. to 37° C. temperature changes to kill but not rupture the semen. For the second and third doses, gilts were bred with killed semen and then live semen and VIDO Triple Adjuvant. By including killed semen, the gilts return to estrus after 21 day intervals. At these latter estrus cycles, vaccinated pigs were also immunized by the intramuscular route with 400 μg rLI0710 formulated with 400 μg poly IC, 800 μg host defense peptide 1002, and 400 μg polyphosphazene. Control animals were bred without anything added to the semen and without intramuscular administration of VIDO Triple Adjuvant (Mock, n=10).

Approximately 38 days after the last immunization, PBMCs were obtained and the cells were tested for ex vivo response to antigen. Results show that PBMCs from intrauterine-immunized and mock-immunized pigs produced IFNγ in response to Conconavalin A (ConA; closed and open circles), a mitogen included as a positive control (FIG. 15B). When no antigen was introduced to the isolated PBMCs, little IFNγ was produced indicating that the animals had low level activity of the isolated T cells (Media, closed and open squares), as expected. However, in the intrauterine-immunized gilts, we observed significantly higher levels of IFNγ production (p<0.001) when cells were re-exposed to rLI0710, relative to the mock-immunized gilts. These data suggest that the route and/or adjuvant combination triggered a cell-mediated immune response to rLI0710 in adult pigs.

Vaccines comprised of rLI0710 formulated with VIDO Triple Adjuvant combination and administered into the uterus followed up by two doses of intramuscular immunization triggered antigen-specific cell-mediated immunity.

All citations are hereby incorporated by reference. In the event of conflicting information with statements between any reference to or incorporated herein, and the present disclosure, the present disclosure will act as the guiding authority.

### REFERENCES

1. Pornwiroon, et al. (Proteomic analysis of Rickettsia parkeri strain portsmouth.) Infect Immun. 2009 December; 77(12):5262-71. doi: 10.1128/IAI.00911-09. Epub 2009 Sep. 21.
2. Pornwiroon, et al. (Immunoproteomic profiling of Rickettsia parkeri and Rickettsia amblyommii.) Ticks Tick Borne Dis. 2015 September; 6(6):829-35. doi: 10.1016/j.ttbdis.2015.07.012. Epub 2015 Jul. 22.
3. Simo G, Herder S, Cuny G, Hoheisel J. Identification of subspecies specific genes differentially expressed in procyclic forms of Trypanosoma brucei subspecies. Infection, Genetics and Evolution. 2010; 10:229-37.
4. F. Liu, X. Tang, X. Sheng, J. Xing, W. Zhan, DNA vaccine encoding molecular chaperone GroEL of Edwardsiella tarda confers protective efficacy against edwardsiellosis, Mol. Immunol., 79 (2016), pp. 55-65, 10.1016/j.molimm.2016.09.024
5. Natarajaseenivasan K1, Shanmughapriya S, Velineni S, Artiushin S C, Timoney J F. Cloning, expression, and homology modeling of GroEL protein from Leptospira interrogans serovar autumnalis strain N2. Genomics Proteomics Bioinformatics. 2011 October; 9(4-5):151-7. doi: 10.1016/S1672-0229(11)60018-1.
6. Luu L D W1, Octavia S1, Aitken C1, Zhong L2, Raftery M J2, Sintchenko V3, Lan R4. Surfaceome analysis of Australian epidemic Bordetella pertussis reveals potential vaccine antigens. Vaccine. 2020 Jan. 16; 38(3):539-548. doi: 10.1016/j.vaccine.2019.10.062. Epub 2019 Nov. 6.
7. Moreno-Cinos C et al, ClpP Protease, a Promising Antimicrobial Target. Int J Mol Sci. 2019 May 7; 20(9). pii: E2232. doi: 10.3390/ijms20092232.
8. Wu et al, Immunization with a combination of three pneumococcal proteins confers additive and broad protection against Streptococcus pneumoniae Infections in Mice. Infect Immun. 2010 March; 78(3):1276-83. doi: 10.1128/IAI.00473-09. Epub 2009 Dec. 28.
9. Cao J1, CD4(+) T lymphocytes mediated protection against invasive pneumococcal infection induced by mucosal immunization with ClpP and CbpA. Vaccine. 2009 May 11; 27(21):2838-44. doi: 10.1016/j.vaccine.2009.02.093. Epub 2009 Mar. 10.

1. An immunogenic, subunit composition comprising at least one isolated, immunogenic Lawsonia intracellularis protein selected from an LI0710, an LI0649, an LI0169, an LI1153, an LI0786, an LI1171, an LI0608, an LI0726, an LI0823, an LI0625, an LI0794, an immunogenic fragment thereof, an immunogenic variant thereof, or the corresponding protein from another L. intracellularis strain or isolate, and a pharmaceutically acceptable excipient.

2. The immunogenic composition of claim 1, wherein the L. intracellularis protein(s) is selected from one or more proteins comprising the amino acid sequence of SEQ ID NOs: 2, 5, 8, 11, 14, 17, 20, 23, 26, 29 and 32, an immunogenic fragment thereof, or an immunogenic fragment or variant thereof.

3. The immunogenic composition of claim 2, wherein the L. intracellularis immunogenic protein comprises the amino acid sequence of SEQ ID NOs: 2, 5, 8, 11, 14, 17, 20, 23, 26, 29 or 32, with a deletion of all or part of a transmembrane binding domain or a native signal sequence, if present.

4. The immunogenic composition of claim 1, wherein the L. intracellularis immunogenic protein comprises an amino acid sequence with at least 90% sequence identity to the amino acid sequence of SEQ ID NOs: 2, 5, 8, 11, 14, 17, 20, 23, 26, 29 or 32.

5. The immunogenic composition of claim 4, wherein the L. intracellularis immunogenic protein comprises the amino acid sequence of amino acids 2-293 of SEQ ID NO: 2.

6. The immunogenic composition of claim 4, wherein the L. intracellularis immunogenic protein comprises the amino acid sequence of amino acids 31-851 of SEQ ID NO: 5.

7. The immunogenic composition of claim 4, wherein the L. intracellularis immunogenic protein comprises the amino acid sequence of amino acids 43-552 of SEQ ID NO: 8.

8. The immunogenic composition of claim 4, wherein the L. intracellularis immunogenic protein comprises the amino acid sequence of amino acids 5-398 of SEQ ID NO: 11.

9. The immunogenic composition of claim 4, wherein the L. intracellularis immunogenic protein comprises the amino acid sequence of amino acids 1-383 of SEQ ID NO: 14.

10. The immunogenic composition of claim 4, wherein the L. intracellularis immunogenic protein comprises the amino acid sequence of amino acids 1-562 of SEQ ID NO: 17.

11. The immunogenic composition of claim 4, wherein the L. intracellularis immunogenic protein comprises the amino acid sequence of amino acids 1-485 of SEQ ID NO: 20.

12. The immunogenic composition of claim 4, wherein the L. intracellularis immunogenic protein comprises the amino acid sequence of amino acids 1-404 of SEQ ID NO: 23.

13. The immunogenic composition of claim 4, wherein the L. intracellularis immunogenic protein comprises the amino acid sequence of amino acids 1-363 of SEQ ID NO: 26.

14. The immunogenic composition of claim 4, wherein the L. intracellularis immunogenic protein comprises the amino acid sequence of amino acids 1-548 of SEQ ID NO: 29.

15. The immunogenic composition of claim 4, wherein the L. intracellularis immunogenic protein comprises the amino acid sequence of amino acids 1-209 of SEQ ID NO: 32.

16. The immunogenic composition of claim 1, comprising two or more isolated immunogenic L. intracellularis proteins.

17. The immunogenic composition of claim 1, wherein two or more immunogenic L. intracellularis proteins are present and the two or more proteins are provided as a fusion protein.

18. The immunogenic composition of claim 1, further comprising an immunological adjuvant.

19. The immunogenic composition of claim 18, wherein the immunological adjuvant comprises an oil-in-water emulsion.

20. The immunogenic composition of claim 18, wherein the immunological adjuvant comprises (a) a polyphosphazene; (b) a poly(I:C) or a CpG oligonucleotide; and (c) a host defense peptide.

21. The immunogenic composition of claim 20, wherein the immunological adjuvant is in the form of a microparticle.

22. The immunogenic composition of claim 21, wherein the polyphosphazene is PCEP and the host defense peptide is peptide 1002.

23. A recombinant vector comprising:
(a) a DNA molecule encoding an immunogenic L. intracellularis protein selected from an LI0710, an LI0649, an LI0169, an LI1153, an LI0786, an LI1171, an LI0608, an LI0726, an L10823, an L10625, an L10794, an immunogenic fragment thereof, an immunogenic variant thereof, or the corresponding protein from another L. intracellularis strain or isolate; and
(b) control elements that are operably linked to said molecule whereby a coding sequence in said molecule can be transcribed and translated in a host cell.

24. A host cell transformed with the recombinant vector of claim 23.

25. The host cell of claim 24, wherein the host cell is an E. coli cell.

26. A method of producing a L. intracellularis protein comprising:
(a) providing a population of host cells according to claim 25; and
(b) culturing said population of cells under conditions whereby the protein encoded by the DNA molecule present in said recombinant vector is expressed.

27. A method of treating or preventing a L. intracellularis infection in a vertebrate subject comprising administering a therapeutic amount of the composition of claim 1 to the subject.

28. The method of claim 27, wherein the subject is a porcine or equine subject.

29. The method of claim 28, wherein the L. intracellularis infection comprises a proliferative enteropathy.
